(19) United States
(12) Patent Application Publication
(10) Pub. No.: US 2011/0318780 A1
(43) Pub. Date: Dec. 29, 2011

(54) ENZYMATIC MODIFICATION OF GLYCOPEPTIDES

(75) Inventor: Shawn DeFrees, North Wales, PA (US)

(73) Assignee: BIOGENERIX AG, Mannheim (DE)

(21) Appl. No.: 13/163,473

(22) Filed: Jun. 17, 2011

Related U.S. Application Data
(62) Division of application No. 11/658,218, filed on Jun. 20, 2008, now abandoned.
(60) Provisional application No. 60/611,790, filed on Sep. 20, 2004.
(60) Provisional application No. 60/590,649, filed on Jul. 23, 2004.

(51) Int. Cl. C12P 21/00; C12P 21/06

(52) U.S. Cl. 435/68.1

(57) ABSTRACT

The present invention provides glycoconjugates that are formed through the enzymatically-mediated coupling of a glycosyl moiety, e.g., on a peptide or lipid, and a modifying group that includes an acyl group. The conjugates include the modifying group tethered to the glycosyl moiety through a linking moiety that includes an acyl residue. Also provided are methods for preparing the conjugates of the invention

### CROSS-REFERENCES TO RELATED APPLICATIONS

The present application is a U.S. national phase application of PCT Patent Application No. PCT/US2005/026377, and claims priority to U.S. Provisional Patent Application No. 60/611,790, filed Sep. 20, 2004 and U.S. Provisional Patent Application No. 60/590,649, filed Jul. 23, 2004; each of which are incorporated by reference in their entirety for all purposes.

### BACKGROUND OF THE INVENTION

1. Field of the Invention

The present invention relates to corrugates formed between a glycosyl-containing species (e.g., glycopeptide, glycolipid) and a modifying group. The glycosyl-containing species and modifying group are linked through an enzymatically formed acyl-containing bond (e.g., amide, ester). The glycosyl-containing species are typically therapeutic agents.

2. Background

The administration of glycosylated and non-glycosylated therapeutic agents for engendering a particular physiological response is well known in the medicinal arts. For example, both purified and recombinant hGH are used for treating conditions and diseases due to hGH deficiency, e.g., dwarfism in children, interferon has known antiviral activity and granulocyte colony stimulating factor stimulates the production of white blood cells.

A principal factor that has limited the use of therapeutic peptides is the difficulty inherent in engineering an expression system to express a peptide having the glycosylation pattern of the wild-type peptide. Improperly or incompletely glycosylated peptides can be immunogenic; in a patient, an immunogenic response to an administered peptide can neutralize the peptide and/or lead to the development of an allergic response in the patient. Other deficiencies of recombinantly produced glycopeptides include suboptimal potency and rapid clearance rates. The problems inherent in peptide therapeutics are recognized in the art, and various methods of eliminating the problems have been investigated.

Post-expression in vitro modification of peptides is an attractive strategy to remedy the deficiencies of methods that rely on controlling glycosylation by engineering expression systems; including both modification of glycan structures or introduction of glycans at novel sites. A comprehensive toolbox of recombinant eukaryotic glycosyltransferases is becoming available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; and WO/9831826; US2003180835; and WO 03/031464.

Enzyme-based syntheses have the advantages of regioselectivity and stereoselectivity. Moreover, enzymatic syntheses are performed using unprotected substrates. Two principal classes of enzymes are used in the synthesis of carbohydrates, glycosyltransferases (e.g., sialyltransferases, oligosaccharyltransferases, N-acetylglucosaminyltransferases), and glycosidases. The glycosidases are further classified as exoglycosidases (e.g., β-mannosidase, β-glucosidase), and endoglycosidases (e.g., Endo-A, Endo-M). Each of these classes of enzymes has been successfully used synthetically to prepare carbohydrates. For a general review, see, Crout et al., Curr. Opin. Chem. Biol. 2: 98-111 (1998).

Glycosyltransferases modify the oligosaccharide structures on glycopeptides. Glycosyltransferases are effective for producing specific products with good stereochemical and regiochemical control. Glycosyltransferases have been used to prepare oligosaccharides and to modify terminal N- and O-linked carbohydrate structures, particularly on glycopeptides produced in mammalian cells. For example, the terminal oligosaccharides of glycopeptides have been completely sialylated and/or fucosylated to provide more consistent sugar structures, which improves glycopeptide pharmacodynamics and a variety of other biological properties. For example, β-1,4-galactosyltransferase was used to synthesize lactosamine, an illustration of the utility of glycosyltransferases in the synthesis of carbohydrates (see, e.g., Wong et al., J. Org. Chem. 47: 5416-5418 (1982)). Moreover, numerous synthetic procedures have made use of α-sialyltransferases to transfer sialic acid from cytidine-5′-monophospho-N-acetylneuraminic acid to the 3-OH or 6-OH of galactose (see, e.g., Kevin et al., Chem. Eur. J. 2: 1359-1362 (1996)). Fucosyltransferases are used in synthetic pathways to transfer a fucose unit from guanosine-5′-diphosphofucose to a specific hydroxyl of a saccharide acceptor. For example, Ichikawa prepared sialyl Lewis-X by a method that involves the fucosylation of sialylated lactosamine with a cloned fucosyltransferase (Ichikawa et al., J. Am. Chem. Soc. 114: 9283-9298 (1992)). For a discussion of recent advances in glycoconjugate synthesis for therapeutic use see, Koeller et al., Nature Biotechnology 18: 835-841 (2000). See also, U.S. Pat. Nos. 5,876,980; 6,030,815; 5,728,554; 5,922,577; and WO/9831826.

Glycosidases can also be used to prepare saccharides. Glycosidases normally catalyze the hydrolysis of a glycosidic bond. Under appropriate conditions, however, they can be used to form this linkage. Most glycosidases used for carbohydrate synthesis are exoglycosidases; the glycosyl transfer occurs at the non-reducing terminus of the substrate. The glycosidase takes up a glycosyl donor in a glycosyl-enzyme intermediate that is either intercepted by water to give the hydrolysis product, or by an acceptor, to give a new glycoside or oligosaccharide. An exemplary pathway using an exoglycosidase is the synthesis of the core trisaccharide of all N-linked glycopeptides, including the difficult β-mannoside linkage, which was formed by the action of β-mannosidase (Singh et al., Chem. Commun. 993-994 (1996)).

In another exemplary application of the use of a glycosidase to form a glycosidic linkage, a mutant glycosidase was prepared in which the normal nucleophilic amino acid within the active site is changed to a non-nucleophilic amino acid. The mutant enzymes do not hydrolyze glycosidic linkages, but can still form them. The mutant glycosidases are used to prepare oligosaccharides using an α-glycosyl fluoride donor and a glycoside acceptor molecule (Withers et al., U.S. Pat. No. 5,716,812). Although the mutant glycosidases are useful for forming free oligosaccharides, it has yet to be demonstrated that such enzymes are capable of appending glycosyl donors onto glycosylated or non-glycosylated peptides, nor have these enzymes been used with unactivated glycosyl donors.

Although their use is less common than that of the exoglycosidases, endoglycosidases are also utilized to prepare carbohydrates. Methods based on the use of endoglycosidases have the advantage that an oligosaccharide, rather than a monosaccharide, is transferred. Oligosaccharide fragments have been added to substrates using endo-β-N-acetylglucosamines such as endo-F, endo-M (Wang et al., Tetrahedron Lett. 37: 1975-1978); and Haneda et al., Carbohydr. Res. 292: 61-70 (1996)).

In addition to their use in preparing carbohydrates, the enzymes discussed above are applied to the synthesis of glycopeptides. The synthesis of a homogenous glycoform of ribonuclease B has been published (Witte K. et al., J. Am. Chem. Soc. 119: 2114-2118 (1997)). The high mannose core of ribonuclease B was cleaved by treating the glycopeptide with endoglycosidase H. The cleavage occurred specifically between the two core GlcNAc residues. The tetrasaccharide sialyl Lewis X was then enzymatically rebuilt on the remaining GlcNAc anchor site on the now homogenous protein by the sequential use of β-1,4-galactosyltransferase, α-2,3-sialyltransferase and α-1,3-fucosyltransferase V. Each enzymatically catalyzed step proceeded in excellent yield.

Methods combining both chemical and enzymatic synthetic elements are also known. For example, Yamamoto and coworkers (Carbohydr. Res. 305: 415-422 (1998)) reported the chemoenzymatic synthesis of the glycopeptide, glycosylated Peptide T, using an endoglycosidase. The N-acetylglucosaminyl peptide was synthesized by purely chemical means. The peptide was subsequently enzymatically elaborated with the oligosaccharide of human transferrin glycopeptide. The saccharide portion was added to the peptide by treating it with an endo-β-N-acetylglucosaminidase. The resulting glycosylated peptide was highly stable and resistant to proteolysis when compared to the peptide T and N-acetylglucosaminyl peptide T.

The use of glycosyltransferases to modify peptide structure with reporter groups has been explored. For example, Brossmer et al. (U.S. Pat. No. 5,405,753) discloses the formation of a fluorescent-labeled cytidine monophosphate (“CMP”) derivative of sialic acid and the use of the fluorescent glycoside in an assay for sialyl transferase activity and for the fluorescent labeling of cell surfaces, glycoproteins and gangliosides. Gross et al. (Analyt. Biochem. 186: 127 (1990)) describe a similar assay. Bean et al. (U.S. Pat. No. 5,432,059) discloses an assay for glycosylation deficiency disorders utilizing reglycosylation of a deficiently glycosylated protein. The deficient protein is reglycosylated with a fluorescent-labeled CMP glycoside. Each of the fluorescent sialic acid derivatives is substituted with the fluorescent moiety at either the 9-position or at the amine that is normally acetylated in sialic acid. The methods using the fluorescent sialic acid derivatives are assays for the presence of glycosyltransferases or for non-glycosylated or improperly glycosylated glycoproteins. The assays are conducted on small amounts of enzyme or glycoprotein in a sample of biological origin. The enzymatic derivatization of a glycosylated or non-glycosylated peptide on a preparative or industrial scale using a modified sialic acid has not been disclosed or suggested.

Considerable effort has also been directed towards the modification of cell surfaces by altering glycosyl residues presented by those surfaces. For example, Fukuda and coworkers have developed a method for attaching glycosides of defined structure onto cell surfaces. The method exploits the relaxed substrate specificity of a fucosyltransferase that can transfer fucose and fucose analogs bearing diverse glycosyl substrates (Tsuboi et al., J. Biol. Chem. 271: 27213 (1996)).

The methods of modifying cell surfaces have not been applied in the absence of a cell to modify a glycosylated or non-glycosylated peptide. Moreover, the methods of cell surface modification are not utilized for the enzymatic incorporation preformed modified glycosyl donor moiety into a peptide. Moreover, none of the cell surface modification methods are practical for producing glycosyl-modified peptides on an industrial scale.

Enzymatic methods have also been used to activate glycosyl residues on a glycopeptide towards subsequent chemical elaboration. The glycosyl residues are typically activated using galactose oxidase, which converts a terminal galactose residue to the corresponding aldehyde. The aldehyde is subsequently coupled to an amine-containing modifying group. For example, Casares et al. (Nature Biotech. 19: 142 (2001)) have attached doxorubicin to the oxidized galactose residues of a recombinant MHCII-peptide chimera.

In addition to manipulating the structure of glycosyl groups on polypeptides, interest has developed in preparing glycopeptides that are modified with one or more non-saccharide modifying group, such as water soluble polymers. Poly(ethyleneglycol) (“PEG”) is an exemplary polymer that has been conjugated to polypeptides. The use of PEG to derivatize peptide therapeutics has been demonstrated to reduce the immunogenicity of the peptides. For example, U.S. Pat. No. 4,179,337 (Davis et al.) discloses non-immunogenic polypeptides, such as enzymes and peptide hormones coupled to polyethylene glycol (PEG) or polypropylene glycol. Between 10 and 100 moles of polymer are used per mole polypeptide. Although the in vivo clearance time of the conjugate is prolonged relative to that of the polypeptide, only about 15% of the physiological activity is maintained. Thus, the prolonged circulation half-life is counterbalanced by the dramatic reduction in peptide potency.

The loss of peptide activity is directly attributable to the non-selective nature of the chemistries utilized to conjugate the water-soluble polymer. The principal mode of attachment of PEG, and its derivatives, to peptides is a non-specific bonding through a peptide amino acid residue. For example, U.S. Pat. No. 4,088,538 discloses an enzymatically active polymer-enzyme conjugate of an enzyme covalently bound to PEG. Similarly, U.S. Pat. No. 4,496,689 discloses a covalently attached complex of α-1 proteinase inhibitor with a polymer such as PEG or methoxypoly(ethyleneglycol) (“(m-) PEG”). Abuchowski et al. (J. Biol. Chem. 252: 3578 (1977)) discloses the covalent attachment of (m-) PEG to an amine group of bovine serum albumin. U.S. Pat. No. 4,414,147 discloses a method of rendering interferon less hydrophobic by conjugating it to an anhydride of a dicarboxylic acid, such as poly(ethylene succinic anhydride). PCT WO 87/00056 discloses conjugation of PEG and poly(oxyethylated) polyols to such proteins as interferon-β, interleukin-2 and immunotoxins. EP 154,316 discloses and claims chemically modified lymphokines, such as IL-2 containing PEG bonded directly to at least one primary amino group of the lymphokine. U.S. Pat. No. 4,055,635 discloses pharmaceutical compositions of a water-soluble complex of a proteolytic enzyme linked covalently to a polymeric substance such as a polysaccharide.

Another mode of attaching PEG to peptides is through the non-specific oxidation of glycosyl residues on a glycopeptide. The oxidized sugar is utilized as a locus for attaching a PEG moiety to the peptide. For example M'Timkulu (WO 94/05332) discloses the use of an amino-PEG to add PEG to a glycoprotein. The glycosyl moieties are randomly oxidized to the corresponding aldehydes, which are subsequently coupled to the amino-PEG.

In each of the methods described above, poly(ethyleneglycol) is added in a random, non-specific manner to reactive residues on a peptide backbone. For the production of therapeutic peptides, it is clearly desirable to utilize a derivitization strategy that results in the formation of a specifically labeled, readily characterizable, essentially homogeneous product. A promising route to preparing specifically labeled peptides is through the use of enzymes, such as glycosyltransferases to append a modified sugar moiety onto a peptide.

Glycosyl residues have also been modified to bear ketone groups. For example, Mahal and co-workers (Science 276: 1125 (1997)) have prepared N-levulinoyl mannosamine (“ManLev”), which has a ketone functionality at the position normally occupied by the acetyl group in the natural substrate. Cells were treated with the ManLev, thereby incorporating a ketone group onto the cell surface. See, also Saxon et al., Science 287: 2007 (2000); Hang et al., J. Am. Chem. Soc. 123: 1242 (2001); Yarema et al., J. Biol. Chem. 273: 31168 (1998); and Charter et al., Glycobiology 10: 1049 (2000).

In addition to an industrially relevant method that utilizes the enzymatic conjugation to specifically conjugate a modified sugar to a peptide or glycopeptide, a method for controlling and manipulating the position of glycosylation on a glycopeptide would be highly desirable.

Carbohydrates are attached to glycopeptides in several ways of which N-linked to asparagine and mucin-type O-linked to serine and threonine are the most relevant for recombinant glycoprotein therapeutics. A determining factor for initiation of glycosylation of a protein is the primary sequence context, although clearly other factors including protein region and conformation play roles. N-linked glycosylation occurs at the consensus sequence NXS/T, where X can be any amino acid but proline.

O-linked glycosylation is initiated by a family of about 20 homologous enzymes termed UDP-GalNAc: polypeptide N-acetylgalactosaminyltransferases (GalNAc-transferases). O-linked glycosylation does not appear to be ruled by one simple consensus sequence, although studies of the GalNAc-transferase enzymes that initiate O-linked glycosylation clearly supports the notion that their acceptor specificities are driven by primary sequence contexts. Each of these enzymes transfer a single monosaccharide GalNAc to serine and threonine residues, but they transfer to different peptide sequences although they show a large degree of overlap in functions. It is envisioned that the substrate specificity of each GalNAc-transferase is ruled primarily by a linear short acceptor consensus sequence.

Recently, a method of producing an ester linked carbohydrate-peptide conjugate was described by Davis (WO 03/014371, published Feb. 20, 2003). In this publication, a vinyl ester amino acid group was reacted with a carbohydrate acyl acceptor in the presence of an enzyme such as a protease (such as a serine protease), lipase, esterase or acylase. At this time, however, no other substrates, e.g., glycopeptides, glycolipids, are known to conjugate with carbohydrate acyl acceptors under these conditions.

The present invention answers the need for modified therapeutic species in which a modified glycosyl moiety is conjugated onto N- or O-linked glycosylation sites of the peptides and other bioactive species, e.g., glycolipids, sphingosines, ceramides, etc. The invention provides a route to new therapeutic conjugates and addresses the need for more stable and therapeutically effective therapeutic species. Moreover, despite the efforts directed toward the enzymatic elaboration of saccharide structures, there remains still a need for alternative industrially practical methods for the modification of therapeutic agents, e.g., peptides, glycopeptides and lipids with modifying groups such as water-soluble polymers, therapeutic moieties, biomolecules and the like. Of particular interest are methods in which the modified peptide has improved properties, which enhance its use as a therapeutic or diagnostic agent. The present invention fulfills these and other needs.

### BRIEF SUMMARY OF THE INVENTION

Glycotherapeutics (e.g., glycopeptides, and glycolipids) present a challenging target for recombinant production of therapeutics. For example, carbohydrates are often indispensable for the function and favorable pharmacokinetic properties of glycopeptide therapeutics; however, many of the most robust expressions systems produce glycopeptides with non-human glycosylation patterns. Incorrect glycosylation can produce a peptide that is inactive, aggregated, antigenic and/or has unfavorable pharmacokinetics. Accordingly, considerable efforts are expended to develop recombinant expression cell systems capable of producing glycoproteins with biologically appropriate carbohydrate structures. This approach is hampered by numerous shortcomings, including cost, and heterogeneity and limitations in glycan structures.

Post-expression in vitro glyco-modification of glycotherapeutics, e.g., glycopeptides, is an attractive strategy to remedy the deficiencies of methods that rely on controlling glycosylation by engineering expression systems; including both modification of glycan structures or introduction of glycans at novel sites. A comprehensive toolbox of recombinant eukaryotic glycosyltransferases is becoming available, making in vitro enzymatic synthesis of mammalian glycoconjugates with custom designed glycosylation patterns and glycosyl structures possible. See, for example, U.S. Pat. Nos. 5,876,980; 6,030,815; 5,728,554; 5,922,577; and WO/9831826; US2003180835; and WO 03/031464.

In vitro glycosylation offers a number of advantages compared to recombinant expression of glycoproteins of which custom design and higher degree of homogeneity of the glycosyl moiety are examples. Moreover, combining bacterial expression of glycotherapeutics with in vitro modification (or placement) of the glycosyl residue offers numerous advantages over traditional recombinant expression technology including reduced potential exposure to adventitious agents, increased homogeneity of product, and cost reduction.

Ideally, therapeutic conjugates of glycosyl-containing species, such as glycopeptides and glycolipids, are obtained using methods that provide the conjugates in a reproducible and predictable manner. Moreover, in forming the conjugates it is generally preferred that the site of conjugation between the glycosyl-containing species and the modifying group is selected such that its modification does not adversely affect advantageous properties of glycosyl-containing species, e.g. activity, specificity, low antigenicity, low toxicity, etc.

The present invention provides an enzymatically-mediated method of forming conjugates between a glycosyl residue, amino acid residue (e.g., NH, OH, SH) or aglycone acid of a selected substrate (e.g., glycopeptide, glycolipid, etc.) and a modifying group, such as a water-soluble or water-insoluble polymer, a therapeutic moiety or a diagnostic agent. The invention exploits the recognition that certain classes of enzymes, which are typically degradative, can be made to run in a synthetic, rather than a degradative mode. Exemplary enzymes are those that are involved in the cleavage of bonds that include an acyl-containing component, such as an ester or an amide. Thus, enzymes of use in the present invention include, but are not limited to, proteases, lipases, acylases, and esterases. The invention can also be practiced with enzymes that are involved in the transfer of an acyl-containing moiety onto a substrate, e.g., acyltransferases, and amino acid t-RNA transferase.

In an exemplary aspect, the invention provides a lipid or peptide conjugate that includes a glycosyl residue having the formula:

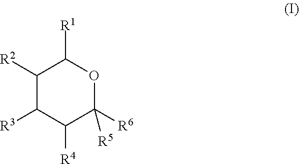

in which the symbols R¹, R², R³, R⁴, and R⁵ independently represent H, OR^{7a}, N(R^{7a})₂, SR^{7a}, JC(O)R⁷, substituted or unsubstituted alkyl, substituted or unsubstituted heteroalkyl, substituted or unsubstituted aryl, substituted or unsubstituted heteroaryl or substituted or unsubstituted heterocycloalkyl. The symbol J represents a bond, O, S or NH. The symbol R⁷ represents H, OR⁸, NR⁸R⁹, substituted or unsubstituted alkyl, substituted or unsubstituted heteroalkyl, substituted or unsubstituted aryl, substituted or unsubstituted heteroaryl, or substituted or unsubstituted heterocycloalkyl. Each R^{7a} is independently selected and represents H, substituted or unsubstituted alkyl, substituted or unsubstituted heteroalkyl, substituted or unsubstituted aryl, substituted or unsubstituted heteroaryl, or substituted or unsubstituted heterocycloalkyl.

R⁸ and R⁹ independently represent H, substituted or unsubstituted alkyl, substituted or unsubstituted heteroalkyl, substituted or unsubstituted aryl, substituted or unsubstituted heteroaryl, or substituted or unsubstituted heterocycloalkyl.

In the conjugates of the invention, at least one of R¹, R², R³, R⁴, and R⁵ comprises a modifying group (e.g., polymer, therapeutic moiety, etc.) as discussed herein. The modifying group is linked to the glycosyl residue through a moiety that includes a carbonyl group, e.g., an acyl group (e.g., ROC(O)R′). In an exemplary embodiment, one of R⁸ or R⁹ is a water-soluble polymer moiety (e.g., m-PEG, branched m-PEG).

The symbol R⁶ represents an amino acid residue of the peptide, a carbohydrate linker moiety covalently bound to an amino acid residue of the peptide, and combinations thereof.

Alternatively, when the conjugate includes a lipid, R⁶ represents an aglycone, a carbohydrate linker moiety covalently bound to an aglycone, and combinations thereof.

When R⁶ is a carbohydrate linker moiety, exemplary moieties bound to the glycosyl core shown in Formula I include Gal, GalNAc, Man, GlcNAc, Fuc, Sia, and Glu.

The invention also includes methods of preparing an acyl-modified glycosyl conjugate utilizing a mutant enzyme and mutant enzymes of use in the method. The mutant enzymes include a residue in the active site that is not found in the corresponding wild-type peptide. The residue acts to diminish or eliminate the hydrolytic activity of the enzyme. Art-recognized methods for preparing mutant peptides and screening them for a desired activity are of use in the present invention.

The invention also provides methods of improving pharmacological parameters of glycotherapeutics. For example, the invention provides a means for altering the pharmacokinetics, pharmacodynamics and bioavailability of glycosyl-containing therapeutics, e.g., cytokines, antibodies, growth hormones, enzymes, and glycolipids. In particular, the invention provides a method for lengthening the in vivo half-life of a glycotherapeutic by conjugating a water-soluble polymer to the therapeutic moiety through an acylated glycosyl linking group, e.g., an intact glycosyl linking group, or an acylated amino acid. In an exemplary embodiment, covalent attachment of polymers, such as polyethylene glycol (PEG), e.g., m-PEG, to a therapeutic moiety affords conjugates having in vivo residence times, and pharmacokinetic and pharmacodynamic properties that are enhanced relative to the unconjugated therapeutic.

As discussed in the preceding section, art-recognized methods of covalent PEGylation rely on non-selective chemical conjugation through reactive groups, typically amines, on amino acids or carbohydrates. A major shortcoming of chemical conjugation of PEG to proteins or glycoproteins is lack of selectivity, which often results in attachment of PEG at sites implicated in protein or glycoprotein bioactivity. Several strategies have been developed to address non-enzymatic site selective conjugation chemistries, however, one universal method suitable for a variety of recombinant proteins has yet to be developed.

In contrast to art-recognized chemical conjugation methods, the present invention provides a novel, enzymatically-mediated strategy for selective conjugation, e.g., PEGylation, directed to one or more specific location on a glycosyl residue of a glycopeptide or glycolipid. In an exemplary embodiment of the invention, site directed attachment of PEG is provided by in vitro enzymatic acylation of specific residues on a glycosyl moiety by an activated PEG compound.

Additional aspects, advantages and objects of the present invention will be apparent from the detailed description that follows.

### DESCRIPTION OF THE DRAWINGS

FIG. 1 is a table presenting exemplary sialyltransferases of use in the present invention.

### DETAILED DESCRIPTION OF THE INVENTION

### Abbreviations

Branched or un-branched PEG, poly(ethyleneglycol), including m-PEG, methoxy-poly(ethylene glycol); branched or unbranched PPG, poly(propyleneglycol), including m-PPG, methoxy-poly(propylene glycol); Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Sia, sialic acid; and NeuAc, N-acetylneuraminyl.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references, which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—C₁₋₁₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroakyl substituents,” respectively, and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, the aryl substituents and heteroaryl substituents are generally referred to as “aryl substituents” and “heteroaryl substituents,” respectively and are varied and selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S) and silicon (Si).

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “mutating” or “mutation,” as used in the context of altering the structure or enzymatic activity of a wild-type enzyme, refers to the deletion, insertion, or substitution of any nucleotide or amino acid residue, by chemical, enzymatic, or any other means, in a polynucleotide sequence encoding a that enzyme or the amino acid sequence of a wild-type enzyme, respectively, such that the amino acid sequence of the resulting enzyme is altered at one or more amino acid residues. The site for such an activity-altering mutation may be located anywhere in the enzyme, but is preferably within the active site of the enzyme.

“Peptide” refers to a polymer in which the monomers are amino acids and are joined together through amide bonds, alternatively referred to as a polypeptide. Additionally, unnatural amino acids, for example, β-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are peptides that are incompletely glycosylated by a system that expresses the peptide. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “peptide conjugate,” refers to species of the invention in which a peptide is conjugated with an acyl-containing group that is attached to the peptide through a sugar residue.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

As used herein, the term “modified sugar,” refers to a naturally- or non-naturally-occurring carbohydrate of a glycosyl-containing compound to which an acyl-containing modifying group is added onto a glycosyl residue of a peptide in a process of the invention. The “modified sugar” is covalently functionalized with a “modifying group.” Useful modifying groups include, but are not limited to, water-soluble polymers, therapeutic moieties, diagnostic moieties, biomolecules and the like.

The term “water-soluble” refers to moieties that have a detectable degree of solubility in water. Methods to detect and/or quantify water solubility are well known in the art. Exemplary water-soluble polymers include peptides, saccharides, poly(ethers), poly(amines), poly(carboxylic acids) and the like. Peptides can have mixed sequences of be composed of a single amino acid, e.g., poly(lysine), poly(aspartic acid), and poly(glutamic acid). An exemplary polysaccharide is poly(sialic acid). An exemplary poly(ether) is poly(ethylene glycol), e.g., m-PEG. Poly(ethylene imine) is an exemplary polyamine, and poly(acrylic) acid is a representative poly(carboxylic acid).

The polymer backbone of the water-soluble polymer can be poly(ethylene glycol) (PEG). However, it should be understood that other related polymers are also suitable for use in the practice of this invention and that the use of the term PEG or poly(ethylene glycol) is intended to be inclusive and not exclusive in this respect. The term PEG includes poly(ethylene glycol) in any of its forms, including alkoxy PEG, alkyl PEG (e.g., mPEG), difunctional PEG, multiarmed PEG, forked PEG, branched PEG, pendent PEG (i.e. PEG or related polymers having one or more functional groups pendent to the polymer backbone), or PEG with degradable linkages therein.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, pentaerythritol and sorbitol. The central branch moiety can also be derived from several amino acids, such as lysine. The branched poly(ethylene glycol) can be represented in general form as R(-PEG-OH)ₘ in which R represents the core moiety, such as glycerol or pentaerythritol, and m represents the number of arms. Multi-armed PEG molecules, such as those described in U.S. Pat. Nos. 5,932,462; 5,643,575; European Patent Application 0473,084 A2; WO 96/41813 (and its priority documents), can also be used as the polymer backbone.

Many other polymers are also suitable for the invention. Polymer backbones that are non-peptidic and water-soluble, with from 2 to about 300 termini, are particularly useful in the invention. Examples of suitable polymers include, but are not limited to, other poly(alkylene glycols), such as poly(propylene glycol) (“PPG”), copolymers of ethylene glycol and propylene glycol and the like, poly(oxyethylated polyol), poly(olefinic alcohol), poly(vinylpyrrolidone), poly(hydroxypropylmethacrylamide), poly(α-hydroxy acid), poly(vinyl alcohol), polyphosphazene, polyoxazoline, poly(N-acryloylmorpholine), such as described in U.S. Pat. No. 5,629,384, which is incorporated by reference herein in its entirety, and copolymers, terpolymers, and mixtures thereof. Although the molecular weight of each chain of the polymer backbone can vary, it is typically in the range of from about 100 Da to about 100,000 Da, often from about 6,000 Da to about 80,000 Da.

The terms “large-scale” and “industrial-scale” are used interchangeably and refer to a reaction cycle that produces at least about 250 mg, preferably at least about 500 mg, and more preferably at least about 1 gram of glycoconjugate at the completion of a single reaction cycle.

The term, “glycosyl linking group,” as used herein refers to a glycosyl residue to which an acyl-containing modifying group (e.g., PEG moiety, therapeutic moiety, biomolecule) is covalently attached; the glycosyl linking group joins the modifying group to the remainder of the conjugate. In the methods of the invention, the “glycosyl linking group” is formed by the covalent modification, via an enzymatic acylation reaction of a glycosyl residue, thereby linking the agent to an amino acid and/or glycosyl residue on the peptide. The glycosyl linking group can be a saccharide-derived structure that is degraded or degraded and modified prior to the addition of the modifying group (e.g., oxidation→Schiff base formation→reduction). Alternatively, the glycosyl linking group may be intact. An “intact glycosyl linking group” refers to a linking group that is derived from a glycosyl moiety in which the saccharide monomer that links the modifying group and to the remainder of the conjugate is not degraded, e.g., oxidized, e.g., by sodium metaperiodate to create a locus of attachment for the modifying group. “Intact glycosyl linking groups” of the invention may be derived from a naturally occurring oligosaccharide by addition of glycosyl unit(s) or removal of one or more glycosyl unit from a parent saccharide structure.

As used herein, the terms “polymer” and “polymers” are used interchangeably with the terms “oligomer” and “oligomers.” The terms refer to species that have more than one structurally related subunit, e.g., oligosaccharide and polysaccharide.

The term “targeting moiety,” as used herein, refers to species that selectively localize in a particular tissue or region of the body. The localization is mediated by specific recognition of molecular determinants, molecular size of the targeting agent or conjugate, ionic interactions, hydrophobic interactions and the like. Other mechanisms of targeting an agent to a particular tissue or region are known to those of skill in the art. Exemplary targeting moieties include antibodies, antibody fragments, transferrin, HS-glycoprotein, coagulation factors, serum proteins, β-glycoprotein, G-CSF, GM-CSF, M-CSF, EPO and the like.

As used herein, “therapeutic moiety” means any agent useful for therapy including, but not limited to, antibiotics, anti-inflammatory agents, anti-tumor drugs, cytotoxins, and radioactive agents. “Therapeutic moiety” includes prodrugs of bioactive agents, constructs in which more than one therapeutic moiety is bound to a carrier, e.g., multivalent agents. Therapeutic moiety also includes proteins and constructs that include proteins. Exemplary proteins include, but are not limited to, Erythropoietin (EPO), Granulocyte Colony Stimulating Factor (GCSF), Granulocyte Macrophage Colony Stimulating Factor (GMCSF), Interferon (e.g., Interferon-α, -β, -γ), Interleukin (e.g., Interleukin II), serum proteins (e.g., Factors VII, VIIa, VIII, IX, and X), Human Chorionic Gonadotropin (HCG), Follicle Stimulating Hormone (FSH) and Lutenizing Hormone (LH) and antibody fusion proteins (e.g. Tumor Necrosis Factor Receptor ((TNFR)/Fc domain fusion protein)).

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. Also encompassed within the scope of the term “anti-tumor drug,” are conjugates of peptides with anti-tumor activity, e.g. TNF-α. Conjugates include, but are not limited to those formed between a therapeutic protein and a glycoprotein of the invention. A representative conjugate is that formed between PSGL-1 and TNF-α.

As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diphtheria toxin, and snake venom (e.g., cobra venom).

As used herein, “a radioactive agent” includes any radioisotope that is effective in diagnosing or destroying a tumor. Examples include, but are not limited to, indium-111, cobalt-60. Additionally, naturally occurring radioactive elements such as uranium, radium, and thorium, which typically represent mixtures of radioisotopes, are suitable examples of a radioactive agent. The metal ions are typically chelated with an organic chelating moiety.

Many useful chelating groups, crown ethers, cryptands and the like are known in the art and can be incorporated into the compounds of the invention (e.g., EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, INORGANIC CHEMISTRY IN BIOLOGY AND MEDICINE; Martell, Ed.; American Chemical Society, Washington, D.C., 1980, pp. 279-312; Lindoy, THE CHEMISTRY OF MACROCYCLIC LIGAND COMPLEXES; Cambridge University Press, Cambridge, 1989; Dugas, BIOORGANIC CHEMISTRY; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, MODIFICATION OF PROTEINS: FOOD, NUTRITIONAL, AND PHARMACOLOGICAL ASPECTS;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

As used herein, “administering” means oral administration, administration as a suppository, topical contact, intravenous, intraperitoneal, intramuscular, intralesional, or subcutaneous administration, administration by inhalation, or the implantation of a slow-release device, e.g., a mini-osmotic pump, to the subject. Administration is by any route including parenteral and transmucosal (e.g., oral, nasal, vaginal, rectal, or transdermal), particularly by inhalation. Parenteral administration includes, e.g., intravenous, intramuscular, intra-arteriole, intradermal, subcutaneous, intraperitoneal, intraventricular, and intracranial. Moreover, where injection is to treat a tumor, e.g., induce apoptosis, administration may be directly to the tumor and/or into tissues surrounding the tumor. Other modes of delivery include, but are not limited to, the use of liposomal formulations, intravenous infusion, transdermal patches, etc.

The term “isolated” refers to a material that is substantially or essentially free from components, which are used to produce the material. For peptide conjugates of the invention, the term “isolated” refers to material that is substantially or essentially free from components, which normally accompany the material in the mixture used to prepare the peptide conjugate. “Isolated” and “pure” are used interchangeably. Typically, isolated peptide conjugates of the invention have a level of purity preferably expressed as a range. The lower end of the range of purity for the peptide conjugates is about 60%, about 70% or about 80% and the upper end of the range of purity is about 70%, about 80%, about 90% or more than about 90%.

When the peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, or a similar means).

“Essentially each member of the population,” as used herein, describes a characteristic of a population of peptide conjugates of the invention in which a selected percentage of the modified sugars added to a peptide are added to multiple, identical acceptor sites on the peptide. “Essentially each member of the population” speaks to the “homogeneity” of the sites on the peptide conjugated to a modified sugar and refers to conjugates of the invention, which are at least about 80%, preferably at least about 90% and more preferably at least about 95% homogenous.

“Homogeneity,” refers to the structural consistency across a population of acceptor moieties to which the modified sugars are conjugated. Thus, in a peptide conjugate of the invention in which each modified sugar moiety is conjugated to a site having the same structure as the site to which every other modified sugar is conjugated, the peptide conjugate is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The purity of the peptide conjugates is typically determined by one or more methods known to those of skill in the art, e.g., liquid chromatography-mass spectrometry (LC-MS), matrix assisted laser desorption mass time of flight spectrometry (MALDITOF), capillary electrophoresis, and the like.

“Substantially uniform conjugate” or a “substantially uniform conjugation pattern,” when referring to a glycoconjugate species, refers to the percentage of glycosyl moieties to be acylated that are, in fact, acylated by a selected enzyme. A substantially uniform conjugation pattern exists if substantially all (as defined below) members of a glycosyl group population intended to by acylated are acylated.

The term “substantially” in the above definitions of “substantially uniform” generally means at least about 40%, at least about 70%, at least about 80%, or more preferably at least about 90%, and still more preferably at least about 95% of the acceptor moieties for a particular glycosyltransferase are glycosylated.

### Introduction

The present invention provides conjugates that bear sugars modified with one or more acyl-containing moiety. The sugars can be attached to an amino acid or glycosyl residue of a peptide or glycopeptide, or onto a glycosyl residue of a glycolipid (e.g., sphingosine, ceramide, etc.). Also provided are enymatically-mediated methods for producing the conjugates of the invention. The invention also provides pharmaceutical formulations that include a conjugate formed by a method of the invention.

The conjugates of the invention are formed between a therapeutic core molecule, e.g., glycopeptide, glycolipid, and diverse species such as water-soluble polymers, therapeutic moieties, diagnostic moieties, targeting moieties and the like. Also provided are conjugates that include two or more peptides linked together through a linker arm, i.e., multifunctional conjugates. The multi-functional conjugates of the invention can include two or more copies of the same peptide or a collection of diverse peptides with different structures and/or properties. In exemplary conjugates according to this embodiment, the linker between the two peptides is attached to at least one of the peptides through an acylated glycosyl linking group.

The conjugates of the invention are prepared by the enzymatic conjugation of an activated acyl-containing modifying group to a glycosyl residue, forming a ‘modified sugar’. When the conjugate of the invention is a glycopeptide conjugate, the modified sugar is attached directly to an amino acid of a glycosylation site, or to a glycosyl residue attached either directly or indirectly (e.g., through one or more glycosyl residue) to a glycosylation site.

The modified sugar, when interposed between the peptide (or glycosyl residue) and the modifying group on the sugar becomes what is referred to herein as a “glycosyl linking group,” e.g., “an intact glycosyl linking group.” Using the exquisite selectivity of enzymes, such as proteases, lipases, esterases, acyltransferases, acylases and sugar amidases, the present method provides peptides that bear a desired group at one or more specific locations. Thus, in exemplary conjugates according to the present invention, a modified sugar is attached directly to a selected locus on the peptide chain or, alternatively, the modified sugar is appended onto a carbohydrate moiety of a glycopeptide. Peptides in which modified sugars are bound to both a glycopeptide carbohydrate and directly to an amino acid residue of the peptide backbone are also within the scope of the present invention.

The invention also provides a method for preparing a conjugate of the invention using an activated acyl-containing species. The method includes contacting a glycopeptide, or glycolipid with an activated acyl-containing species and an enzyme for which the activated acyl species is a substrate, which transfers the acyl species onto the glycosyl residue of a peptide or glycolipid.

The methods of the invention, make it possible to assemble modified glycopeptides and glycolipids that have a substantially homogeneous derivatization pattern; the enzymes used in the invention are generally selective for a particular glycosyl residue or for particular substituents, or substituent patterns, on a glycosyl residue. The methods are also practical for large-scale production of modified glycopeptide and glycolipid conjugates. In one embodiment the methods of the invention provide a practical means for large-scale preparation of glycopeptide and glycolipid conjugates having preselected uniform derivatization patterns. The methods are particularly well suited for modification of therapeutic peptides, including but not limited to, glycopeptides that are incompletely glycosylated during production in cell culture cells (e.g., mammalian cells, insect cells, plant cells, fungal cells, yeast cells, or prokaryotic cells) or transgenic plants or animals.

The methods of the invention also provide conjugates of glycosylated and unglycosylated peptides, and glycolipids, with increased therapeutic half-life due to, for example, reduced clearance rate, or reduced rate of uptake by the immune or reticuloendothelial system (RES). Moreover, the methods of the invention provide a means for masking antigenic determinants on peptides, thus reducing or eliminating a host immune response against the peptide. Selective attachment of targeting agents to a peptide or glycolipid using an appropriate modified sugar can also be used to target the peptide or glycolipid to a particular tissue or cell surface receptor that is specific for the particular targeting agent. Moreover, there is provided a class of peptides and glycolipids that are specifically modified with a therapeutic moiety conjugated through a glycosyl linking group.

### The Embodiments

### Compositions

The present invention provides glyco-conjugates in which the sugar moiety is functionalized with a modifying group. The modifying group comprises an acyl group through which the modifying group is conjugated to the sugar moiety. The conjugation is typically achieved through the enzymatically-mediated reaction of an “activated modifying group” with an amine, sulfhydryl, primary hydroxyl, or secondary hydroxyl moiety on the sugar. In an exemplary embodiment, an amine moiety on the sugar is converted to an amide, a urethane or a urea through its reaction with the activated modifying group.

The present invention also provides conjugates in which the modifying group is covalently attached directly to the peptide or the lipid. The conjugation is typically achieved through the enzymatically-mediated reaction of an “activated modifying group” with an amine, sulfhydryl, primary hydroxyl, or secondary hydroxyl moiety on the peptide. In an exemplary embodiment, the “activated modifying groups” can be attached to a side chain of the peptide, such as the hydroxyl group of serine or threonine, the sulfur of cysteine, and/or the amine group of lysine. In an exemplary embodiment, an amine moiety on the peptide is converted to an amide, a urethane or a urea through its reaction with the activated modifying group.

The present invention also provides peptide conjugates in which the peptide is conjugated to a modifying group through a linking group comprising a lipid moiety. In one aspect, the invention provides a peptide conjugate comprising the moiety:

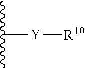

wherein Y is a member selected from O, S and NH, [symbol] represents a connection to the remainder of the conjugate, and R¹⁰ is a member selected from:

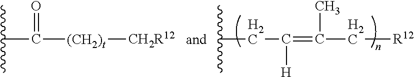

n is an integer from 1 to 20; t is an integer from 1-20; [symbol] represents a connection to Y, and R¹² is a member selected from a water soluble polymer, a water insoluble polymer, a therapeutic moiety, and a diagnostic moiety. In an exemplary embodiment, R¹² is a water soluble polymer. Enzymes useful in the practice of the invention include but are not limited to, wild-type and mutant proteases, lipases, esterases, acylases, acyltransferases, glycosyltransferases, sufotransferases, glycosidases, and the like. In some exemplary embodiments, the enzymes may be wild-type or mutant prenyltransferases (e.g., farnesyltransferases, and geranylgeranyl transferases); N-myristoyltransferases, or palmitoyltransferases.

In an exemplary aspect, the invention provides a lipid or peptide conjugate that includes a glycosyl residue having a structure according to Formula I or Formula II:

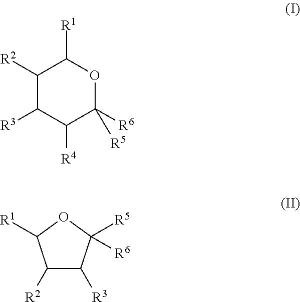

in which the symbols R¹, R², R³, R⁴, R⁵ and R⁶ independently represent H, OR^{7a}, N(R^{7a})₂, SR^{7a}, JC(O)R⁷, substituted or unsubstituted alkyl, substituted or unsubstituted heteroalkyl, substituted or unsubstituted aryl, substituted or unsubstituted heteroaryl or substituted or unsubstituted heterocycloalkyl. The symbol J represents a bond, O, S or NH. The symbol R⁷ represents H, R⁸, OR⁸, NR⁸R⁹, substituted or unsubstituted alkyl, substituted or unsubstituted heteroalkyl, substituted or unsubstituted aryl, substituted or unsubstituted heteroaryl, or substituted or unsubstituted heterocycloalkyl. Each R^{7a} is independently selected and represents H, substituted or unsubstituted alkyl, substituted or unsubstituted heteroalkyl, substituted or unsubstituted aryl, substituted or unsubstituted heteroaryl, or substituted or unsubstituted heterocycloalkyl.

R⁸ and R⁹ independently represent H, substituted or unsubstituted alkyl, substituted or unsubstituted heteroalkyl, substituted or unsubstituted aryl, substituted or unsubstituted heteroaryl, or substituted or unsubstituted heterocycloalkyl.

In the conjugates of the invention, at least one of R¹, R², R³, R⁴, R⁵ and R⁶ comprises a modifying group (e.g., polymer, water-soluble polymer, therapeutic moiety, etc.) as discussed herein. The modifying group is linked to the glycosyl residue through a moiety that includes a carbonyl group, e.g., an acyl group (e.g., ROC(O)R′). In an exemplary embodiment, one of R⁸ or R⁹ is a water-soluble polymer moiety (e.g., m-PEG, branched m-PEG).

The symbol R⁶ represents an amino acid residue of the peptide, a carbohydrate linker moiety covalently bound to an amino acid residue of the peptide, and combinations thereof.

Alternatively, when the conjugate includes a lipid, R⁶ represents an aglycone, a carbohydrate linker moiety covalently bound to an aglycone, and combinations thereof.

When R⁶ is a carbohydrate linker moiety, exemplary moieties bound to the glycosyl core shown in Formula I include Gal, GalNAc, Man, GlcNAc, Fuc and Sia, and Glu. Those of skill will appreciate that the carbohydrate linker moiety can include these, and other, carbohydrate residues in essentially any combination and sequence.

In another exemplary embodiment, at least one of R¹, R², R³, R⁴, R⁵ and R⁶ is a member selected from:

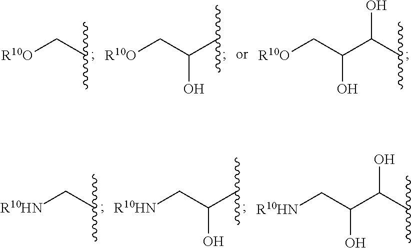

wherein [symbol] represents a connection to the remainder of the conjugate, R¹⁰ includes a modifying group attached through a moiety that includes an acyl group. Exemplary polymeric modifying groups include a poly(ether), a poly(sialic acid), and a poly(amino acid), e.g., poly(aspartic acid), poly(glutamic acid).

In a further exemplary embodiment, R¹⁰ can be H. In another exemplary embodiment, R¹⁰ can comprise a modifying group. In another exemplary embodiment, R¹⁰ can have a structure according to the formula:

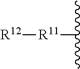

in which the symbol R¹¹ represents a linker joining O to R¹² or N to R¹² and [symbol] represents a connection to either the O or the N of the remainder of the conjugate. Exemplary linkers are members selected from substituted or unsubstituted alkyl and substituted or unsubstituted heteroalkyl moieties. R¹² is a modifying group.

The linker is of any structure appropriate to join O and R¹² or N and R¹² with a level of stability appropriate for a selected application. In an exemplary embodiment in which R¹² is a water-soluble polymer, e.g., m-PEG, the linker is a substituted or unsubstituted alkyl or substituted or unsubstituted heteroalkyl moiety that has an acyl moiety attached, as a linking moiety, to O or to N. An exemplary acyl-containing linking moiety is —C(O)NH, affording a R¹¹ moiety that is attached to the remainder of the saccharide through a urethane linkage. Similarly, when the modifying group is a water-soluble polymer, the polymer can be joined to R¹¹ through a linking moiety such as an amide or a urethane. The art-relevant for cross-linking two molecular species is well developed and it is within the abilities of one of skill in the art to identify an appropriate R¹¹ moiety and a precursor to this moiety.

In one embodiment, the present invention provides a conjugate comprising the moiety:

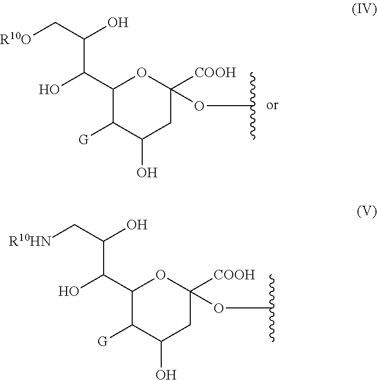

wherein [symbol] represents a connection to the remainder of the conjugate, R¹⁰ is a member selected from H and R¹²—R¹¹—; G is a member selected from HO, R¹²—R¹¹—O—, NH₂ and R¹²—R¹¹—NH— and —C(O)(C₁-C₆)alkyl; R¹² is a modifying group, such as a straight-chain or branched poly(ethylene glycol) residue; and R¹¹ represents a linker joining O to R¹² or N to R¹², e.g., a bond (“zero order”), substituted or unsubstituted alkyl and substituted or unsubstituted heteroalkyl. In exemplary embodiments, when the conjugate is according to Formula (IV), R¹⁰ is H, G is R¹²—R¹¹—O—, R¹²—R¹¹—NH— and when G is —C(O)(C₁-C₆)alkyl, R¹⁰ is R¹²—R¹¹—.

In another exemplary embodiment, the invention provides a conjugate formed between a modified sugar of the invention and a lipid or peptide. In this embodiment, the sugar moiety of the modified sugar becomes a glycosyl linking group interposed between the lipid or peptide substrate and the modifying group. An exemplary glycosyl linking group is an intact glycosyl linking group, in which the glycosyl moiety or moieties forming the linking group are not degraded by chemical (e.g., sodium metaperiodate) or enzymatic (e.g., oxidase) processes. Selected conjugates of the invention include a modifying group that is attached to the amine moiety of an amino-saccharide, e.g., mannosamine, glucosamine, galactosamine, sialic acid etc. Exemplary modifying group-intact glycosyl linking group cassettes according to this motif are based on a sialic acid structure, such as those having the formulae:

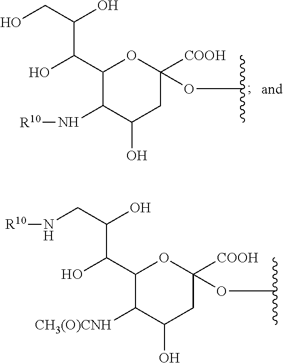

In the formulae above, R¹⁰ is as described above. [symbol] represents a connection to the remainder of the conjugate.

In still a further exemplary embodiment, the conjugate is formed between a lipid or peptide substrate and a glycosyl moiety in which the modifying group is attached through a linker at the 6-carbon position of the glycosyl moiety. Thus, illustrative conjugates according to this embodiment have the formula:

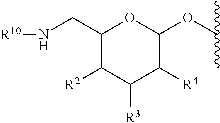

in which the radicals are as discussed above. Such glycosyl moieties include, without limitation, glucose, glucosamine, N-acetyl-glucosamine, galactose, galactosamine, N-acetyl-galactosamine, mannose, mannosamine, N-acetyl-mannosamine, and the like. [symbol] represents a connection to the remainder of the conjugate.

Due to the versatility of the methods available for modifying glycosyl residues on a therapeutic lipid or peptide, the glycosyl structures on the lipid or peptide conjugates of the invention can have substantially any structure. Moreover, the glycans can be O-linked or N-linked. As exemplified in the discussion below, each of the pyranose and furanose derivatives discussed above can be a component of a glycosyl moiety of a lipid or peptide.

The invention provides a modified lipid or peptide that includes a glycosyl group having the formula:

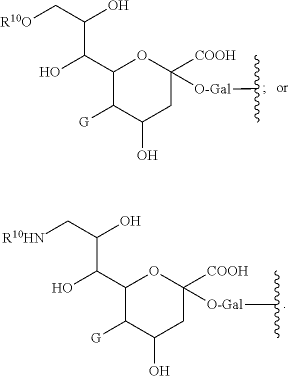

In other embodiments, the group has the formula:

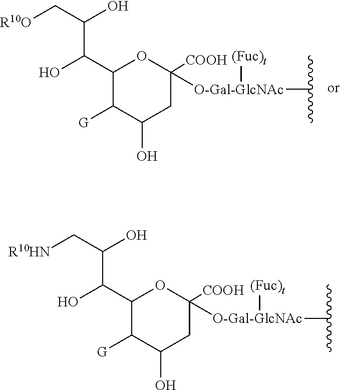

in which the index t is 0 or 1.

In a still further exemplary embodiment, the group has the formula:

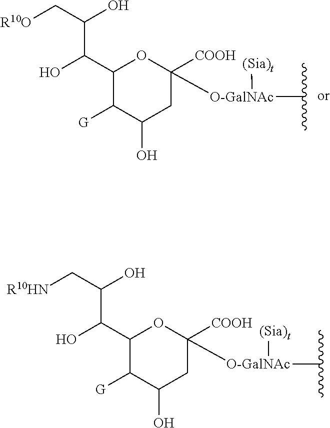

in which the index t is 0 or 1.

In yet another embodiment, the group has the formula:

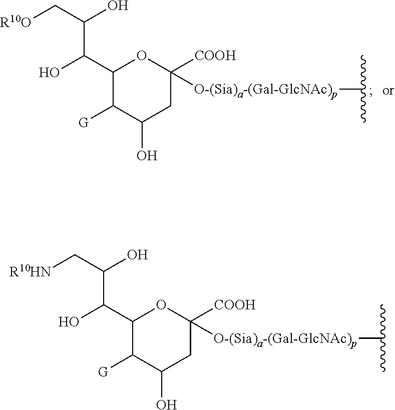

in which the index p represents an integer from 1 to 10; and a is either 0 or 1.

In an exemplary embodiment, the conjugate has a structure according to the following formula:

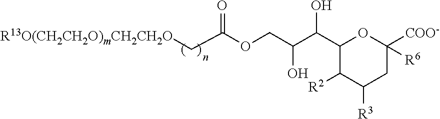

in which the index m is an integer from 1 to 2500, the index n is an integer from 0 to 40 and R¹³ is a member selected from H and substituted or unsubstituted alkyl.

In an exemplary embodiment, the conjugate has a structure according to the following formula:

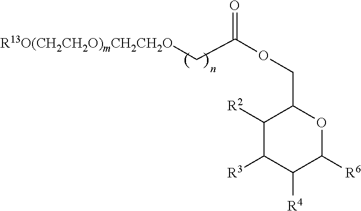

in which the index m is an integer from 1 to 2500, the index n is an integer from 0 to 40; and R¹³ is a member selected from H and substituted or unsubstituted alkyl.

In an exemplary embodiment, a glycoPEGylated lipid or peptide conjugate of the invention includes at least one N-linked glycosyl residue selected from the glycosyl residues set forth below:

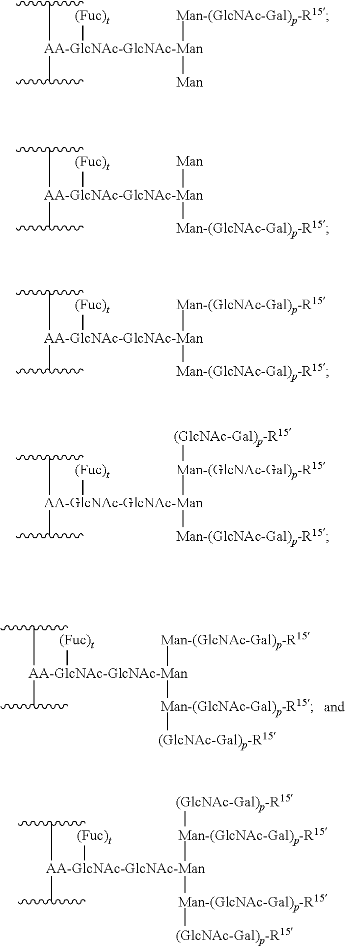

In the formulae above, the index t is 0 or 1 and the index p is an integer from 1 to 10, and ‘AA’ represents an amino acid of the peptide. [symbol] represents a connection to the remainder of the peptide. The symbol R^{15′} represents H, OH (e.g., Gal-OH), a sialyl moiety, a polymer modified sialyl moiety (i.e., glycosyl linking group-polymeric modifying moiety (Sia-L-R¹)) or a sialyl moiety to which is bound a polymer modified sialyl moiety (e.g., Sia-Sia-L-R¹) (“Sia-Sia^{p}”). Exemplary polymer modified glycosyl moieties have a structure according to Formulae I and II. An exemplary lipid or peptide conjugate of the invention will include at least one glycan having a R^{15′} that includes a structure according to Formulae I or II. The oxygen, with the open valence, of Formulae I and II is preferably attached through a glycosidic linkage to a carbon of a Gal or GalNAc moiety. In a further exemplary embodiment, the oxygen is attached to the carbon at position 3 of a galactose residue. In an exemplary embodiment, the modified sialic acid is linked α2,3-to the galactose residue. In another exemplary embodiment, the sialic acid is linked α2,6-to the galactose residue.

In another exemplary embodiment, the invention provides a lipid or peptide conjugate that includes a glycosyl linking group, such as those set forth above, that is covalently attached to an amino acid residue of the peptide. In one embodiment according to this motif, the glycosyl linking moiety is linked to a galactose residue through a Sia residue:

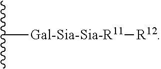

An exemplary species according to this motif is prepared by conjugating Sia-R¹¹-R¹² to a terminal sialic acid of a glycan using an enzyme that forms Sia-Sia bonds, e.g., CST-II, ST8Sia-II, ST8Sia-III and ST8Sia-IV.

In another exemplary embodiment, the glycans have a formula that is selected from the group:

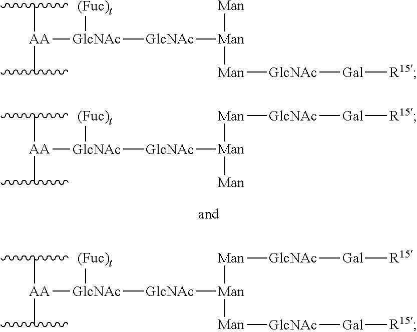

and combinations thereof.

The glycans of this group generally correspond to those found on a lipid or peptide conjugate that is produced by insect (e.g., Sf-9) cells, following remodeling according to the methods set forth herein. For example insect-derived lipid or peptide that is expressed with a tri-mannosyl core is subsequently contacted with a GlcNAc donor and a GlcNAc transferase and a Gal donor and a Gal transferase. Appending GlcNAc and Gal to the tri-mannosyl core is accomplished in either two steps or a single step. A modified sialic acid is added to at least one branch of the glycosyl moiety as discussed herein. Those Gal moieties that are not functionalized with the modified sialic acid are optionally “capped” by reaction with a sialic acid donor in the presence of a sialyl transferase.

In an exemplary embodiment, at least 60% of terminal Gal moieties in a population of peptides is capped with sialic acid, preferably at least 70%, more preferably, at least 80%, still more preferably at least 90% and even more preferably at least 95%, 96%, 97%, 98% or 99% are capped with sialic acid.

In each of the formulae above, R^{15′} is as discussed above. Moreover, an exemplary modified lipid or peptide of the invention will include at least one glycan with an R^{15′} moiety having a structure according to Formulae I or II.

In an exemplary embodiment, the glycosyl linking moiety has the formula:

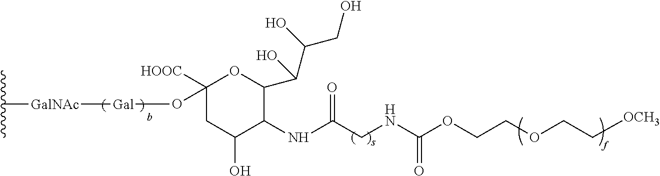

in which the index b is 0 or 1. [symbol] represents a connection to the remainder of the conjugate. The index s represents an integer from 1 to 10; and the index f represents an integer from 1 to 2500. Generally preferred is the use of a PEG moiety that has a molecular weight of about 20 kDa.

In another exemplary embodiment, the lipid or peptide is derived from insect cells, remodeled by adding GlcNAc and Gal to the mannose core and glycopegylated using a sialic acid bearing a linear PEG moiety, affording a lipid or peptide that comprises at least one moiety having the formula:

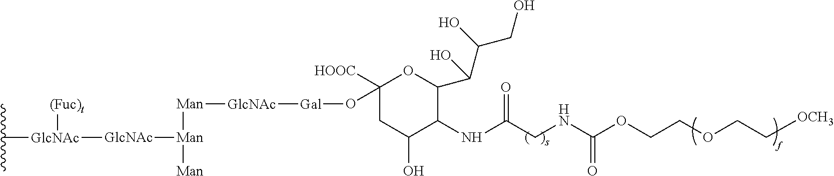

in which the index s represents an integer from 1 to 10; the index f represents an integer from 1 to 2500; and [symbol] represents a connection to the remainder of the conjugate.

As discussed herein, R¹⁰ can comprise a linear or a branched modifying group, thus forming a linear or branched conjugate of the invention. An exemplary precursor of use to form the branched conjugates according to this embodiment of the invention has a structure according to Formulae IIIa or IIIb:

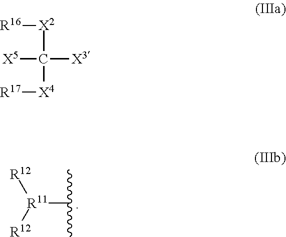

The branched polymer species according to this formula are essentially pure water-soluble polymers. X^{3′} is a moiety that includes an ionizable, e.g., OH, COOH, H₂PO₄, HSO₃, HPO₃, and salts thereof, etc.) or other reactive functional group, e.g., infra. C is carbon. X⁵ is preferably a non-reactive group (e.g., H, unsubstituted alkyl, unsubstituted heteroalkyl), and can be a polymeric arm. R¹⁶ and R¹⁷ are independently selected polymeric arms/modifying groups, e.g., nonpeptidic, nonreactive polymeric arms (e.g., PEG)). X² and X⁴ are linkage fragments that are preferably essentially non-reactive under physiological conditions, which may be the same or different. An exemplary linker includes neither aromatic nor ester moieties. Alternatively, these linkages can include one or more moiety that is designed to degrade under physiologically relevant conditions, e.g., esters, disulfides, etc. X² and X⁴ join polymeric arms R¹⁶ and R¹⁷ to C. When X^{3′} is reacted with a reactive functional group of complementary reactivity on a linker, sugar or linker-sugar cassette, X^{3′} is converted to a component of linkage fragment X³. R¹¹ is as described above. Each R¹² is independently selected as described above.

Exemplary linkage fragments for X², X³ and X⁴ are independently selected and include S, SC(O)NH, HNC(O)S, SC(O)O, O, NH, NHC(O), (O)CNH and NHC(O)O, and OC(O)NH, CH₂S, CH₂O, CH₂CH₂O, CH₂CH₂S, (CH₂)ₒ, (CH₂)ₒS or (CH₂)ₒY′-PEG wherein, Y′ is S, NH, NHC(O), C(O)NH, NHC(O)O, OC(O)NH, or O and the index o is an integer from 1 to 50. In an exemplary embodiment, the linkage fragments X² and X⁴ are different linkage fragments.

In an exemplary embodiment, the precursor (III), or an activated derivative thereof, is reacted with, and thereby bound to a sugar, an activated sugar or a sugar nucleotide through a reaction between X^{3′} and a group of complementary reactivity on the sugar moiety, e.g., an amine. Alternatively, X^{3′} reacts with a reactive functional group on a precursor to R¹¹. One or more of R¹, R², R³, R⁴, R⁵ or R⁶ of Formulae I and II can include the branched polymeric modifying moiety, or this moiety bound through R¹¹.

In an exemplary embodiment, the moiety:

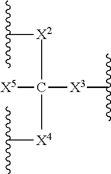

is R¹¹. In this embodiment, an exemplary linker is derived from a natural or unnatural amino acid, amino acid analogue or amino acid mimetic, or a small peptide formed from one or more such species. For example, certain branched polymers found in the compounds of the invention have the formula:

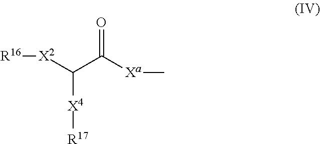

X^{a} is a linkage fragment that is formed by the reaction of a reactive functional group, e.g., X^{3′}, on a precursor of the branched polymeric modifying moiety and a reactive functional group on the sugar moiety, or a precursor to a linker. For example, when X^{3′} is a carboxylic acid, it can be activated and bound directly to an amine group pendent from an amino-saccharide (e.g., Sia, GalNH₂, GlcNH₂, ManNH₂, etc.), forming a X^{a} that is an amide. Additional exemplary reactive functional groups and activated precursors are described hereinbelow. The index c represents an integer from 1 to 10. The other symbols have the same identity as those discussed above.

In another exemplary embodiment, X^{a} is a linking moiety formed with another linker:

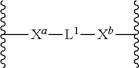

in which X^{b} is a second linkage fragment and is independently selected from those groups set forth for X^{a}, and, similar to R¹¹, L¹ is a bond, substituted or unsubstituted alkyl or substituted or unsubstituted heteroalkyl.

Exemplary species for X^{a} and X^{b} include S, SC(O)NH, HNC(O)S, SC(O)O, O, NH, NHC(O), C(O)NH and NHC(O)O, and OC(O)NH.

In another exemplary embodiment, X⁴ is a peptide bond to R¹⁷, which is an amino acid, di-peptide (e.g., Lys-Lys) or tri-peptide (E.G., Lys-Lys-Lys) in which the alpha-amine moiety(ies) and/or side chain heteroatom(s) are modified with a polymeric modifying moiety.

In a further exemplary embodiment, the conjugates of the invention include a moiety, e.g., an R^{15′} moiety that has a formula that is selected from:

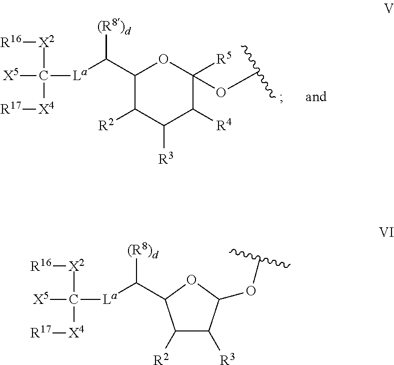

in which the identity of the radicals represented by the various symbols is the same as that discussed hereinabove. L^{a} is a bond or a linker as discussed above for R¹¹ and L¹, e.g., substituted or unsubstituted alkyl or substituted or unsubstituted heteroalkyl moiety. In an exemplary embodiment, L^{a} is a moiety of the side chain of sialic acid that is functionalized with the polymeric modifying moiety as shown. Exemplary L^{a} moieties include substituted or unsubstituted alkyl chains that include one or more OH or NH₂.

In yet another exemplary embodiment, the invention provides conjugates having a moiety, e.g., an R^{15′} moiety with formula:

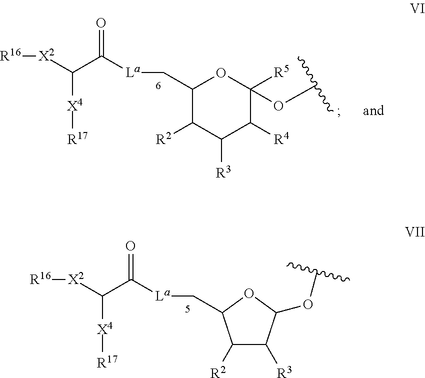

The identity of the radicals represented by the various symbols is the same as that discussed hereinabove. As those of skill will appreciate, the linker arm in Formulae VI and VII is equally applicable to other modified sugars set forth herein. In exemplary embodiment, the species of Formulae VI and VII are the R^{15′} moieties attached to the glycan structures set forth herein.

In yet another exemplary embodiment, the lipid or peptide includes an R^{15′} moiety with the formula:

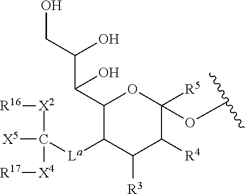

in which the identities of the radicals are as discussed above. An exemplary species for L^{a} is —(CH₂)ⱼC(O)NH(CH₂)ₕC(O)NH—, in which h and j are independently selected integers from 0 to 10. A further exemplary species is —C(O)NH—.

The embodiments of the invention set forth above are further exemplified by reference to species in which the polymer is a water-soluble polymer, particularly poly(ethylene glycol) (“PEG”), e.g., methoxy-poly(ethylene glycol). Those of skill will appreciate that the focus in the sections that follow is for clarity of illustration and the various motifs set forth using PEG as an exemplary polymer are equally applicable to species in which a polymer other than PEG is utilized.

PEG of any molecular weight, e.g., 1 kDa, 2 kDa, 5 kDa, 10 kDa, 15 kDa, 20 kDa, 30 kDa and 40 kDa is of use in the present invention.

In an exemplary embodiment, the R^{15′} moiety has a formula that is a member selected from the group:

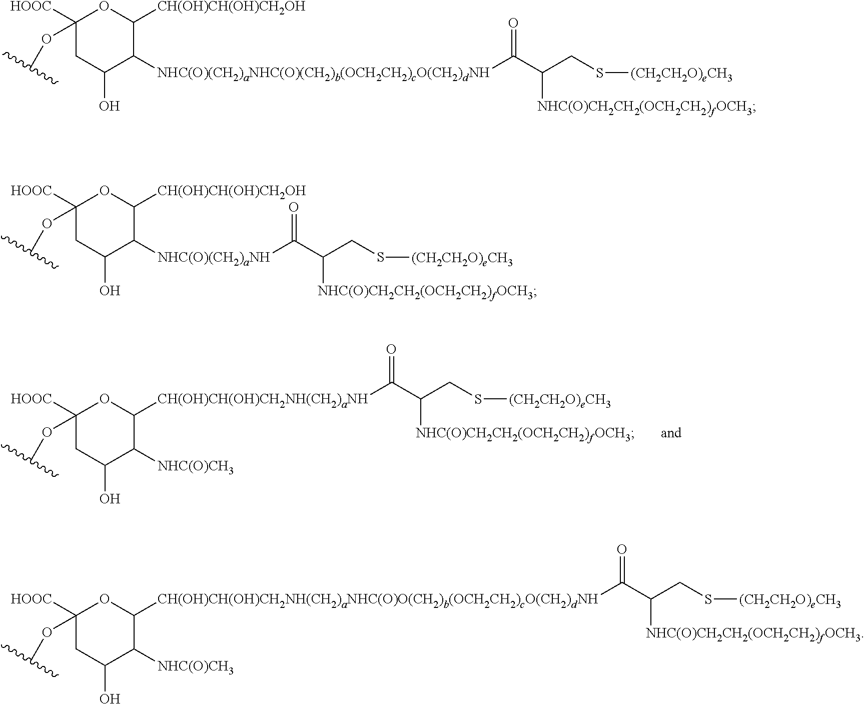

In each of the structures above, the linker fragment NH(CH₂)ₐ— can be present or absent.

In other exemplary embodiments, the lipid or peptide conjugate includes an R^{15′} moiety selected from the group:

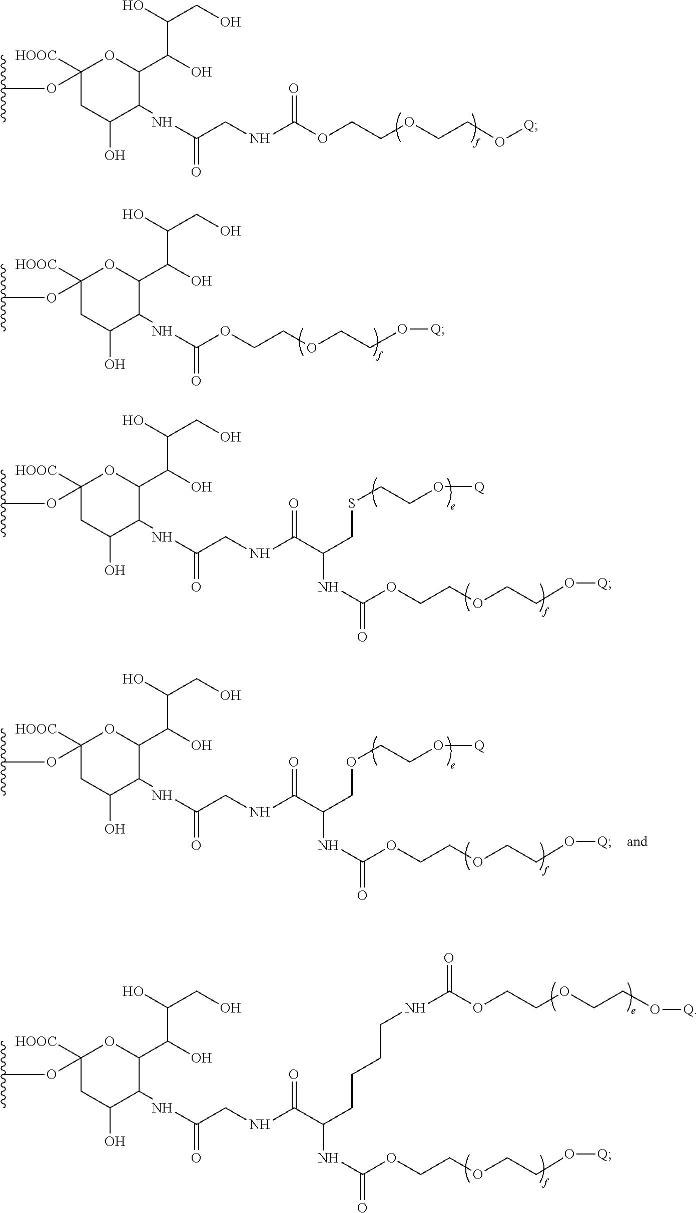

In each of the formulae above, the indices e and f are independently selected from the integers from 1 to 2500. In further exemplary embodiments, e and f are selected to provide a PEG moiety that is about 1 kD, 2 kD, 10 kD, 15 kD, 20 kD, 30 kD or 40 kD. The symbol Q represents substituted or unsubstituted alkyl (e.g., C₁-C₆ alkyl, e.g., methyl), substituted or unsubstituted heteroalkyl or H.

Other branched polymers have structures based on di-lysine (Lys-Lys) peptides, e.g.:

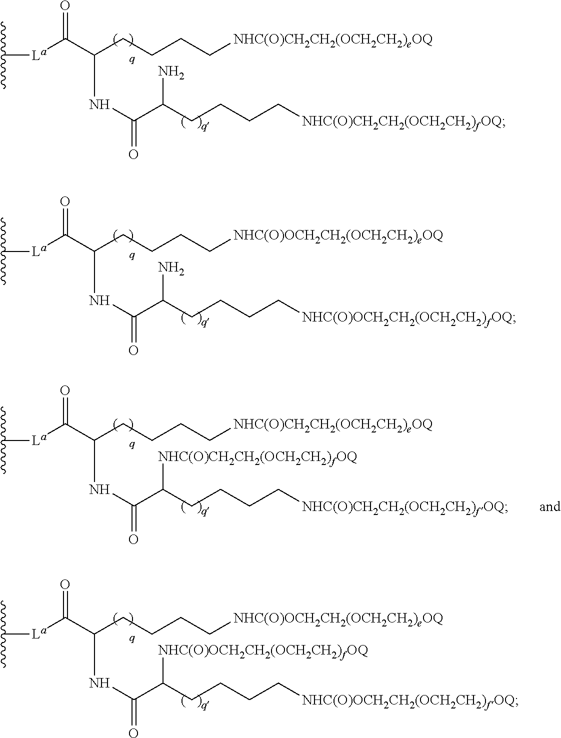

and tri-lysine peptides (Lys-Lys-Lys), e.g.:

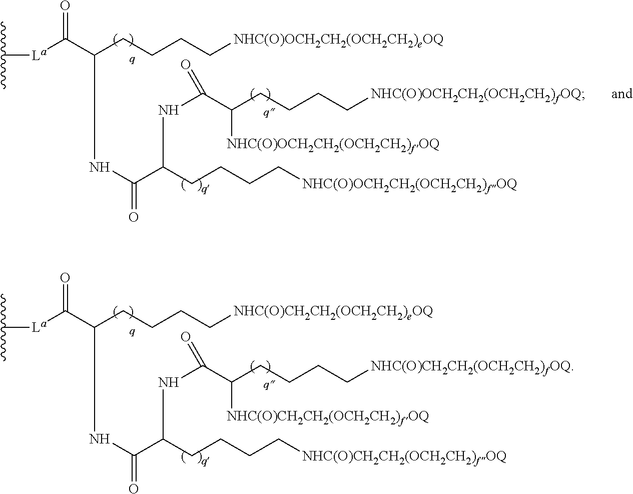

In each of the figures above, the indices e, f, f′ and f″ represent integers independently selected from 1 to 2500. The indices q, q′ and q″ represent integers independently selected from 1 to 20.

In another exemplary embodiment, the lipid or peptide comprises a glycosyl moiety selected from the formulae:

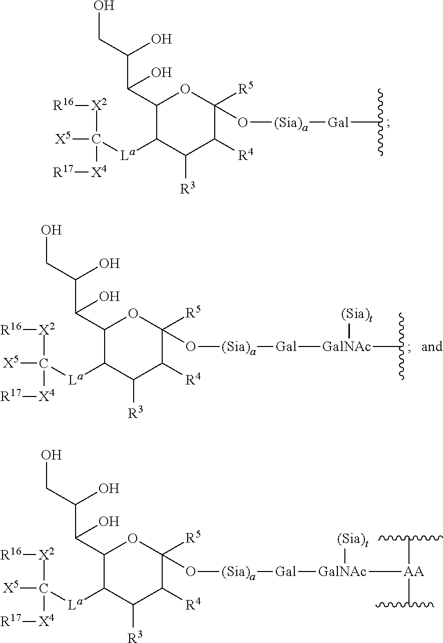

in which L^{a} is a bond or a linker as described herein; the index t represents 0 or 1; and the index a represents 0 or 1. Each of these groups can be included as components of the mono-, bi-, tri- and tetra-antennary saccharide structures set forth above.

In yet another embodiment, the lipid or peptide conjugates of the invention include a modified glycosyl residue that includes the substructure selected from:

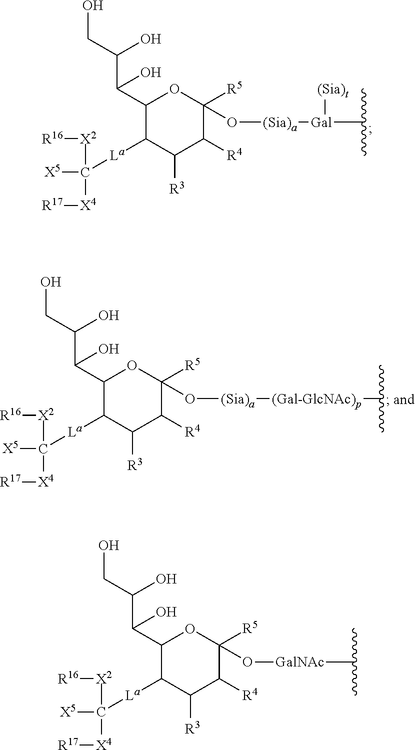

in which the index a and the linker L^{a} are as discussed above. The index p is an integer from 1 to 10. The indices t and a are independently selected from 0 or 1. Each of these groups can be included as components of the mono-, bi-, tri- and tetra-antennary saccharide structures set forth above.

In a further exemplary embodiment, the invention utilizes modified sugars in which the 6-hydroxyl position is converted to the corresponding amine moiety, which bears a linker-modifying group cassette such as those set forth above. Exemplary glycosyl groups that can be used as the core of these modified sugars include Gal, GalNAc, Glc, GlcNAc, Fuc, Xyl, Man, and the like. A representative modified sugar according to this embodiment has the formula:

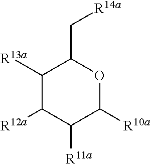

in which R^{11a}, R^{12a}, R^{13a} and R^{14a} are members independently selected from H, OH, C(O)CH₃, NH, and NH C(O)CH₃. R^{10a} is a link to another glycosyl residue (—O-glycosyl) or to an amino acid of the peptide (—NH-(peptide)). R^{14a} is OR¹⁰ or NHR¹⁰. R¹⁰ is described above.

Selected conjugates according to this motif are based on mannose, galactose or glucose, or on species having the stereochemistry of mannose, galactose or glucose. The general formulae of these conjugates are:

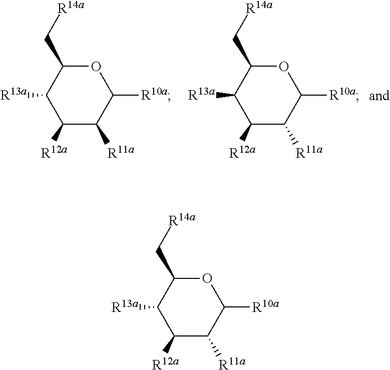

As discussed above, the invention provides saccharides bearing a modifying group, activated analogues of these species and conjugates formed between species such as peptides and lipids and a modified saccharide of the invention.

Still further exemplary species of use in the invention are substrates for proteases. Thus, it is within the scope of the invention to utilize of any one or more of the structures shown above, or analogues thereof, to form a conjugate of the invention. See, for example, WO 03/014371.

As discussed above, selected conjugates of the invention include one or more polymer moiety, such as PEG. Conjugates of the invention that include modifying groups that are PEG can be of the formula:

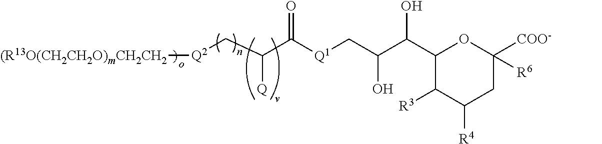

in which, the index “m” is an integer from 1 to 2500; the index n represents an integer from 0 to 40; and the symbol R¹³ represents H or substituted or unsubstituted alkyl. Q is H, substituted or unsubstituted alkyl, or a side chain of an amino acid, or a linker to a polymer. Q¹ is selected from O, S and species that include a nitrogen atom. Q² is O, S or NH. Alternatively, Q² is an amino acid or peptidyl residue. The index “o” is an integer from 1 to 4. When o is greater than 1, Q² is generally an amino acid or peptidyl residue as discussed herein and the PEG moiety is optionally a branched PEG moiety. The index v is either 0 or 1. In an exemplary conjugate according to this formula, the glycosyl residue is a sialic acid residue.

Yet another exemplary PEG conjugate has the formula:

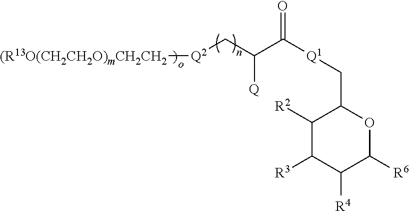

in which the index m is an integer from 1 to 2500; the index n is an integer from 0 to 40; and R¹³ is a member selected from H and substituted or unsubstituted alkyl. Q, Q′ and Q² are as discussed above. Exemplary Q² species are linkers, e.g., urethane, amide, amino acid residue, peptidyl residue and the like.

The components of the conjugates of the invention are discussed in greater detail in the sections that follow.

### Sugars

Any sugar can be utilized as the sugar core of the conjugates of the invention. Exemplary sugar cores that are useful in forming the compositions of the invention include, but are not limited to, glucose, galactose, and mannose and N-acetyl analogues of these sugars. Also of use are fucose, xylose, ribose, arabinose, and sialic acid. Also encompassed within the invention are species in which the sugar core is a disaccharide, an oligosaccharide or a polysaccharide. The sugar core can also be attached to an aglycone, such as a peptide, a lipid, or an sugar nucleotide (such as cytosine monophosphate (CMP), uracil diphosphate (UDP), and guanosine diphosphate (GDP)).

### Modifying Groups and Activated Modifying Groups

Another glyco-conjugate component is the modifying group. Prior to the enzymatic reaction, these groups are covalently attached to a leaving group, thus forming an activated modifying group. In an exemplary embodiment, the activated modifying group has the structure:

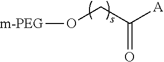

In which the index “s” represents an integer from 1 to 20; and A is any activating group that can be removed by one or more lipase, protease, acyltransferase, esterase or acylase in the process of transferring the acyl donor moiety to the saccharide. A is an activating group, such as a leaving group. Exemplary leaving groups include allyl groups, CH₂CH═CH₂, O-aryl, active esters, S-alkyl, S-aryl and the like. Through the enzyme catalyzed reaction, the leaving group of the activated modifying group is displaced, and a covalent bond is formed between the sugar moiety and the modifying group, thus forming the glyco-conjugate.

### Modifying Groups

The modifying groups of the invention can be any group, e.g., water-soluble polymer, water-insoluble polymer, therapeutic moiety, etc., that can be conjugated to a sugar moiety through the use of the enzymes described herein.

### Water-Soluble Polymers

Many water-soluble polymers are known to those of skill in the art and are useful in practicing the present invention. The term water-soluble polymer encompasses species such as saccharides (e.g., dextran, amylose, hyalouronic acid, poly(sialic acid), heparans, heparins, etc.); poly(amino acids), e.g., poly(aspartic acid) and poly(glutamic acid); nucleic acids; synthetic polymers (e.g., poly(acrylic acid), poly(ethers), e.g., poly(ethylene glycol); peptides, proteins, and the like. The present invention may be practiced with any water-soluble polymer with the sole limitation that the polymer must include a point at which the remainder of the conjugate can be attached.

Methods for activation of polymers can also be found in WO 94/17039, U.S. Pat. No. 5,324,844, WO 94/18247, WO 94/04193, U.S. Pat. No. 5,219,564, U.S. Pat. No. 5,122,614, WO 90/13540, U.S. Pat. No. 5,281,698, and more WO 93/15189, and for conjugation between activated polymers and peptides, e.g. Coagulation Factor VIII (WO 94/15625), hemoglobin (WO 94/09027), oxygen carrying molecule (U.S. Pat. No. 4,412,989), ribonuclease and superoxide dismutase (Veronese at al., App. Biochem. Biotech. 11: 141-45 (1985)).

Preferred water-soluble polymers are those in which a substantial proportion of the polymer molecules in a sample of the polymer are of approximately the same molecular weight; such polymers are “homodisperse.”

The present invention is further illustrated by reference to a poly(ethylene glycol) conjugate. Several reviews and monographs on the functionalization and conjugation of PEG are available. See, for example, Harris, Macronol. Chem. Phys. C25: 325-373 (1985); Scouten, Methods in Enzymology 135: 30-65 (1987); Wong et al., Enzyme Microb. Technol. 14: 866-874 (1992); Delgado et al., Critical Reviews in Therapeutic Drug Carrier Systems 9: 249-304 (1992); Zalipsky, Bioconjugate Chem. 6: 150-165 (1995); and Bhadra, et al., Pharmazie, 57:5-29 (2002). Routes for preparing reactive PEG molecules and forming conjugates using the reactive molecules are known in the art. For example, U.S. Pat. No. 5,672,662 discloses a water soluble and isolatable conjugate of an active ester of a polymer acid selected from linear or branched poly(alkylene oxides), poly(oxyethylated polyols), poly(olefinic alcohols), and poly(acrylomorpholine).

U.S. Pat. No. 6,376,604 sets forth a method for preparing a water-soluble 1-benzotriazolylcarbonate ester of a water-soluble and non-peptidic polymer by reacting a terminal hydroxyl of the polymer with di(1-benzotriazoyl)carbonate in an organic solvent. The active ester is used to form conjugates with a biologically active agent such as a protein or peptide.

WO 99/45964 describes a conjugate comprising a biologically active agent and an activated water soluble polymer comprising a polymer backbone having at least one terminus linked to the polymer backbone through a stable linkage, wherein at least one terminus comprises a branching moiety having proximal reactive groups linked to the branching moiety, in which the biologically active agent is linked to at least one of the proximal reactive groups. Other branched poly(ethylene glycols) are described in WO 96/21469, U.S. Pat. No. 5,932,462 describes a conjugate formed with a branched PEG molecule that includes a branched terminus that includes reactive functional groups. The free reactive groups are available to react with a biologically active species, such as a protein or peptide, forming conjugates between the poly(ethylene glycol) and the biologically active species. U.S. Pat. No. 5,446,090 describes a bifunctional PEG linker and its use in forming conjugates having a peptide at each of the PEG linker termini.

Conjugates that include degradable PEG linkages are described in WO 99/34833; and WO 99/14259, as well as in U.S. Pat. No. 6,348,558. Such degradable linkages are applicable in the present invention.

Although both reactive PEG derivatives and conjugates formed using the derivatives are known in the art, until the present invention, it was not recognized that a conjugate could be formed selectively between a specific site on a glycopeptide or glycolipid and PEG (or other polymer) through an intact glycosyl linking group.

In another exemplary embodiment, poly(ethylene glycol) molecules of use in the invention include, but are not limited to, those species set forth below.

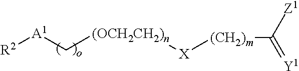

in which R² is H, substituted or unsubstituted alkyl, substituted or unsubstituted aryl, substituted or unsubstituted heteroaryl, substituted or unsubstituted heterocycloalkyl, substituted or unsubstituted heteroalkyl, e.g., acetal, OHC—, H₂N—(CH₂)_{q}—, HS—(CH₂)_{q}, and —(CH₂)_{q}C(Y¹)Z²; -sugar-nucleotide, or protein. The index “n” represents an integer from 1 to 2500. The indeces m, o, and q independently represent integers from 0 to 20. The symbols Z′ and Z² independently represent OH, NH₂, halogen, S—R³, the alcohol portion of activated esters, —(CH₂)ₚC(Y²)V, —(CH₂)ₚU(CH₂)ₛC(Y²)ᵥ, sugar-nucleotide, protein, and leaving groups, e.g., imidazole, p-nitrophenyl, HOBT, tetrazole, halide. The symbols X, Y¹, Y², A¹, and U independently represent the moieties O, S, N—R⁴. The symbol V represents OH, NH₂, halogen, S—R⁵, the alcohol component of activated esters, the amine component of activated amides, sugar-nucleotides, and proteins. The indices p, q, s and v are members independently selected from the integers from 0 to 20. The symbols R³, R⁴ and R⁵ independently represent H, substituted or unsubstituted alkyl, substituted or unsubstituted heteroalkyl, substituted or unsubstituted aryl, substituted or unsubstituted heterocycloalkyl and substituted or unsubstituted heteroaryl.

In other exemplary embodiments, the poly(ethylene glycol) molecule is selected from the following:

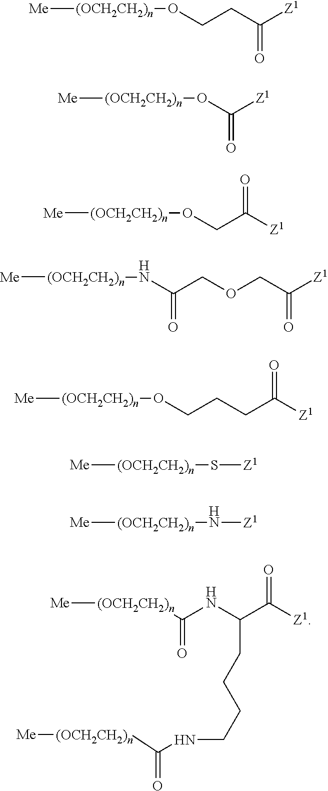

The poly(ethylene glycol) useful in forming the conjugate of the invention is either linear or branched. Branched poly(ethylene glycol) molecules suitable for use in the invention include, but are not limited to, those described by the following formula:

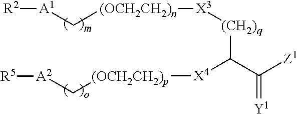

in which R² and R⁵ are members independently selected from the groups defined for R², above. A¹ and A² are members independently selected from the groups defined for A¹, above. The indices m, n, o, p and q are as described above. Z¹ and Y¹ are as described above. X³ and X⁴ are members independently selected from S, SC(O), O, NH, NHC(O) and NHC(O)O.

In other exemplary embodiments, the branched PEG is based upon a cysteine, serine di-lysine or tri-lysine core. Thus, further exemplary branched PEGs include:

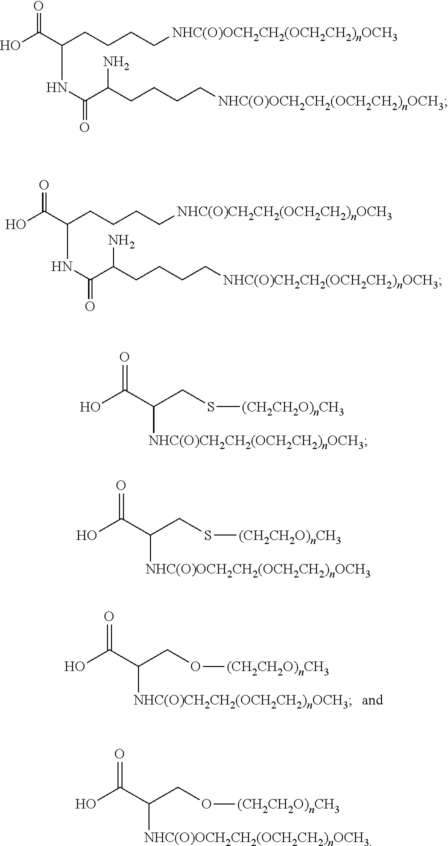

In exemplary embodiments of the invention, the PEG is m-PEG (5 kD, 10 kD, or 20 kD). An exemplary branched PEG species is a serine- or cysteine-(m-PEG)₂ in which the m-PEG is a 20 kD m-PEG.

As will be apparent to those of skill, the branched polymers of use in the invention include variations on the themes set forth above. For example the di-lysine-PEG conjugate shown above can include three polymeric subunits, the third bonded to the α-amine shown as unmodified in the structure above. Similarly, the use of a tri-lysine functionalized with three or four polymeric subunits is within the scope of the invention.

Those of skill in the art will appreciate that one or more of the mPEG arms of the branched polymer can be replaced by a PEG moiety with a different terminus, e.g., OH, COOH, NH₂, C₂-C₁₀-alkyl, etc. Moreover, the structures above are readily modified by inserting alkyl linkers (or removing carbon atoms) between the α-carbon atom and the functional group of the side chain. Thus, “homo” derivatives and higher homologues, as well as lower homologues are within the scope of cores for branched PEGs of use in the present invention. Furthermore, one or more PEG moiety can be replaced by a modifying group other than a water-soluble polymer, e.g., therapeutic moiety, biomolecule, or water-insoluble polymer.

### Water-Insoluble Polymers

In another embodiment, analogous to those discussed above, the modifying group is a water-insoluble polymer, rather than a water-soluble polymer. The glyco-conjugates of the invention may also include one or more water-insoluble polymers. This embodiment of the invention is illustrated by the use of the conjugate as a vehicle with which to deliver a therapeutic peptide in a controlled manner. Polymeric drug delivery systems are known in the art. See, for example, Dunn et al., Eds. POLYMERIC DRUGS AND DRUG DELIVERY SYSTEMS, ACS Symposium Series Vol. 469, American Chemical Society, Washington, D.C. 1991. Those of skill in the art will appreciate that substantially any known drug delivery system is applicable to the conjugates of the present invention.

Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in the glyco-conjugates of the invention include, but are not limited to, alkyl celluloses, hydroxyalkyl celluloses, cellulose ethers, cellulose esters, and nitrocelluloses. Particularly preferred members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

These and the other polymers discussed herein can be readily obtained from commercial sources such as Sigma Chemical Co. (St. Louis, Mo.), Polysciences (Warrenton, Pa.), Aldrich (Milwaukee, Wis.), Fluka (Ronkonkoma, N.Y.), and BioRad (Richmond, Calif.), or else synthesized from monomers obtained from these suppliers using standard techniques.

Representative biodegradable polymers of use in the conjugates of the invention include, but are not limited to, polylactides, polyglycolides and copolymers thereof, poly(ethylene terephthalate), poly(butyric acid), poly(valeric acid), poly(lactide-co-caprolactone), poly(lactide-co-glycolide), polyanhydrides, polyorthoesters, blends and copolymers thereof. Of particular use are compositions that form gels, such as those including collagen, pluronics and the like.

The polymers of use in the invention include “hybrid’ polymers that include water-insoluble materials having within at least a portion of their structure, a bioresorbable molecule. An example of such a polymer is one that includes a water-insoluble copolymer, which has a bioresorbable region, a hydrophilic region and a plurality of crosslinkable functional groups per polymer chain.

For purposes of the present invention, “water-insoluble materials” includes materials that are substantially insoluble in water or water-containing environments. Thus, although certain regions or segments of the copolymer may be hydrophilic or even water-soluble, the polymer molecule, as a whole, is not substantially soluble in water.

For purposes of the present invention, the term “bioresorbable molecule” includes a region that is capable of being metabolized or broken down and resorbed and/or eliminated through normal excretory routes by the body. Such metabolites or break down products are preferably substantially non-toxic to the body.

The bioresorbable region may be either hydrophobic or hydrophilic, so long as the copolymer composition as a whole is not rendered water-soluble. Thus, the bioresorbable region is selected based on the preference that the polymer, as a whole, remains water-insoluble. Accordingly, the relative properties, i.e., the kinds of functional groups contained by, and the relative proportions of the bioresorbable region, and the hydrophilic region are selected to ensure that useful bioresorbable compositions remain water-insoluble.

Exemplary resorbable polymers include, for example, synthetically produced resorbable block copolymers of poly(α-hydroxy-carboxylic acid)/poly(oxyalkylene, (see, Cohn et al., U.S. Pat. No. 4,826,945). These copolymers are not crosslinked and are water-soluble so that the body can excrete the degraded block copolymer compositions. See, Younes et al., J Biomed. Mater. Res. 21: 1301-1316 (1987); and Cohn et al., J Biomed. Mater. Res. 22: 993-1009 (1988).

Presently preferred bioresorbable polymers include one or more components selected from poly(esters), poly(hydroxy acids), poly(lactones), poly(amides), poly(ester-amides), poly(amino acids), poly(anhydrides), poly(orthoesters), poly(carbonates), poly(phosphazines), poly(phosphoesters), poly(thioesters), polysaccharides and mixtures thereof. More preferably still, the biosresorbable polymer includes a poly(hydroxy) acid component. Of the poly(hydroxy) acids, polylactic acid, polyglycolic acid, polycaproic acid, polybutyric acid, polyvaleric acid and copolymers and mixtures thereof are preferred.

In addition to forming fragments that are absorbed in vivo (“bioresorbed”), preferred polymeric coatings for use in the methods of the invention can also form an excretable and/or metabolizable fragment.

Higher order copolymers can also be used in the present invention. For example, Casey et al., U.S. Pat. No. 4,438,253, which issued on Mar. 20, 1984, discloses tri-block copolymers produced from the transesterification of poly(glycolic acid) and an hydroxyl-ended poly(alkylene glycol). Such compositions are disclosed for use as resorbable monofilament sutures. The flexibility of such compositions is controlled by the incorporation of an aromatic orthocarbonate, such as tetra-p-tolyl orthocarbonate into the copolymer structure.

Other polymers based on lactic and/or glycolic acids can also be utilized. For example, Spinu, U.S. Pat. No. 5,202,413, which issued on Apr. 13, 1993, discloses biodegradable multi-block copolymers having sequentially ordered blocks of polylactide and/or polyglycolide produced by ring-opening polymerization of lactide and/or glycolide onto either an oligomeric diol or a diamine residue followed by chain extension with a di-functional compound, such as, a diisocyanate, diacylchloride or dichlorosilane.

Bioresorbable regions of coatings useful in the present invention can be designed to be hydrolytically and/or enzymatically cleavable. For purposes of the present invention, “hydrolytically cleavable” refers to the susceptibility of the copolymer, especially the bioresorbable region, to hydrolysis in water or a water-containing environment. Similarly, “enzymatically cleavable” as used herein refers to the susceptibility of the copolymer, especially the bioresorbable region, to cleavage by endogenous or exogenous enzymes.

When placed within the body, the hydrophilic region can be processed into excretable and/or metabolizable fragments. Thus, the hydrophilic region can include, for example, polyethers, polyalkylene oxides, polyols, poly(vinyl pyrrolidine), poly(vinyl alcohol), poly(alkyl oxazolines), polysaccharides, carbohydrates, peptides, proteins and copolymers and mixtures thereof. Furthermore, the hydrophilic region can also be, for example, a poly(alkylene) oxide. Such poly(alkylene) oxides can include, for example, poly(ethylene) oxide, poly(propylene) oxide and mixtures and copolymers thereof.

Polymers that are components of hydrogels are also useful in the present invention. Hydrogels are polymeric materials that are capable of absorbing relatively large quantities of water. Examples of hydrogel forming compounds include, but are not limited to, polyacrylic acids, sodium carboxymethylcellulose, polyvinyl alcohol, polyvinyl pyrrolidine, gelatin, carrageenan and other polysaccharides, hydroxyethylenemethacrylic acid (HEMA), as well as derivatives thereof, and the like. Hydrogels can be produced that are stable, biodegradable and bioresorbable. Moreover, hydrogel compositions can include subunits that exhibit one or more of these properties.

Bio-compatible hydrogel compositions whose integrity can be controlled through crosslinking are known and are presently preferred for use in the methods of the invention. For example, Hubbell et al., U.S. Pat. Nos. 5,410,016, which issued on Apr. 25, 1995 and 5,529,914, which issued on Jun. 25, 1996, disclose water-soluble systems, which are crosslinked block copolymers having a water-soluble central block segment sandwiched between two hydrolytically labile extensions. Such copolymers are further end-capped with photopolymerizable acrylate functionalities. When crosslinked, these systems become hydrogels. The water soluble central block of such copolymers can include poly(ethylene glycol); whereas, the hydrolytically labile extensions can be a poly(α-hydroxy acid), such as polyglycolic acid or polylactic acid. See, Sawhney et al., Macromolecules 26: 581-587 (1993).

In another preferred embodiment, the gel is a thermoreversible gel. Thermoreversible gels including components, such as pluronics, collagen, gelatin, hyalouronic acid, polysaccharides, polyurethane hydrogel, polyurethane-urea hydrogel and combinations thereof are presently preferred.

In yet another exemplary embodiment, the conjugate of the invention includes a component of a liposome. Liposomes can be prepared according to methods known to those skilled in the art, for example, as described in Eppstein et al., U.S. Pat. No. 4,522,811, which issued on Jun. 11, 1985. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its pharmaceutically acceptable salt is then introduced into the container. The container is then swirled by hand to free lipid material from the sides of the container and to disperse lipid aggregates, thereby forming the liposomal suspension.

The above-recited microparticles and methods of preparing the microparticles are offered by way of example and they are not intended to define the scope of microparticles of use in the present invention. It will be apparent to those of skill in the art that an array of microparticles, fabricated by different methods, are of use in the present invention.

### Methods

In addition to the compositions discussed above, the present invention provides methods for preparing glyco-conjugates. Moreover, the invention provides methods of preventing, curing or ameliorating a disease state by administering a conjugate of the invention to a subject at risk of developing the disease or a subject that has the disease.

Thus, the invention provides a method of forming a glyco-conjugate between a modifying group and a glycosyl-containing compound, e.g., a glycopeptide, or a glycolipid. For clarity of illustration, the invention is illustrated with reference to a conjugate formed between a glycopeptide and an activated modifying group that includes a water-soluble polymer. Those of skill will appreciate that the invention equally encompasses methods of forming conjugates of glycolipids with water-soluble polymers, and forming conjugates between glycopeptides and glycolipids and modifying groups other than water-soluble polymers.

In a representative embodiment, the method includes: (a) contacting a peptide comprising a glycosyl residue with;

(i) an acylating agent comprising an activated acyl moiety that is reactive with an N-, O- or S-containing residue on the peptide; and

(ii) an enzyme for which said acylating agent is a substrate, under conditions appropriate to acylate said glycosyl residue.

In exemplary embodiments, the conjugate is formed between a water-soluble polymer, a therapeutic moiety, targeting moiety or a biomolecule, and a glycosylated peptide. The polymer, therapeutic moiety or biomolecule is conjugated to the peptide via a glycosyl linking group, which is interposed between, and covalently linked to, both the peptide (directly or through an intervening glycosyl linker) and the modifying group (e.g., water-soluble polymer). The method includes contacting the glycopeptide with an activated modifying group and an enzyme for which the activated modifying group is a substrate. The components of the reaction mixture are combined under conditions appropriate to acylate a selected glycosyl residue on the glycopeptide, thereby preparing the conjugate. In an exemplary embodiment, the glycosyl residue is acylated by the acylating agent at a site that is a member selected from an OH, NH₂ and SH.

The acceptor peptide is typically synthesized de novo, or recombinantly expressed in a prokaryotic cell (e.g., bacterial cell, such as E. coli) or in a eukaryotic cell such as a mammalian, yeast, insect, fungal or plant cell. The peptide can be either a full-length protein or a fragment. Moreover, the peptide can be a wild type or mutated peptide. In an exemplary embodiment, the peptide includes a mutation that adds one or more N- or O-linked glycosylation sites to the peptide sequence.

The method of the invention also provides for modification of incompletely glycosylated peptides that are produced recombinantly. Many recombinantly produced glycoproteins are incompletely glycosylated, exposing carbohydrate residues that may have undesirable properties, e.g., immunogenicity, recognition by the RES. The incomplete glycosyl residue can be masked using a water-soluble polymer.

Those of skill will appreciate that the invention can be practiced using substantially any peptide or glycopeptide from any source. Exemplary peptides with which the invention can be practiced are set forth in WO 03/031464, and the references set forth therein.

Peptides modified by the methods of the invention can be synthetic or wild-type peptides or they can be mutated peptides, produced by methods known in the art, such as site-directed mutagenesis. Glycosylation of peptides is typically either N-linked or O-linked. An exemplary N-linkage is the attachment of the modified sugar to the side chain of an asparagine residue. The tripeptide sequences asparagine-X-serine and asparagine-X-threonine, where X is any amino acid except proline, are the recognition sequences for enzymatic attachment of a carbohydrate moiety to the asparagine side chain. Thus, the presence of either of these tripeptide sequences in a polypeptide creates a potential glycosylation site. O-linked glycosylation refers to the attachment of one sugar (e.g., N-acetylgalactosamine, galactose, mannose, GlcNAc, glucose, fucose or xylose) to the hydroxy side chain of a hydroxyamino acid, preferably serine or threonine, although unusual or non-natural amino acids, e.g., 5-hydroxyproline or 5-hydroxylysine may also be used.

Moreover, in addition to peptides, the methods of the present invention can be practiced with other biological structures (e.g., glycolipids, lipids, sphingoids, ceramides, whole cells, and the like, containing a glycosylation site).

Addition of glycosylation sites to a peptide or other structure is conveniently accomplished by altering the amino acid sequence such that it contains one or more glycosylation sites. The addition may also be made by the incorporation of one or more species presenting an —OH group, preferably serine or threonine residues, within the sequence of the peptide (for O-linked glycosylation sites). The addition may be made by mutation or by full chemical synthesis of the peptide. The peptide amino acid sequence is preferably altered through changes at the DNA level, particularly by mutating the DNA encoding the peptide at preselected bases such that codons are generated that will translate into the desired amino acids. The DNA mutation(s) are preferably made using methods known in the art.

In an exemplary embodiment, the glycosylation site is added by shuffling polynucleotides. Polynucleotides encoding a candidate peptide can be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer, Proc. Natl. Acad. Sci. USA 91:10747-10751 (1994); Stemmer, Nature 370:389-391 (1994); and U.S. Pat. Nos. 5,605,793, 5,837,458, 5,830,721 and 5,811,238.

The present invention also provides means of adding (or removing) one or more selected glycosyl residues to a peptide, after which a modified sugar is conjugated to at least one of the selected glycosyl residues of the peptide. The present embodiment is useful, for example, when it is desired to conjugate the modified sugar to a selected glycosyl residue that is either not present on a peptide or is not present in a desired amount. Thus, prior to coupling a modified sugar to a peptide, the selected glycosyl residue is conjugated to the peptide by enzymatic or chemical coupling. In another embodiment, the glycosylation pattern of a glycopeptide is altered prior to the conjugation of the modified sugar by the removal of a carbohydrate residue from the glycopeptide. See, for example WO 98/31826.

Addition or removal of any carbohydrate moieties present on the glycopeptide is accomplished either chemically or enzymatically. Chemical deglycosylation is preferably brought about by exposure of the polypeptide variant to the compound trifluoromethanesulfonic acid, or an equivalent compound. This treatment results in the cleavage of most or all sugars except the linking sugar (N-acetylglucosamine or N-acetylgalactosamine), while leaving the peptide intact. Chemical deglycosylation is described by Hakimuddin et al., Arch. Biochem. Biophys. 259: 52 (1987) and by Edge et al., Anal. Biochem. 118: 131 (1981). Enzymatic cleavage of carbohydrate moieties on polypeptide variants can be achieved by the use of a variety of endo- and exo-glycosidases as described by Thotakura et al., Meth. Enzymol. 138: 350 (1987).

Chemical addition of glycosyl moieties is carried out by any art-recognized method. Enzymatic addition of sugar moieties is preferably achieved using a modification of the methods set forth herein, substituting native glycosyl units for the modified sugars used in the invention. Other methods of adding sugar moieties are disclosed in U.S. Pat. Nos. 5,876,980, 6,030,815, 5,728,554, and 5,922,577.

Exemplary attachment points for selected glycosyl residue include, but are not limited to: (a) consensus sites for N-linked glycosylation, and sites for O-linked glycosylation; (b) terminal glycosyl moieties that are acceptors for a glycosyltransferase; (c) arginine, asparagine and histidine; (d) free carboxyl groups; (e) free sulfhydryl groups such as those of cysteine; (f) free hydroxyl groups such as those of serine, threonine, or hydroxyproline; (g) aromatic residues such as those of phenylalanine, tyrosine, or tryptophan; or (h) the amide group of glutamine. Exemplary methods of use in the present invention are described in WO 87/05330 published Sep. 11, 1987, and in Aplin and Wriston, CRC CRIT. REV. BIOCHEM., pp. 259-306 (1981).

In one embodiment, the invention provides a method for linking two or more peptides through a linking group. The linking group is of any useful structure and may be selected from straight- and branched-chain structures. Preferably, each terminus of the linker, which is attached to a peptide, includes a modified sugar.

In an exemplary method of the invention, two peptides are linked together via a linker moiety that includes a polymeric (e.g., PEG linker). The focus on a PEG linker that includes two glycosyl groups is for purposes of clarity and should not be interpreted as limiting the identity of linker arms of use in this embodiment of the invention.

Exemplary peptides with which the present invention can be practiced, methods of adding or removing glycosylation sites, and adding or removing glycosyl structures or substructures are described in detail in WO03/031464 and related U.S. and PCT applications.

In addition to the compositions discussed above, the present invention provides methods for preparing peptide-conjugates comprising a lipid-based linker and a modifying group. Moreover, the invention provides methods of preventing, curing or ameliorating a disease state by administering a conjugate of the invention to a subject at risk of developing the disease or to a subject who has the disease.

Thus, the invention provides a method of forming a peptide-conjugate between a modifying group and a lipid-containing compound, e.g., a lipopeptide. For clarity of illustration, the invention is illustrated with reference to a conjugate formed between a peptide and an activated modifying group comprising a lipid and a water-soluble polymer. Those of skill will appreciate that the invention equally encompasses methods of forming conjugates between peptides and modifying groups other than water-soluble polymers.

In exemplary embodiments, the conjugate is formed between a water-soluble polymer, a therapeutic moiety, targeting moiety or a biomolecule, and a peptide. The polymer, therapeutic moiety or biomolecule is conjugated to the peptide via a lipid linking group, which is interposed between, and covalently linked to, both the peptide and the modifying group (e.g., water-soluble polymer). The method includes contacting the peptide with an activated modifying group comprising a lipid linker and a water-soluble polymer, and an enzyme for which the activated modifying group is a substrate. The components of the reaction mixture are combined under conditions appropriate to link the amino acid residue on the peptide, to the activated lipid linker comprising the modifying group thereby preparing the conjugate.

In one embodiment, the lipid linker is a fatty acid derivative comprising repeating methylene units. In this embodiment, the fatty acid may be linked to the peptide by a thioester bond with cysteine (i.e. thio-palmitoylation) or in amide linkage to an N-terminal glycine (N-acylation; Knoll et al. Methods in Enzymol. 250:405 (1995)) or an c-amine of an internal lysine (Hackett M. et al. Science 266:433-435 (1994)).

In another embodiment, the lipid linker is a fatty acid comprising repeating isoprene units. In this embodiment, the peptide conjugate may comprise one or more modified lipids linked through one or more thioester linkages with cysteine residues of the peptide. In one aspect, modified lipids for use in the invention may be prepared according to one or more of the methods outlined in Scheme 1-3 below.

Scheme 1 sets forth an exemplary route to PEGylated isoprenyl compounds of use in the present invention. Starting compound 1 is produced by protecting a commercially available alcohol (e.g., farnesol, geraniol). The selection of an appropriate protecting agent is within the ability of those of skill in the art.

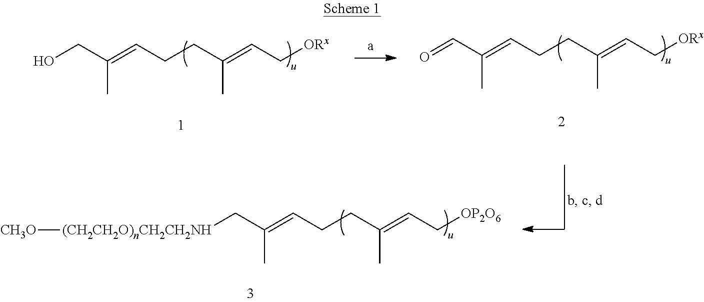

The protected alcohol is then selectively oxidized to compound 1 using an art-recognized method. See, e.g., Bukhtiyarov et al., J. Biol. Chem., 270: 19035-19040 (1995). For example, the alcohol can be formed by the action of t-butyl hydroperoxide and H₂SeO₃.

In step a, the unprotected hydroxyl moiety is selectively oxidized to the corresponding aldehyde. Exemplary oxidation conditions include catalytic oxidation using a supported platinum group metal ion, e.g., Ru—Al—Mg Hydrotalcite, Ru—Al—Co hydrotalcite, Pd(II) hydrotalcite, Pd Cluster Complex/TiO₂ and the like. The resulting carbonyl compound, e.g., aldehyde, is reductively aminated with m-PEG-amine (b), and the protecting group is removed (c). The exposed hydroxyl moiety is converted to the corresponding diphosphate (d). See, Holloway et al., Biochem. J., 104: 57-70 (1967). Exemplary phosphorylation conditions for converting the hydroxyl to the diphosphate are bis-(triethylammonium)hydrogen phosphate in the presence of a large excess of CCl₃CN in acetonitrile (Bukhtiyarov et al., supra).

Scheme 2 sets forth a route to compounds of use in a method of the invention in which the m-PEG moiety is tethered to the isoprenyl moiety through an ether linkage. Thus, alcohol 1 is reacted with an activated m-PEG species, e.g., a halo or sulfonate derivative under conditions appropriate to form the ether (e). The protecting group is removed (c) and the resulting alcohol is phosphorylated as discussed above.

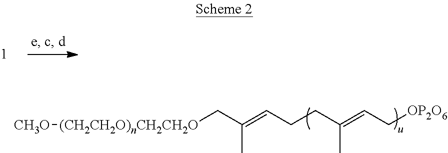

Alternatively, a reactive starting material can be assembles using other recognized methods. See, for example, Mehta et al., The Chemistry of Dienes and Polyenes, Wiley Interscience, NY, 1997.

In another embodiment, a linker is interposed between the m-PEG moiety and the isoprenyl moiety. An exemplary linker is based upon a amino carboxylic acid. Thus, according to Scheme 3, aldehyde 2 is reductively aminated with an amino carboxylic acid (f). The acid is activated, e.g., active ester, acid halide, and coupled with m-PEG amine, forming the corresponding amide (g). The protecting group on the hydroxyl of the amide is removed (c) and the hydroxyl moiety is phosphorylated.

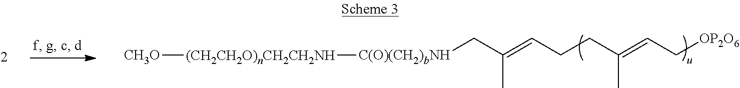

In another embodiment, the lipid linker is a fatty acid comprising repeating methylene units. In this embodiment, the peptide conjugate may comprise one or more modified lipids linked through one or more amide linkages, e.g., on the α-amino group of an N-terminal glycine, or the E-amino group of an internal lysine. In a related embodiment, the peptide conjugate may comprise one or more modified lipids comprised of repeating methylene units, and these lipids may be linked through one or more thioester linkages with cysteine residues of the peptide. In a further related embodiment, the peptide conjugate may comprise one or more modified lipids comprised of repeating methylene units, and the modified lipids may be independently linked through both amide and thioester linkages on the same peptide. In one aspect, modified lipids for use in these embodiments of the invention may be prepared according to one or more of the methods outlined in Scheme 4.

Derivatives of palmitic acid can be activated for use with a transferase by converting the carboxylic group to a thioester. In an exemplary embodiment, set forth in scheme 4, the thioester is a CoA thioester. In Scheme 4, 16-OH palmitic acid is reacted with an activated poly(ethylene glycol) species under conditions appropriate for the formation of the corresponding ether. The carboxylic acid of the resulting PEG-palmitic acid ether is activated by conversion to an activated ester (e.g., NHS), an anhydride or the like. The activated species is converted to the corresponding Coenzyme A thioester by combining the activated species and Coenzyme A under conditions appropriate for the coupling to occur. The formation of CoA thioesters by this route and other analogous routes is known in the art. See, for example, Kutner et al., Proc. Natl. Acad. Sci. U.S.A. 83: 6781-4 (1986).

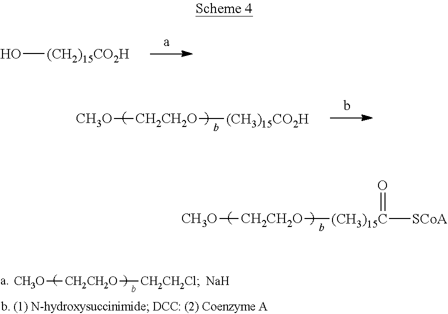

The acceptor peptide is typically synthesized de novo, or recombinantly expressed in a prokaryotic cell (e.g., bacterial cell, such as E. coli) or in a eukaryotic cell such as a mammalian, yeast, insect, fungal or plant cell. The peptide can be either a full-length protein or a fragment. Moreover, the peptide can be a wild type or mutated peptide.

Exemplary peptides that can be modified using the methods of the invention are set forth in Table 1.

TABLE 1

Hormones and Growth Factors
G-CSF
GM-CSF
M-CSF
TPO
EPO
EPO variants
alpha-TNF
Leptin
Enzymes and Inhibitors
t-PA
t-PA variants
Urokinase
Factors VII, VIII, IX, X
Dnase
Glucocerebrosidase
Hirudin
α1 antitrypsin
Antithrombin III
Cytokines and Chimeric Cytokines
Interleukin-1 (IL-1), 1B, 2, 3, 4
Interferon-alpha (IFN-alpha)
IFN-alpha-2b
IFN-beta
IFN-gamma
Chimeric diptheria toxin-IL-2
Receptors and Chimeric Receptors
CD4
Tumor Necrosis Factor (TNF) receptor
Alpha-CD20
MAb-CD20
MAb-alpha-CD3
MAb-TNF receptor
MAb-CD4
PSGL-1
MAb-PSGL-1
Complement
GlyCAM or its chimera
N-CAM or its chimera
Monoclonal Antibodies
(Immunoglobulins)
MAb-anti-RSV
MAb-anti-IL-2 receptor
MAb-anti-CEA
MAb-anti-platelet IIb/IIIa receptor
MAb-anti-EGF
MAb-anti-Her-2 receptor
Cells
Red blood cells
White blood cells (e.g., T cells, B cells,
dendritic cells, macrophages, NK cells,
neutrophils, monocytes and the like
Stem cells

Other exemplary peptides that are modified by the methods of the invention include members of the immunoglobulin family (e.g., antibodies, MI-IC molecules, T cell receptors, and the like), intercellular receptors (e.g., integrins, receptors for hormones or growth factors and the like) lectins, and cytokines (e.g., interleukins). Additional examples include tissue-type plasminogen activator (t-PA), renin, clotting factors such as factors V-XII, bombesin, thrombin, hematopoietic growth factor, colony stimulating factors, viral antigens, complement proteins, α1-antitrypsin, erythropoietin, P-selectin glycopeptide ligand-1 (PSGL-1), granulocyte-macrophage colony stimulating factor, anti-thrombin III, interleukins, interferons, proteins A and C, fibrinogen, herceptin, leptin, glycosidases, HS-glycoprotein, serum proteins (e.g., α-acid glycoprotein, fetuin, α-fetal protein), β2-glycoprotein, NeuroTropin III (NT III), Bone Morphogenic Peptide (BMP), BMP-II, Fibroblast Growth Factor (FGF), FGF-20, glutaminase-interacting protein (GIP), among many others. This list of polypeptides is exemplary, not exclusive. The methods are also useful for modifying fusion and chimeric proteins, including, but not limited to, chimeric proteins that include a moiety derived from an immunoglobulin, such as IgG, or a fragment of an immunoglobin, e.g., FAb (Fc domain). The exemplary peptides provided herein are intended to provide a selection of the peptides with which the present invention can be practiced; as such, they are non-limiting. Those of skill will appreciate that the invention can be practiced using substantially any peptide from any source.

Peptides modified by the methods of the invention can be synthetic or wild-type peptides or they can be mutated peptides, produced by methods known in the art, such as site-directed mutagenesis.

### Enzyme Classes

Aspects of the present invention make use of enzymes that form a bond between an activated acyl moiety and a heteroatom found on a sugar nucleus. The enzymes useful in practicing the present invention include, but are not limited to, wild-type and mutant proteases, lipases, esterases, acylases, acyltransferases, glycosyltransferases, sufotransferases, glycosidases, and the like. An exemplary mutant is one in which one or more amino acid residues in the active site are altered to provide an enzyme with synthetic activity that is improved relative to the activity in the corresponding wild-type enzyme. In an exemplary embodiment, the enzyme is a member selected from a lipase, a protease, an esterase, an acylase and an acyltransferase. In another exemplary embodiment, the enzyme has an amino acid sequence that is a wild-type sequence for said enzyme. In another exemplary embodiment, the enzyme is a mutated enzyme which has a mutated amino acid sequence. In another exemplary embodiment, the mutated enzyme has an acylation activity that is enhanced relative to a corresponding wild-type enzyme. In another exemplary embodiment, the mutated amino acid sequence comprises a mutation wherein an amino acid residue implicated in hydrolysis of a member selected from an amide and an ester, is replaced by an amino acid residue that is not implicated in the hydrolysis.

### Acyl Transfer

The discovery that some enzymes are catalytically active in organic solvents has greatly expanded their use as biocatalysts. In this medium these enzymes show a new catalytic behavior. For example lipases catalyse esterification and transesterification reactions in organic media. These properties enable the production of compounds which are difficult to obtain using chemical methods.

### Proteases

A protease is employed in some embodiments of the invention. Proteases are known in the art to catalyze the attachment of amino acids to sugars through esterification. (Davis, (WO 03/014371, published Feb. 20, 2003). In this publication, a vinyl ester amino acid group was reacted with a carbohydrate acyl acceptor in the presence of the serine protease subtilisin derived from Bacillus lentus. Wild-type proteases can be additionally be isolated from Bacillus amyloliquefaciens. Mutant proteases can be made according to the teachings of, for example, PCT Publication Nos. WO 95/10615 and WO 91/06637, which are hereby incorporated by reference. Other proteases of use in this invention include serine proteases (such as chymotrypsin, plasmin, and thrombin), cysteine proteases (such as cathepsin B and papain), and aspartic endopeptidases (such as pepsin A, chymosin, cathepsin D).

In an exemplary embodiment, utilizing a protease, the link between the sugar moiety and the modifying group is an amino acid that is derivatized with the modifying group. The sugar and amino acid are linked through an amide moiety formed by the protease.

### Lipases

A lipase is used in some embodiments of the invention. The use of lipases in the acylation of saccharides has been previously reported. For example, regioselective acylations of alkyl β-D-xylopyranosides using lipase PS in organic solvents was reported by Lopez. (Lopez et al., J. Org. Chem., 59, 7027-7032 (1994). Another group also utilized lipase PS in order to catalyze the transfer of acetyl groups onto sialic acids in vinyl acetate. (Lo et al., Bioorg. Med. Chem. Lett., 9, 709-712 (1999)). Regioselective disaccharide acylation in tert-butyl alcohol catalyzed by Candida antarctica lipase has also been reported. (Woudenberg van-Oosterom et al., Biotechnol. Bioeng., 49, 328-333 (1996)). Immobilized versions of the Candida antarctica lipase have also been used to acylate hydroxypropyl cellulose in tert-butanol. (Sereti et al., Biotechnol Bioeng., 72(4), 495-500 (2001)). Other lipases of use in this invention include lipoprotein lipase, triacylglycerol lipase, diglyceride lipase, and postheparin lipase.

### Esterases

Esterases can also be used in some embodiments of the invention. Acetylation of cellobiose and cellulose was shown to be catalyzed in aqueous medium in the presence of isopropenyl acetate by an intracellular carboxylesterase from Arthrobacter viscosus. (Cui et al., Enzyme Microb. Technol., 24, 200-208 (1999)). Another group acetylated the amino groups of chitobiose and chitotetraose in an aqueous solution of 3M sodium acetate using a chitin deacetylase from Colletotrichum lindemuthianum (Tokuyasu et al., Carbohydr. Res., 322, 26-31 (1999)). A third group utilized acetylxylan esterase (AcXE) from Schizophyllum commune to catalyze acetyl group transfer to methyl β-D-xylopyranoside, methyl β-D-cellobioside, methyl β-D-glucopyranoside, cellotetraose, 2-deoxy-D-glucose, D-mannose, β-1,4-mannobiose, β-1,4-mannopentaose, β-1,4-mannohexaose, β-1,4-xylobiose, and β-1,4-xylopentaose. (Biely et al., Biochimica et Biophysica Acta, 1623, 62-71 (2003)). Acetylation of secondary alcohols was also achieved by transesterification from vinyl acetate by a feruloyl esterase from Humicola insolens. (Hatzakis et al., J. Mol. Catal., B Enzym. 21, 309-311 (2003). Other esterases of use in this invention include choline esterase, sterol esterase, hydroxycinnamoyl esterase, acetylsalicyclic acid esterase, and polyneuridine esterase.

### Acylases

Acylases can also be used in some embodiments of the invention. Exemplary acylases of use in this invention include aminoacylase I, L-amino-acid acylase, penicillin acylase, acetyl-CoA acylase, acyl-lysine deacylase, aculeacin A acylase, succinyl-CoA acylase, and acetyl-aspartic deaminase.

### Acetyltransferases

In another embodiment of the invention, acyl transfer is accomplished by an acetyltransferase. The use of acetyltransferases in the acylation of saccharides has been previously reported. O-acetylation at the 9 position of sialic acid has been shown to occur from the product of several genes in the COS cell system (Shi et al., Glycobiology, 8(2), 199-205 (1998)). Maltose O-acetyltransferase (MAT) from Escherichia coli is known to catalyze acetyl group transfer to the C6 positions of glucose and maltose. (Leggio et al., Biochemistry, 42, 5225-5235 (2003)). This same group also utilized galactoside acetyltransferase (GAT) to catalyze acetyl group transfer to galactosyl units. Other acetyltransferases of use in this invention include spermidine acetyltransferase, diamine N-acetyltransferase, and sialate O-acetyltransferase.

### Sugar Transfer

In addition to the enzymes discussed above in the context of forming the acyl-linked conjugate, the glycosylation pattern of the conjugate and the starting substrates (e.g., peptides, lipids) can be elaborated, trimmed back or otherwise modified by methods utilizing other enzymes. For example, in one embodiment, the glycosyl acceptor for the acyl moiety is conjugated to the peptide (or aglycone) or to a glycosyl residue on the peptide (or aglycone) using an enzymatically-mediated sugar transfer reaction. The methods of remodeling peptides and lipids using enzymes that transfer a sugar donor to an acceptor are discussed in great detail in DeFrees, WO 03/031464 A2, published Apr. 17, 2003. A brief summary of selected enzymes of use in the present method is set forth below.

### Glycosyltransferases

Glycosyltransferases catalyze the addition of activated sugars (donor NDP-sugars), in a step-wise fashion, to a protein, glycopeptide, lipid or glycolipid or to the non-reducing end of a growing oligosaccharide. N-linked glycopeptides are synthesized via a transferase and a lipid-linked oligosaccharide donor Dol-PP-NAG₂Glc₃Man₉ in an en block transfer followed by trimming of the core. In this case the nature of the “core” saccharide is somewhat different from subsequent attachments. A very large number of glycosyltransferases are known in the art.

The glycosyltransferase to be used in the present invention may be any as long as it can utilize the modified sugar as a sugar donor. Examples of such enzymes include Leloir pathway glycosyltransferase, such as galactosyltransferase, N-acetylglucosaminyltransferase, N-acetylgalactosaminyltransferase, fucosyltransferase, sialyltransferase, mannosyltransferase, xylosyltransferase, glucurononyltransferase and the like.

For enzymatic saccharide syntheses that involve glycosyltransferase reactions, glycosyltransferase can be cloned, or isolated from any source. Many cloned glycosyltransferases are known, as are their polynucleotide sequences. See, e.g., “The WWW Guide To Cloned Glycosyltransferases,” Taniguchi et al., 2002, Handbook of Glycosyltransferases and Related Genes, Springer, Tokyo. Glycosyltransferase amino acid sequences and nucleotide sequences encoding glycosyltransferases from which the amino acid sequences can be deduced are also found in various publicly available databases, including GenBank, Swiss-Prot, EMBL, and others.

Glycosyltransferases that can be employed in the methods of the invention include, but are not limited to, galactosyltransferases, fucosyltransferases, glucosyltransferases, N-acetylgalactosaminyltransferases, N-acetylglucosaminyltransferases, glucuronyltransferases, sialyltransferases, mannosyltransferases, glucuronic acid transferases, galacturonic acid transferases, and oligoglycosyltransferases. Suitable glycosyltransferases include those obtained from eukaryotes, as well as from prokaryotes.

DNA encoding glycosyltransferases may be obtained by chemical synthesis, by screening reverse transcripts of mRNA from appropriate cells or cell line cultures, by screening genomic libraries from appropriate cells, or by combinations of these procedures. Screening of mRNA or genomic DNA may be carried out with oligonucleotide probes generated from the glycosyltransferases gene sequence. Probes may be labeled with a detectable group such as a fluorescent group, a radioactive atom or a chemiluminescent group in accordance with known procedures and used in conventional hybridization assays. In the alternative, glycosyltransferases gene sequences may be obtained by use of the polymerase chain reaction (PCR) procedure, with the PCR oligonucleotide primers being produced from the glycosyltransferases gene sequence. See, U.S. Pat. No. 4,683,195 to Mullis et al. and U.S. Pat. No. 4,683,202 to Mullis.

The glycosyltransferase may be synthesized in host cells transformed with vectors containing DNA encoding the glycosyltransferases enzyme. Vectors are used either to amplify DNA encoding the glycosyltransferases enzyme and/or to express DNA which encodes the glycosyltransferases enzyme. An expression vector is a replicable DNA construct in which a DNA sequence encoding the glycosyltransferases enzyme is operably linked to suitable control sequences capable of effecting the expression of the glycosyltransferases enzyme in a suitable host. The need for such control sequences will vary depending upon the host selected and the transformation method chosen. Generally, control sequences include a transcriptional promoter, an optional operator sequence to control transcription, a sequence encoding suitable mRNA ribosomal binding sites, and sequences which control the termination of transcription and translation. Amplification vectors do not require expression control domains. All that is needed is the ability to replicate in a host, usually conferred by an origin of replication, and a selection gene to facilitate recognition of transformants.

In an exemplary embodiment, the invention utilizes a prokaryotic enzyme. Such glycosyltransferases include enzymes involved in synthesis of lipooligosaccharides (LOS), which are produced by many gram negative bacteria (Preston et al., Critical Reviews in Microbiology 23(3): 139-180 (1996)). Such enzymes include, but are not limited to, the proteins of the rfa operons of species such as E. coli and Salmonella typhimurium, which include a β1,6 galactosyltransferase and a β1,3 galactosyltransferase (see, e.g., EMBL Accession Nos. M80599 and M86935 (E. coli); EMBL Accession No. S56361 (S. typhimurium)), a glucosyltransferase (Swiss-Prot Accession No. P25740 (E. coli), an β1,2-glucosyltransferase (rfaJ)(Swiss-Prot Accession No. P27129 (E. coli) and Swiss-Prot Accession No. P19817 (S. typhimurium)), and an β1,2-N-acetylglucosaminyltransferase (rfaK)(EMBL Accession No. U00039 (E. coli). Other glycosyltransferases for which amino acid sequences are known include those that are encoded by operons such as rfaB, which have been characterized in organisms such as Klebsiella pneumoniae, E. coli, Salmonella typhimurium, Salmonella enterica, Yersinia enterocolitica, Mycobacterium leprosum, and the rhl operon of Pseudomonas aeruginosa.

Also suitable for use in the present invention are glycosyltransferases that are involved in producing structures containing lacto-N-neotetraose, D-galactosyl-β-1,4-N-acetyl-D-glucosaminyl-β-1,3-D-galactosyl-β-1,4-D-glucose, and the P^{k} blood group trisaccharide sequence, D-galactosyl-α-1,4-D-galactosyl-β-1,4-D-glucose, which have been identified in the LOS of the mucosal pathogens Neisseria gonnorhoeae and N. meningitidis (Scholten et al., J. Med. Microbiol. 41: 236-243 (1994)). The genes from N. meningitidis and N. gonorrhoeae that encode the glycosyltransferases involved in the biosynthesis of these structures have been identified from N. meningitidis immunotypes L3 and L1 (Jennings et al., Mol. Microbiol. 18: 729-740 (1995)) and the N. gonorrhoeae mutant F62 (Gotshlich, J. Exp. Med. 180: 2181-2190 (1994)). In N. meningitidis, a locus consisting of three genes, lgtA, lgtB and lg E, encodes the glycosyltransferase enzymes required for addition of the last three of the sugars in the lacto-N-neotetraose chain (Wakarchuk et al., J. Biol. Chem. 271: 19166-73 (1996)). Recently the enzymatic activity of the lgtB and lgtA gene product was demonstrated, providing the first direct evidence for their proposed glycosyltransferase function (Wakarchuk et al., J. Biol. Chem. 271(45): 28271-276 (1996)). In N. gonorrhoeae, there are two additional genes, lgtD which adds β-D-GalNAc to the 3 position of the terminal galactose of the lacto-N-neotetraose structure and lgtC which adds a terminal α-D-Gal to the lactose element of a truncated LOS, thus creating the P^{k} blood group antigen structure (Gotshlich (1994), supra.). In N. meningitidis, a separate immunotype L1 also expresses the P^{k} blood group antigen and has been shown to carry an lgtC gene (Jennings et al., (1995), supra.). Neisseria glycosyltransferases and associated genes are also described in U.S. Pat. No. 5,545,553 (Gotschlich). Genes for α-1,2-fucosyltransferase and α-1,3-fucosyltransferase from Helicobacter pylori has also been characterized (Martin et al., J. Biol. Chem. 272: 21349-21356 (1997)). Also of use in the present invention are the glycosyltransferases of Campylobacter jejuni (see, for example, http://afmb.cnrs-mrs.fr/˜pedro/CAZY/gtf_{—}42.html).

### Fucosyltransferases

In some embodiments, a glycosyltransferase used in the method of the invention is a fucosyltransferase. Fucosyltransferases are known to those of skill in the art. Exemplary fucosyltransferases include enzymes, which transfer L-fucose from GDP-fucose to a hydroxy position of an acceptor sugar. Fucosyltransferases that transfer non-nucleotide sugars to an acceptor are also of use in the present invention.

In some embodiments, the acceptor sugar is, for example, the GlcNAc in a Galβ(1→3,4)GlcNAcβ-group in an oligosaccharide glycoside. Suitable fucosyltransferases for this reaction include the Galβ(1→3,4)GlcNAcβ1-α(1→3,4)fucosyltransferase (FTIII E.C. No. 2.4.1.65), which was first characterized from human milk (see, Palcic, et al., Carbohydrate Res. 190: 1-11 (1989); Prieels, et al., J. Biol. Chem. 256: 10456-10463 (1981); and Nunez, et al., Can. J. Chem. 59: 2086-2095 (1981)) and the Galβ(1→4)GlcNAcβ-αfucosyltransferases (FTIV, FTV, FTVI) which are found in human serum. FTVII (E.C. No. 2.4.1.65), a sialyl α(2→3)Galβ((1→3)GlcNAcβ fucosyltransferase, has also been characterized. A recombinant form of the Galβ(1→3,4) GlcNAcβ-α(1→3,4)fucosyltransferase has also been characterized (see, Dumas, et al., Bioorg. Med. Letters 1: 425-428 (1991) and Kukowska-Latallo, et al., Genes and Development 4: 1288-1303 (1990)). Other exemplary fucosyltransferases include, for example, α1,2 fucosyltransferase (E.C. No. 2.4.1.69). Enzymatic fucosylation can be carried out by the methods described in Mollicone, et al., Eur. J. Biochem. 191: 169-176 (1990) or U.S. Pat. No. 5,374,655. Cells that are used to produce a fucosyltransferase will also include an enzymatic system for synthesizing GDP-fucose.

Galactosyl transferases

In another group of embodiments, the glycosyltransferase is a galactosyltransferase. Exemplary galactosyltransferases include α(1,3) galactosyltransferases (E.C. No. 2.4.1.151, see, e.g., Dabkowski et al., Transplant Proc. 25:2921 (1993) and Yamamoto et al. Nature 345: 229-233 (1990), bovine (GenBank j04989, Joziasse et al., J. Biol. Chem. 264: 14290-14297 (1989)), murine (GenBank m26925; Larsen et al., Proc. Nat'l. Acad. Sci. USA 86: 8227-8231 (1989)), porcine (GenBank L36152; Strahan et al., Immunogenetics 41: 101-105 (1995)). Another suitable α1,3 galactosyltransferase is that which is involved in synthesis of the blood group B antigen (EC 2.4.1.37, Yamamoto et al., J. Biol. Chem. 265: 1146-1151 (1990) (human)). Yet a further exemplary galactosyltransferase is core Gal-T1.

Also suitable for use in the methods of the invention are β(1,4) galactosyltransferases, which include, for example, EC 2.4.1.90 (LacNAc synthetase) and EC 2.4.1.22 (lactose synthetase) (bovine (D'Agostaro et al., Eur. J. Biochem. 183: 211-217 (1989)), human (Masri et al., Biochem. Biophys. Res. Commun. 157: 657-663 (1988)), murine (Nakazawa et al., J. Biochem. 104: 165-168 (1988)), as well as E.C. 2.4.1.38 and the ceramide galactosyltransferase (EC 2.4.1.45, Stahl et al., J. Neurosci. Res. 38: 234-242 (1994)). Other suitable galactosyltransferases include, for example, α1,2 galactosyltransferases (from e.g., Schizosaccharomyces pombe, Chapell et al., Mol. Biol. Cell 5: 519-528 (1994)).

### Sialyltransferases

Sialyltransferases are another type of glycosyltransferase that is useful in the recombinant cells and reaction mixtures of the invention. Cells that produce recombinant sialyltransferases will also produce CMP-sialic acid, which is a sialic acid donor for sialyltransferases. Examples of sialyltransferases that are suitable for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalNAc III (the sialyltransferase nomenclature used herein is as described in Tsuji et al., Glycobiology 6: v-xiv (1996)). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem. 256: 3159 (1981), Weinstein et al., J. Biol. Chem. 257: 13845 (1982) and Wen et al., J. Biol. Chem. 267: 21011 (1992). Another exemplary α2,3-sialyltransferase (EC 2.4.99.4) transfers sialic acid to the non-reducing terminal Gal of the disaccharide or glycoside. see, Rearick et al., J. Biol. Chem. 254: 4444 (1979) and Gillespie et al., J. Biol. Chem. 267: 21004 (1992). Further exemplary enzymes include Gal-β-1,4-GlcNAc α-2,6 sialyltransferase (See, Kurosawa et al. Eur. J. Biochem. 219: 375-381 (1994)).

Preferably, for glycosylation of carbohydrates of glycopeptides the sialyltransferase will be able to transfer sialic acid to the sequence Galβ1,4GlcNAc-, the most common penultimate sequence underlying the terminal sialic acid on fully sialylated carbohydrate structures (see, Table 1).

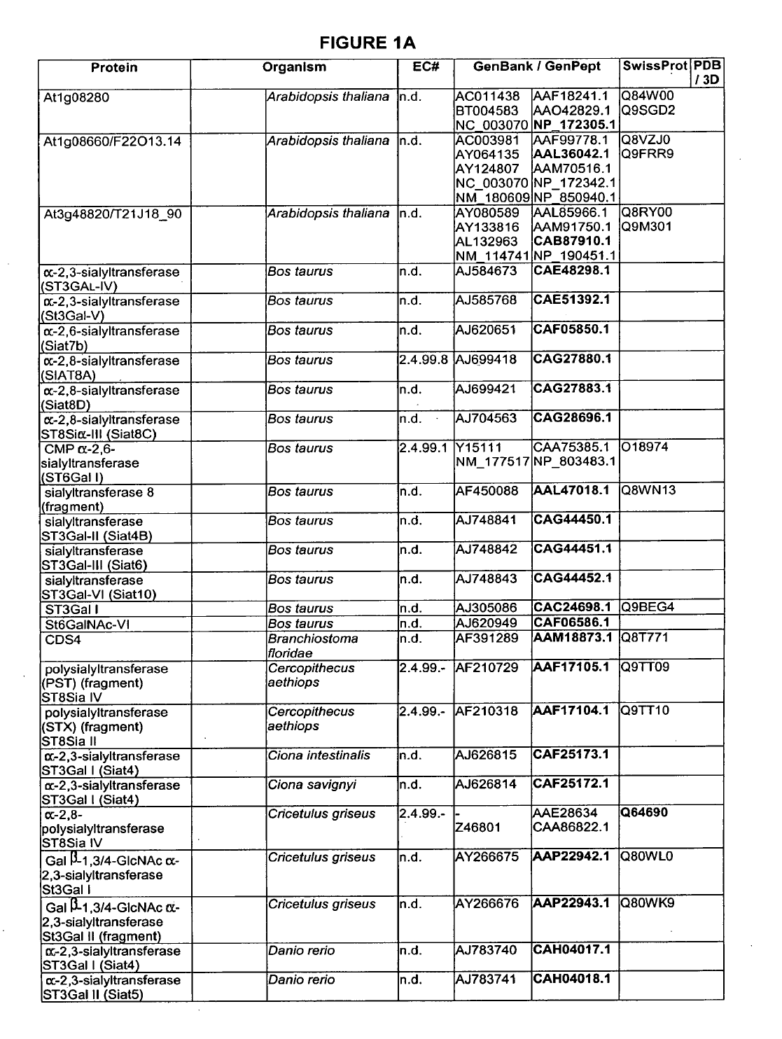

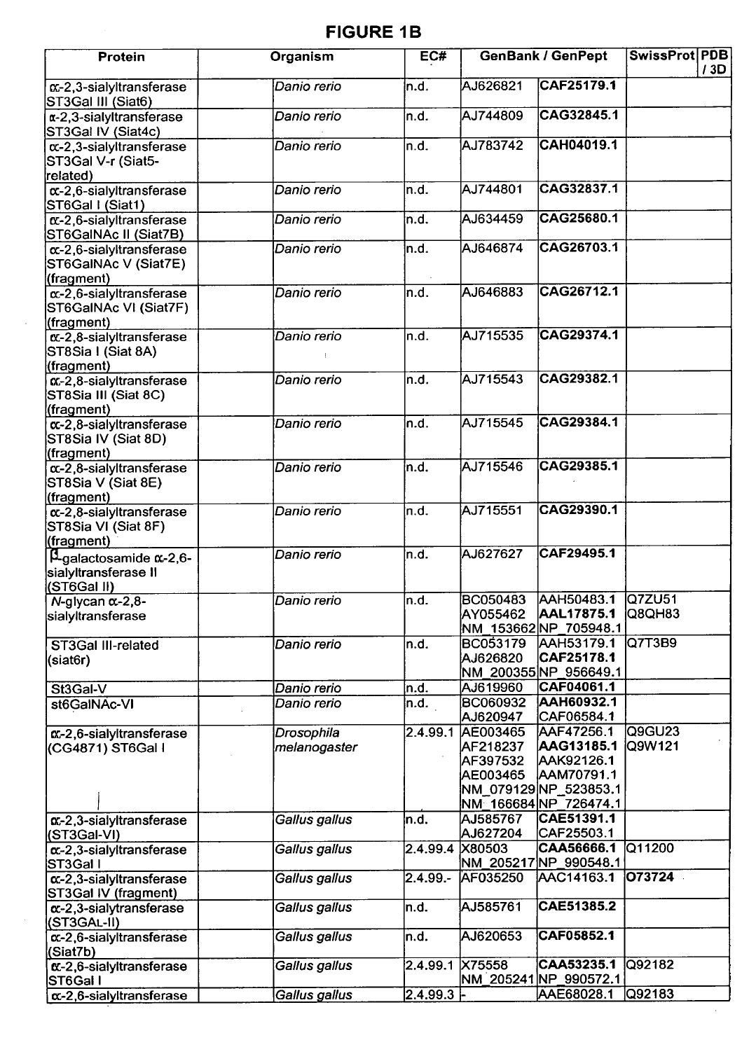

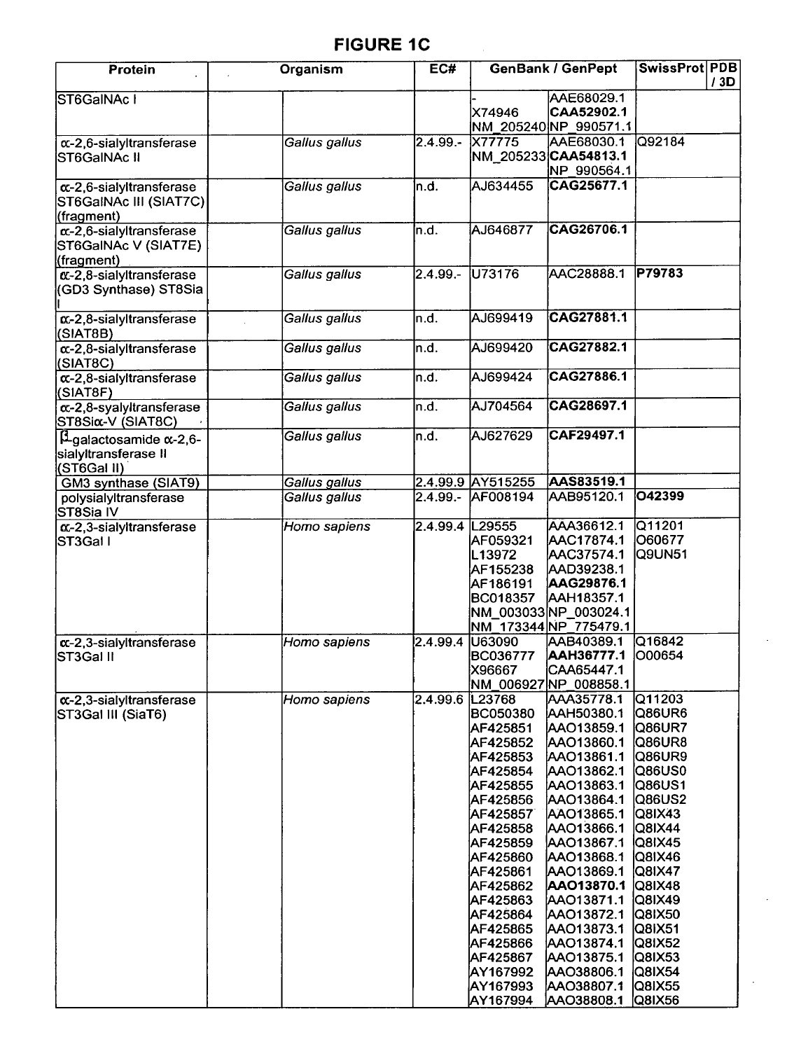

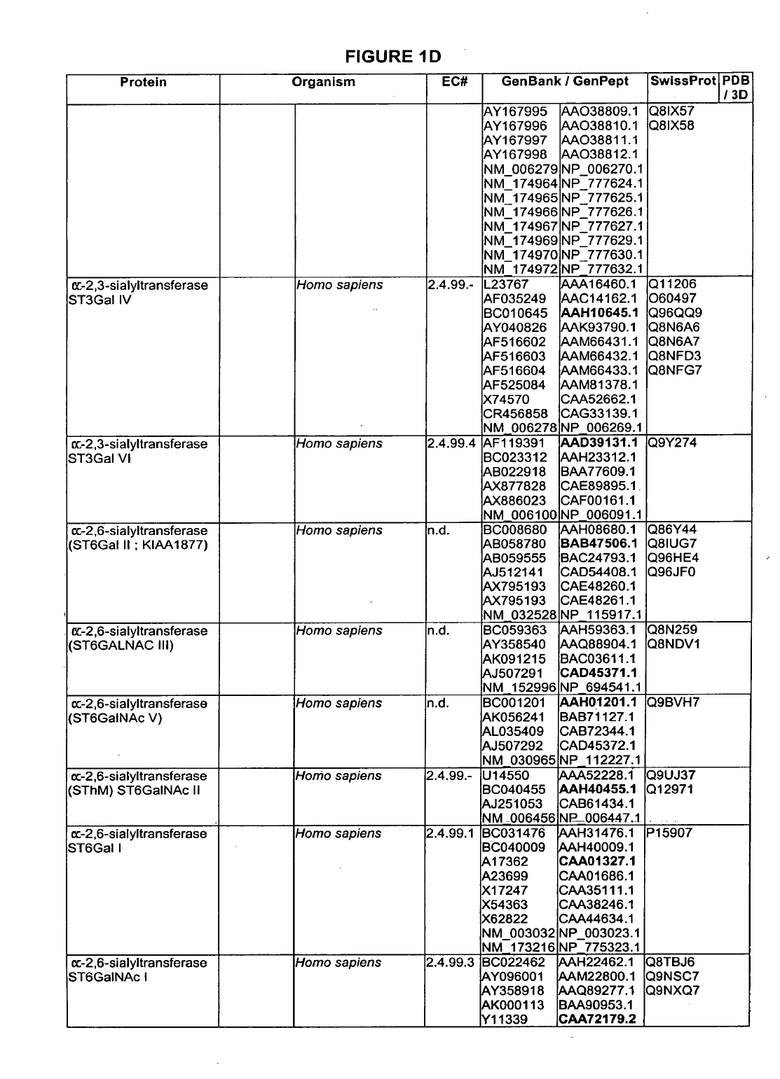

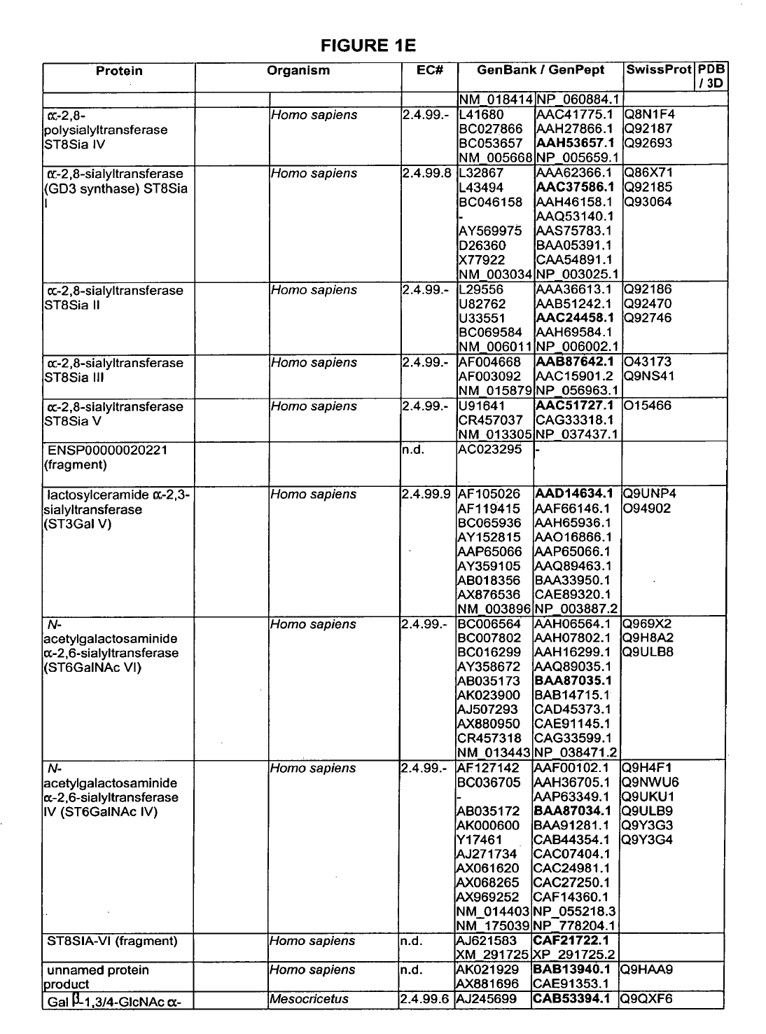

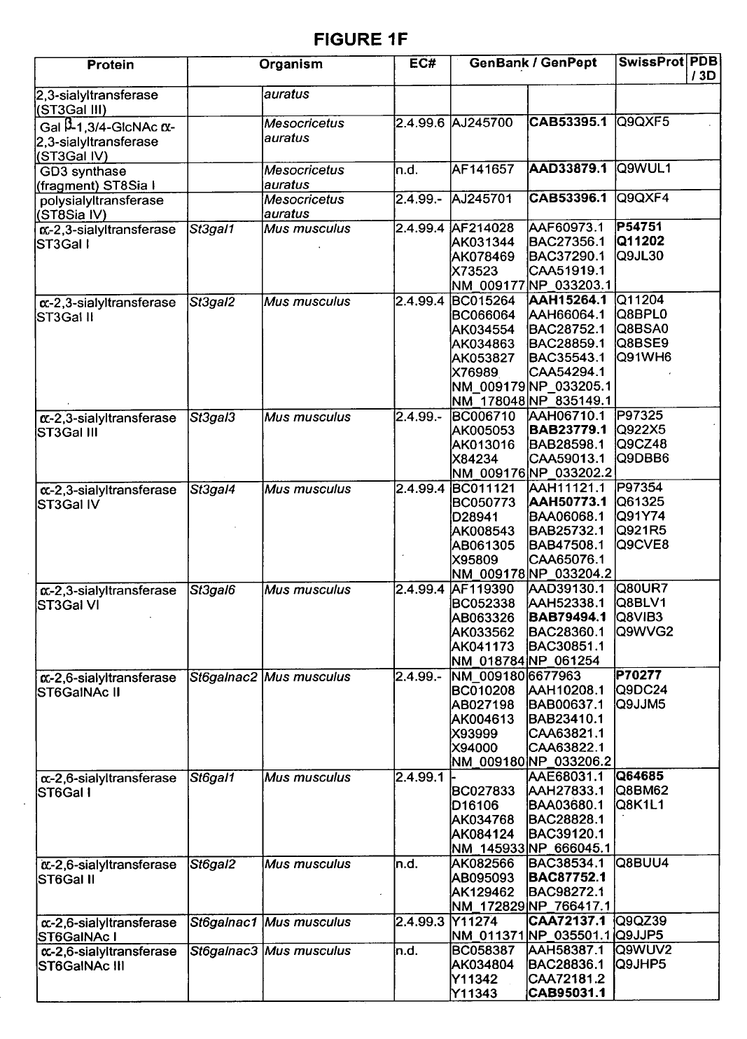

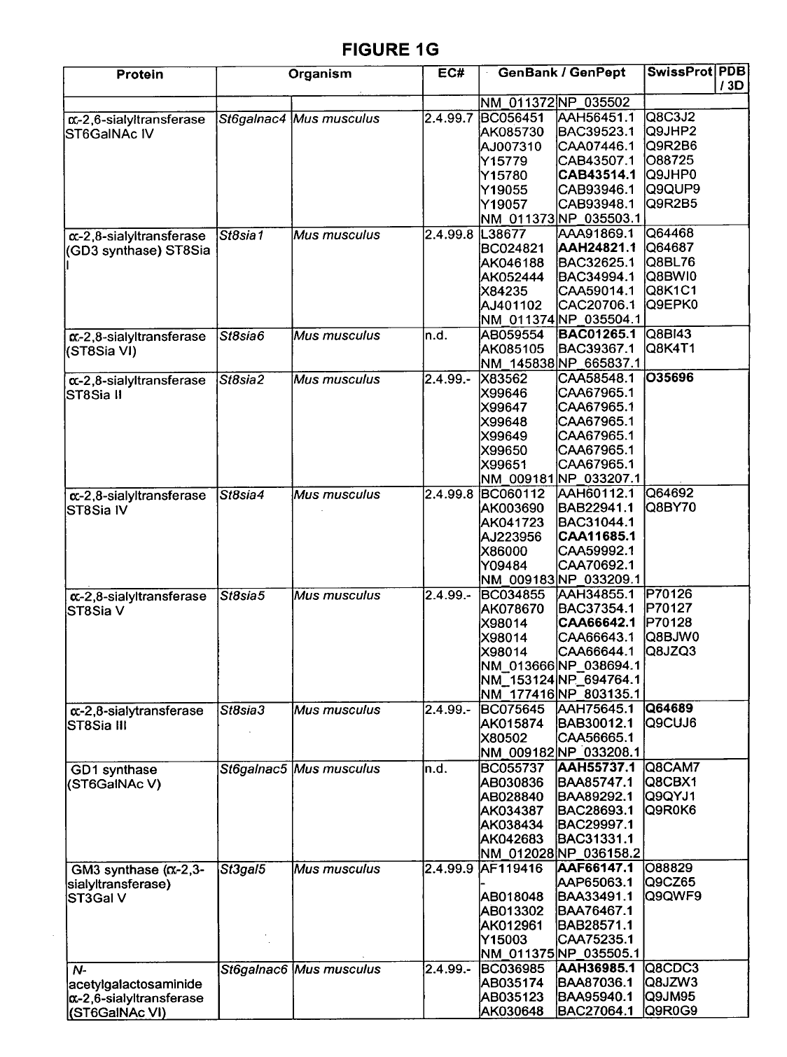

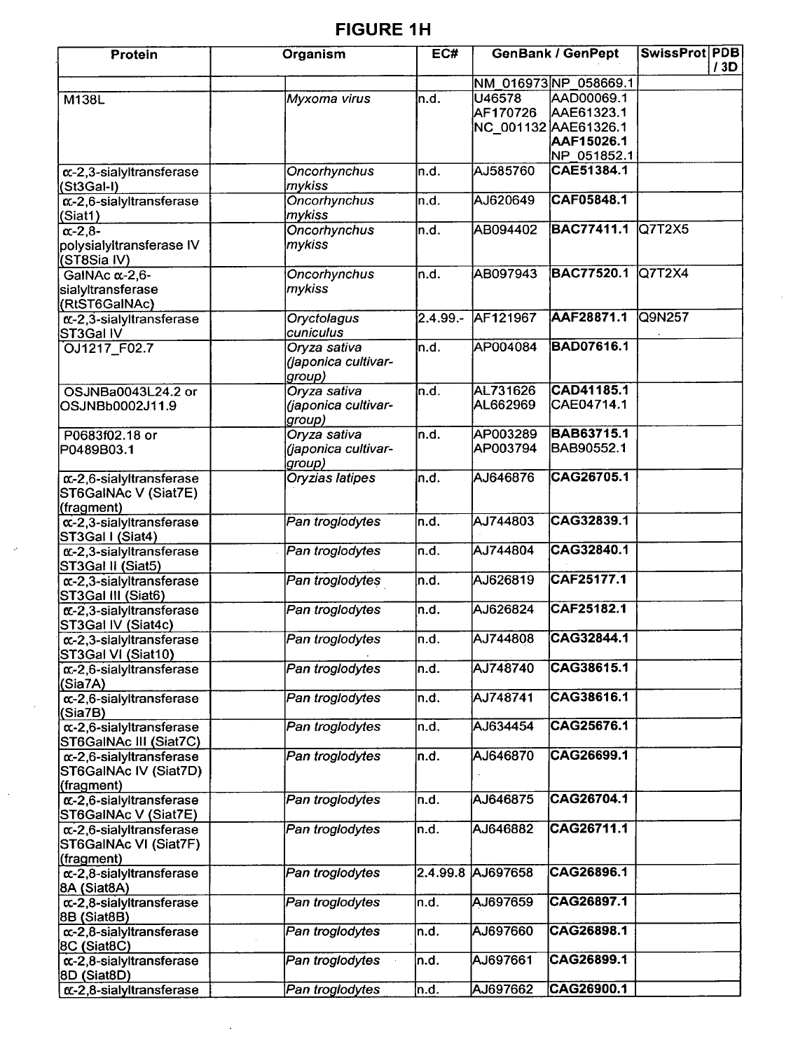

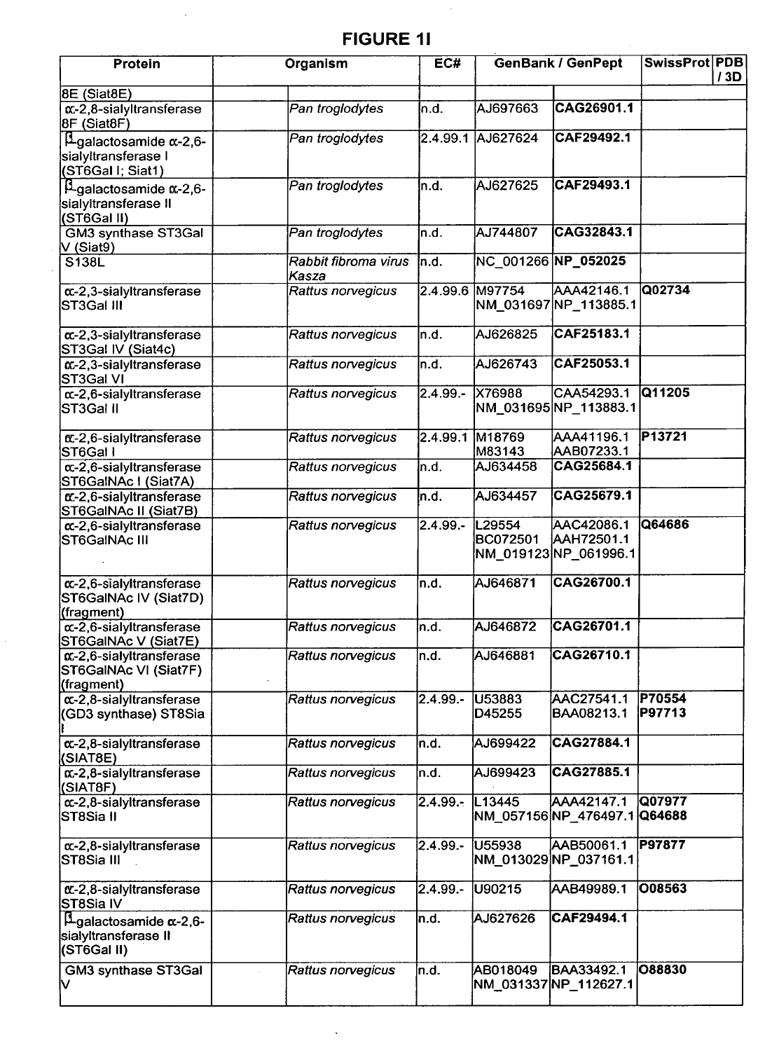

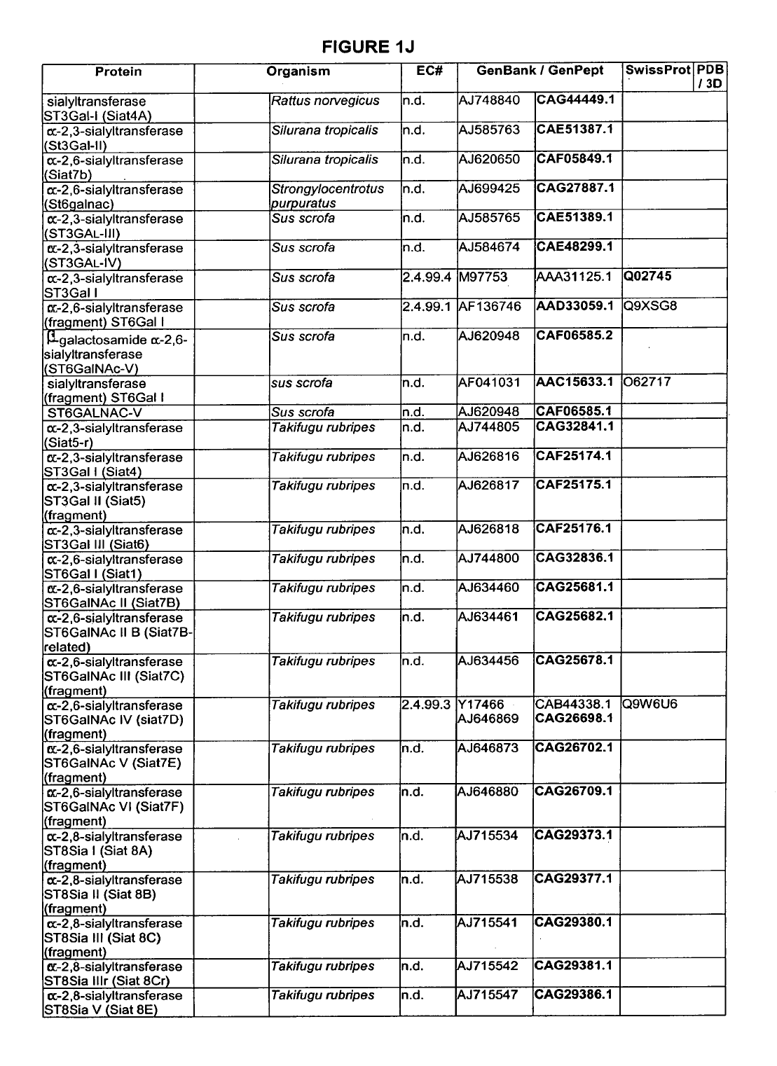

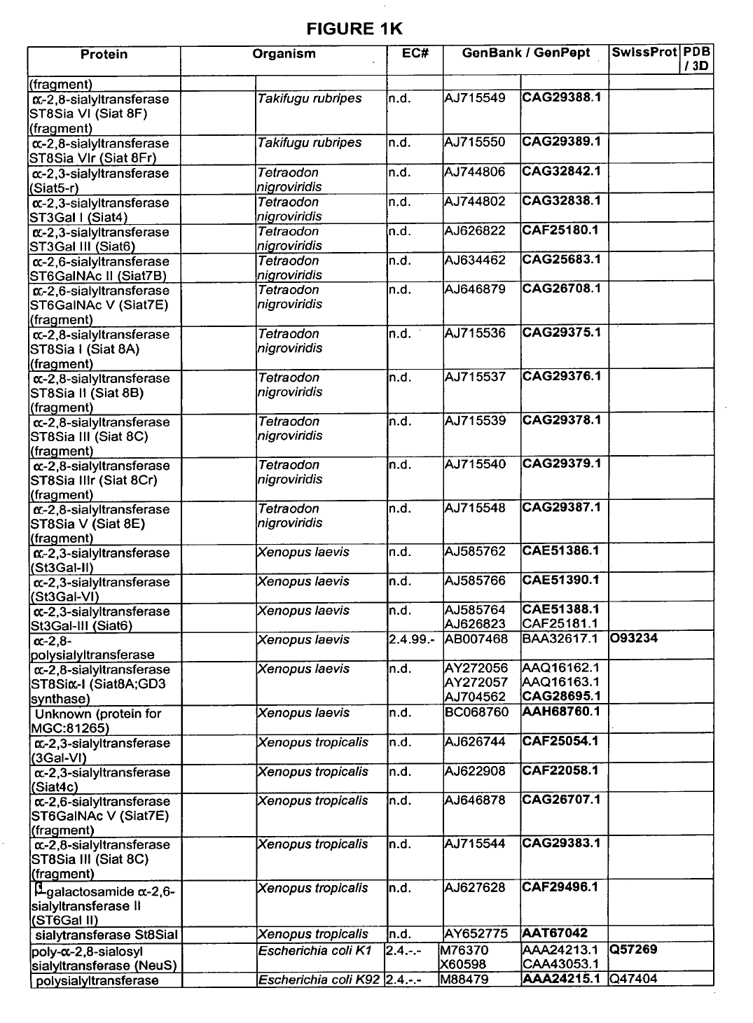

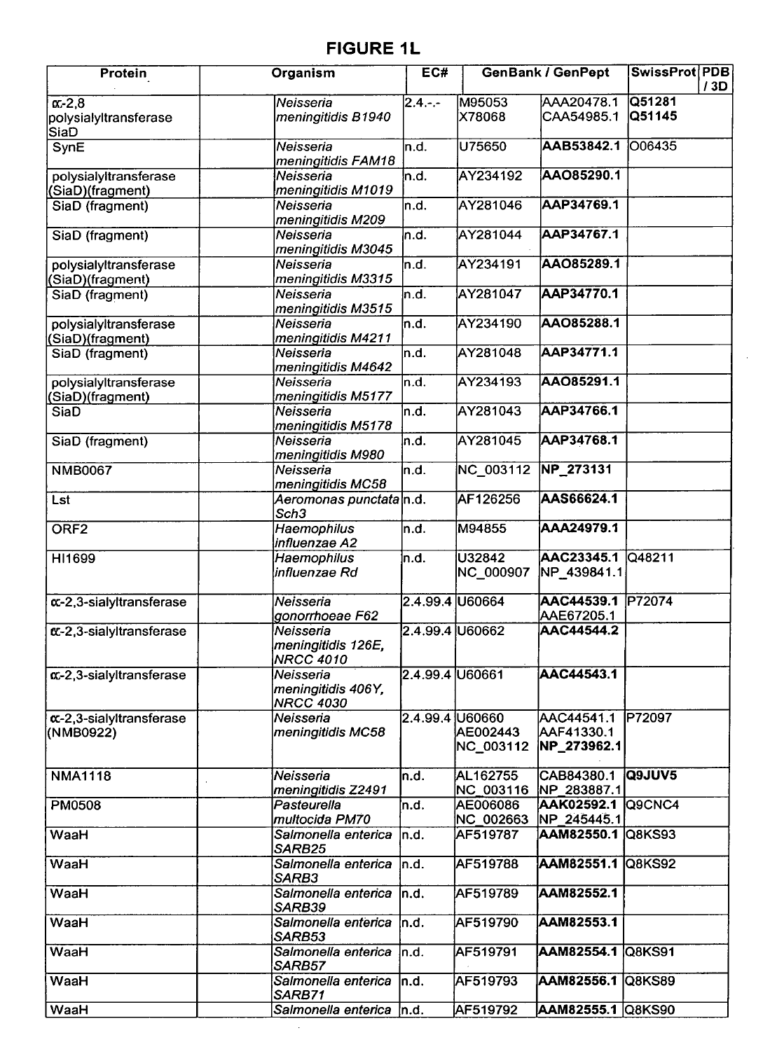

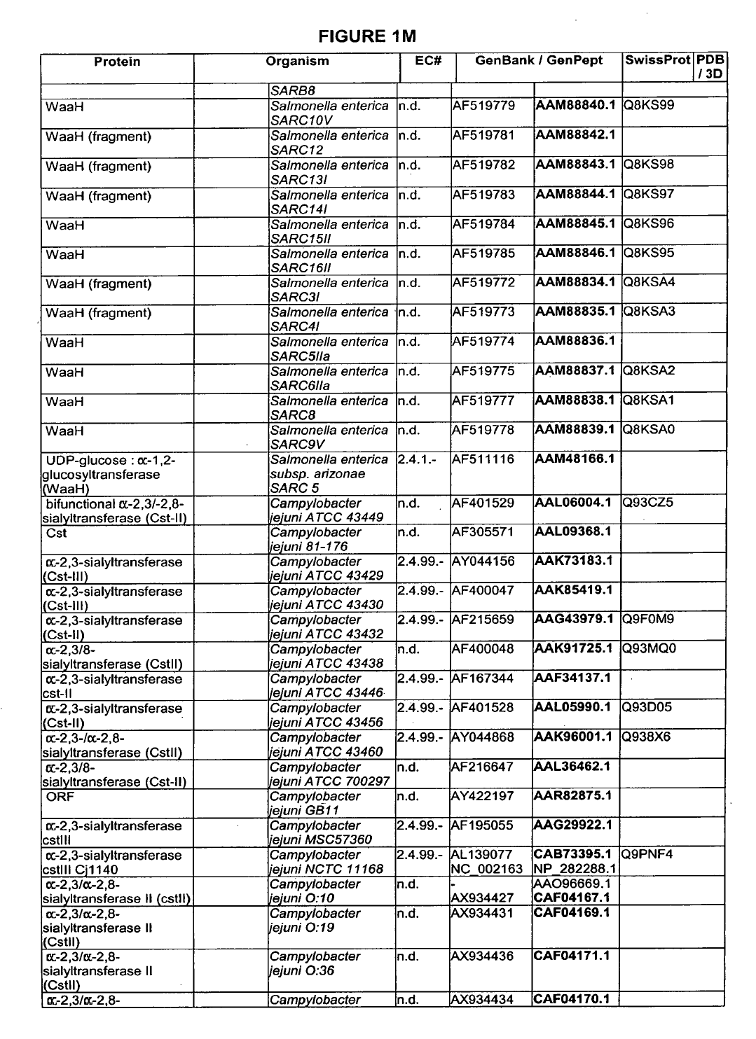

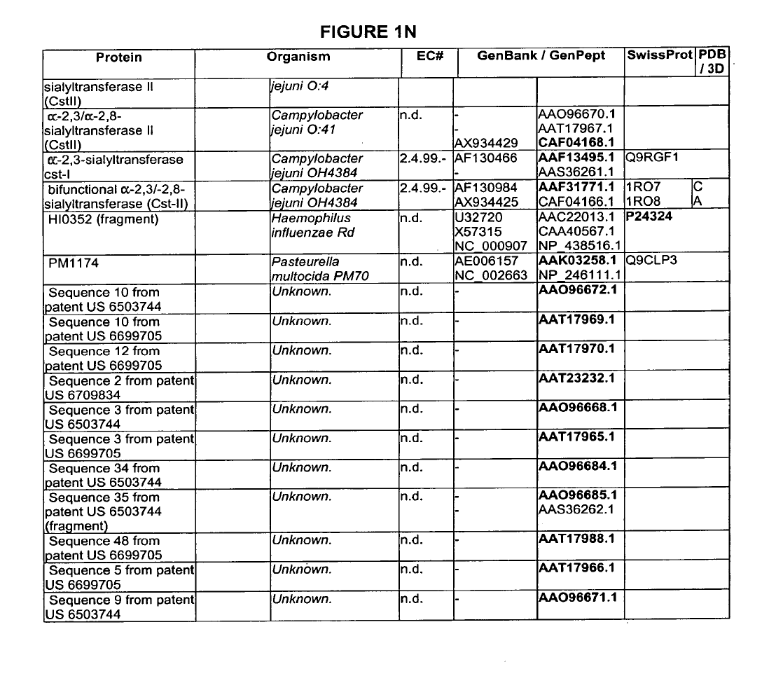

Eukaryotic sialyltransferases can also be used in the invention. Examples of suitable eukaryotic sialyltransferases for use in the present invention include ST3Gal III (e.g., a rat or human ST3Gal III), ST3Gal IV, ST3Gal I, ST6Gal I, ST3Gal V, ST6Gal II, ST6GalNAc I, ST6GalNAc II, and ST6GalN Ac III (the sialyltransferase nomenclature used herein is as described in Tsuji et al. (1996) Glycobiology 6: v-xiv). An exemplary α(2,3)sialyltransferase referred to as α(2,3)sialyltransferase (EC 2.4.99.6) transfers sialic acid to the non-reducing terminal Gal of a Galβ1→3Glc disaccharide or glycoside. See, Van den Eijnden et al., J. Biol. Chem., 256:3159 (1981), Weinstein et al., J. Biol. Chem., 257:13845 (1982) and Wen et al., J. Biol. Chem., 267:21011 (1992). Another exemplary α-2,3-sialyltransferase (EC 2.4.99.4) transfers sialic acid to the non-reducing terminal Gal of the disaccharide or glycoside. See, Rearick et al., J. Biol. Chem., 254:4444 (1979) and Gillespie et al, J. Biol. Chem., 267:21004 (1992). Further exemplary enzymes include Gal-β-1,4-GlcNAc α-2,6 sialyltransferase (See, Kurosawa et al. Eur. J. Biochem. 219: 375-381 (1994)). Eukaryotic sialyltransferases generally comprise different functional domains, e.g., a cytoplasmic domain, a signal-anchor domain, a stem region and a catalytic domain. In preferred embodiments, the catalytic domain of a eukaryotic sialyltransferase is expressed in a host cell. Other sialyltransferases that can be used in the invention are found in Table 1 and FIG. 1, below.

TABLE 1

| Sialyltransferase | Accession number |
|---|---|
| ST3Gal I | X73523 |
| ST3Gal II | BC015264 |
| ST3Gal II | X76989 |
| ST3Gal III | BC006710 |
| ST3Gal IV | BC011121 |
| ST3Gal V | AF119416 |
| ST3Gal VI | NM_018784 |
| ST6Gal I | BB768706 |
| ST6Gal I | BB768706 |
| ST6Gal I | D16106 |
| ST6GalNAc I | NM_011371 |
| ST6GalNAc I | NM_011371 |
| ST6GalNAc II | X93999 |
| ST6GalNAc III | Y11342 |
| ST6GalNAc IV | NM_011373 |
| ST6GalNAc IV | Y15779 |
| ST6GalNAc IV | Y15779 |
| ST6GalNAc V | AB028840 |
| ST6GalNAc VI | AB035123 |
| ST6GalNAc VI | AV101836 |
| ST6GalNAc VI | BB772604 |
| ST8Sia I | AW490593 |
| ST8Sia I | NM_011374 |
| ST8Sia II | X83562 |
| ST8Sia II | X83562 |
| ST8Sia III | X80502 |
| ST8Sia IV | X86000 |
| ST8Sia V | X98014 |
| ST8Sia VI | AB059554 |

In addition to the sialyltransferases listed in Tables 3 and 4, the invention also includes use of the following sialyltransferases: protein encoded by the siaA protein of Haemophilus influenzae, accession number AAL38659; an α-2,6-sialyltransferase gene from Photobacterium damsela, accession number BAA25316; protein from Pasteurella multocida, accession number NP_{—}245125; and protein from Haemophilus ducreyi, accession number NP_{—}872679.

An example of a sialyltransferase that is useful in the claimed methods is ST3Gal III, which is also referred to as α(2,3)sialyltransferase (EC 2.4.99.6). This enzyme catalyzes the transfer of sialic acid to the Gal of a Galβ1,3GlcNAc or Galβ1,4GlcNAc glycoside (see, e.g., Wen et al., J. Biol. Chem. 267: 21011 (1992); Van den Eijnden et al., J. Biol. Chem. 256: 3159 (1991)) and is responsible for sialylation of asparagine-linked oligosaccharides in glycopeptides. The sialic acid is linked to a Gal with the formation of an α-linkage between the two saccharides. Bonding (linkage) between the saccharides is between the 2-position of NeuAc and the 3-position of Gal. This particular enzyme can be isolated from rat liver (Weinstein et al., J. Biol. Chem. 257: 13845 (1982)); the human cDNA (Sasaki et al. (1993) J. Biol. Chem. 268: 22782-22787; Kitagawa & Paulson (1994) J. Biol. Chem. 269: 1394-1401) and genomic (Kitagawa et al. (1996) J. Biol. Chem. 271: 931-938) DNA sequences are known, facilitating production of this enzyme by recombinant expression. In a preferred embodiment, the claimed sialylation methods use a rat ST3Gal III.

Other exemplary sialyltransferases of use in the present invention include those isolated from Campylobacter jejuni, including the α(2,3). See, e.g., WO99/49051.

Sialyltransferases other those listed in Table 1, are also useful in an economic and efficient large-scale process for sialylation of commercially important glycopeptides. See, for example, the work of W. Wakarchuk generally and, specifically, U.S. Pat. Nos. 6,709,834; 6,699,705; 6,689,604; 6,210,933; and 6,096,529; and published U.S. Patent Application No.s 2004/0152165; 2003/0148459; 2002/0042369.

As a simple test to find out the utility of these other enzymes, various amounts of each enzyme (1-100 mU/mg protein) are reacted with asialo-α₁ AGP (at 1-10 mg/ml) to compare the ability of the sialyltransferase of interest to sialylate glycopeptides relative to either bovine ST6Gal I, ST3Gal III or both sialyltransferases. Alternatively, other glycopeptides or glycopeptides, or N-linked oligosaccharides enzymatically released from the peptide backbone can be used in place of asialo-α₁ AGP for this evaluation. Sialyltransferases with the ability to sialylate N-linked oligosaccharides of glycopeptides more efficiently than ST6Gal I are useful in a practical large-scale process for peptide sialylation.

### GalNAc Transferases

N-acetylgalactosaminyltransferases are of use in practicing the present invention, particularly for binding a GalNAc moiety to an amino acid of the O-linked glycosylation site of the peptide. Suitable N-acetylgalactosaminyltransferases include, but are not limited to, α(1,3) N-acetylgalactosaminyltransferase, β(1,4) N-acetylgalactosaminyltransferases (Nagata et al., J. Biol. Chem. 267: 12082-12089 (1992) and Smith et al., J. Biol. Chem. 269: 15162 (1994)) and polypeptide N-acetylgalactosaminyltransferase (Roma et al., J. Biol. Chem. 268: 12609 (1993)). See also the work of W. Wakarchuk generally and U.S. Pat. No. 6,723,545; and published U.S. Patent Application No. 2003/0180928; 2003/0157658; 2003/0157657; and 2003/0157656.

Production of proteins such as the enzyme GalNAc T_{I-XX} from cloned genes by genetic engineering is well known. See, e.g., U.S. Pat. No. 4,761,371. One method involves collection of sufficient samples, then the amino acid sequence of the enzyme is determined by N-terminal sequencing. This information is then used to isolate a cDNA clone encoding a full-length (membrane bound) transferase which upon expression in the insect cell line Sf9 resulted in the synthesis of a fully active enzyme. The acceptor specificity of the enzyme is then determined using a semiquantitative analysis of the amino acids surrounding known glycosylation sites in 16 different proteins followed by in vitro glycosylation studies of synthetic peptides. This work has demonstrated that certain amino acid residues are overrepresented in glycosylated peptide segments and that residues in specific positions surrounding glycosylated serine and threonine residues may have a more marked influence on acceptor efficiency than other amino acid moieties.

### Cell-Bound Glycosyltransferases

In another embodiment, the enzymes utilized in the method of the invention are cell-bound glycosyltransferases. Although many soluble glycosyltransferases are known (see, for example, U.S. Pat. No. 5,032,519), glycosyltransferases are generally in membrane-bound form when associated with cells. Many of the membrane-bound enzymes studied thus far are considered to be intrinsic proteins; that is, they are not released from the membranes by sonication and require detergents for solubilization. Surface glycosyltransferases have been identified on the surfaces of vertebrate and invertebrate cells, and it has also been recognized that these surface transferases maintain catalytic activity under physiological conditions. However, the more recognized function of cell surface glycosyltransferases is for intercellular recognition (Roth, MOLECULAR APPROACHES to SUPRACELLULAR PHENOMENA, 1990).

Methods have been developed to alter the glycosyltransferases expressed by cells. For example, Larsen et al., Proc. Natl. Acad. Sci. USA 86: 8227-8231 (1989), report a genetic approach to isolate cloned cDNA sequences that determine expression of cell surface oligosaccharide structures and their cognate glycosyltransferases. A cDNA library generated from mRNA isolated from a murine cell line known to express UDP-galactose:.β.-D-galactosyl-1,4-N-acetyl-D-glucosaminide α-1,3-galactosyltransferase was transfected into COS-1 cells. The transfected cells were then cultured and assayed for a 1-3 galactosyltransferase activity.

Francisco et al., Proc. Natl. Acad. Sci. USA 89: 2713-2717 (1992), disclose a method of anchoring (3-lactamase to the external surface of Escherichia coli. A tripartite fusion consisting of (i) a signal sequence of an outer membrane protein, (ii) a membrane-spanning section of an outer membrane protein, and (iii) a complete mature β-lactamase sequence is produced resulting in an active surface bound β-lactamase molecule. However, the Francisco method is limited only to procaryotic cell systems and as recognized by the authors, requires the complete tripartite fusion for proper functioning.

### Sulfotransferases

The invention also provides methods for producing peptides that include sulfated molecules, including, for example sulfated polysaccharides such as heparin, heparan sulfate, carragenen, and related compounds. Suitable sulfotransferases include, for example, chondroitin-6-sulphotransferase (chicken cDNA described by Fukuta et al., J. Biol. Chem. 270: 18575-18580 (1995); GenBank Accession No. D49915), glycosaminoglycan N-acetylglucosamine N-deacetylase/N-sulphotransferase 1 (Dixon et al., Genomics 26: 239-241 (1995); UL18918), and glycosaminoglycan N-acetylglucosamine N-deacetylase/N-sulphotransferase 2 (murine cDNA described in Orellana et al., J. Biol. Chem. 269: 2270-2276 (1994) and Eriksson et al., J. Biol. Chem. 269: 10438-10443 (1994); human cDNA described in GenBank Accession No. U2304).

### Glycosidases

This invention also encompasses the use of wild-type and mutant glycosidases. Mutant β-galactosidase enzymes have been demonstrated to catalyze the formation of disaccharides through the coupling of an α-glycosyl fluoride to a galactosyl acceptor molecule. (Withers, U.S. Pat. No. 6,284,494; issued Sep. 4, 2001). Other glycosidases of use in this invention include, for example, β-glucosidases, β-galactosidases, β-mannosidases, β-acetyl glucosaminidases, β-N-acetyl galactosaminidases, β-xylosidases, β-fucosidases, cellulases, xylanases, galactanases, mannanases, hemicellulases, amylases, glucoamylases, α-glucosidases, α-galactosidases, α-mannosidases, α-N-acetyl glucosaminidases, α-N-acetyl galactose-aminidases, α-xylosidases, α-fucosidases, and neuraminidases/sialidases.

### Immobilized Enzymes

The present invention also provides for the use of enzymes that are immobilized on a solid and/or soluble support. In an exemplary embodiment, there is provided a glycosyltransferase that is conjugated to a PEG via an intact glycosyl linker according to the methods of the invention. The PEG-linker-enzyme conjugate is optionally attached to solid support. The use of solid supported enzymes in the methods of the invention simplifies the work up of the reaction mixture and purification of the reaction product, and also enables the facile recovery of the enzyme. The glycosyltransferase conjugate is utilized in the methods of the invention. Other combinations of enzymes and supports will be apparent to those of skill in the art.

### Enzyme Production

### Acquisition of Enzyme Coding Sequences

### General Recombinant Technology

This invention relies on routine techniques in the field of recombinant genetics. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of the cloned wild-type enzyme genes, synthetic oligonucleotides, and polynucleotides encoding endoglycoceramide synthases can be verified after cloning using, e.g., the chain termination method for sequencing double-stranded templates of Wallace et al., Gene 16: 21-26 (1981).

### Cloning and Subcloning of a Wild-Type Enzyme Coding Sequence

A number of polynucleotide sequences encoding wild-type enzymes, e.g., GenBank Accession No. U39554, have been determined and can be synthesized or obtained from a commercial supplier, such as Blue Heron Biotechnology (Bothell, Wash.).

The rapid progress in the studies of human genome has made possible a cloning approach where a human DNA sequence database can be searched for any gene segment that has a certain percentage of sequence homology to a known nucleotide sequence, such as one encoding a previously identified enzyme. Any DNA sequence so identified can be subsequently obtained by chemical synthesis and/or a polymerase chain reaction (PCR) technique such as overlap extension method. For a short sequence, completely de novo synthesis may be sufficient; whereas further isolation of full length coding sequence from a human cDNA or genomic library using a synthetic probe may be necessary to obtain a larger gene.

Alternatively, a nucleic acid sequence encoding an enzyme can be isolated from a cDNA or genomic DNA library using standard cloning techniques such as polymerase chain reaction (PCR), where homology-based primers can often be derived from a known nucleic acid sequence encoding an enzyme. Most commonly used techniques for this purpose are described in standard texts, e.g., Sambrook and Russell, supra.

cDNA libraries suitable for obtaining a coding sequence for a wild-type enzyme may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate the full length polynucleotide sequence encoding the wild-type enzyme from the cDNA library. A general description of appropriate procedures can be found in Sambrook and Russell, supra.

A similar procedure can be followed to obtain a full length sequence encoding a wild-type enzyme from a genomic library. Genomic libraries are commercially available or can be constructed according to various art-recognized methods. In general, to construct a genomic library, the DNA is first extracted from an organism where an enzyme is likely found. The DNA is then either mechanically sheared or enzymatically digested to yield fragments of about 12-20 kb in length. The fragments are subsequently separated by gradient centrifugation from polynucleotide fragments of undesired sizes and are inserted in bacteriophage λ vectors. These vectors and phages are packaged in vitro. Recombinant phages are analyzed by plaque hybridization as described in Benton and Davis, Science, 196: 180-182 (1977). Colony hybridization is carried out as described by Grunstein et al., Proc. Natl. Acad. Sci. USA, 72: 3961-3965 (1975).

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, the full length nucleic acid encoding a wild-type enzyme is obtained.

Upon acquiring a nucleic acid sequence encoding a wild-type enzyme, the coding sequence can be subcloned into a vector, for instance, an expression vector, so that a recombinant enzyme can be produced from the resulting construct. Further modifications to the wild-type enzyme coding sequence, e.g., nucleotide substitutions, may be subsequently made to alter the characteristics of the enzyme.

Introducing Mutations into the Enzyme Coding Sequence

Modifications altering the enzymatic activity of an enzyme may be made in various locations within the polynucleotide coding sequence. The preferred locations for such modifications are, however, within the active site of the enzyme. A conserved sequence encoding a three-amino acid segment Asn-Glu-Pro was previously identified within the active site of enzymes, and the Glu residue within the segment appears to be connected to the activity of the enzyme (Sakaguchi et al., Biochem. Biophys. Res. Commun., 1999, 260: 89-93)

From an encoding nucleic acid sequence, the amino acid sequence of a wild-type enzyme, e.g., SEQ ID NO:1 or 2, can be deduced and the presence of an active site can be confirmed. Preferably, mutations are introduced into the active site. For instance, the Glu residue at position 233 of SEQ ID NO:1 or position 224 of SEQ ID NO:2, both located in the middle of a three-amino acid segment Asn-Glu-Pro, can be targeted for mutation, such as deletion or substitution by another amino acid residue. In addition, other Glu residues, e.g., the Glu located at position 351 of SEQ ID NO:1 or position 343 of SEQ ID NO:2, are also targets for introducing mutations to alter the enzymatic activity of an enzyme. An artisan can accomplish the goal of mutating a target Glu residue by employing any one of the well known mutagenesis methods, which are discussed in detail below. Exemplary modifications are introduced to replace the Glu residue with another amino acid residue as depicted in SEQ ID NOs:3-7.

A variety of diversity-generating protocols are established and described in the art. See, e.g., Zhang et al., Proc. Natl. Acad. Sci. USA, 94: 4504-4509 (1997); and Stemmer, Nature, 370: 389-391 (1994). The procedures can be used separately or in combination to produce variants of a set of nucleic acids, and hence variants of encoded polypeptides. Kits for mutagenesis, library construction, and other diversity-generating methods are commercially available.

Mutational methods of generating diversity include, for example, site-directed mutagenesis (Botstein and Shortie, Science, 229: 1193-1201 (1985)), mutagenesis using uracil-containing templates (Kunkel, Proc. Natl. Acad. Sci. USA, 82: 488-492 (1985)), oligonucleotide-directed mutagenesis (Zoller and Smith, Nucl. Acids Res., 10: 6487-6500 (1982)), phosphorothioate-modified DNA mutagenesis (Taylor et al., Nucl. Acids Res., 13: 8749-8764 and 8765-8787 (1985)), and mutagenesis using gapped duplex DNA (Kramer et al., Nucl. Acids Res., 12: 9441-9456 (1984)).

Other possible methods for generating mutations include point mismatch repair (Kramer et al., Cell, 38: 879-887 (1984)), mutagenesis using repair-deficient host strains (Carter et al., Nucl. Acids Res., 13: 4431-4443 (1985)), deletion mutagenesis (Eghtedarzadeh and Henikoff, Nucl. Acids Res., 14: 5115 (1986)), restriction-selection and restriction-purification (Wells et al., Phil. Trans. R. Soc. Lond. A, 317: 415-423 (1986)), mutagenesis by total gene synthesis (Nambiar et al., Science, 223: 1299-1301 (1984)), double-strand break repair (Mandecki, Proc. Natl. Acad. Sci. USA, 83: 7177-7181 (1986)), mutagenesis by polynucleotide chain termination methods (U.S. Pat. No. 5,965,408), and error-prone PCR (Leung et al., Biotechniques, 1: 11-15 (1989)).

At the completion of modification, the mutant enzyme coding sequences can then be subcloned into an appropriate vector for recombinant production in the same manner as the wild-type genes.

### Modification of Nucleic Acids for Preferred Codon Usage in a Host Organism

The polynucleotide sequence encoding an enzyme (either wild-type or mutant) can be altered to coincide with the preferred codon usage of a particular host. For example, the preferred codon usage of one strain of bacteria can be used to derive a polynucleotide that encodes a mutant enzyme of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell. U.S. Pat. No. 5,824,864, for example, provides the frequency of codon usage by highly expressed genes exhibited by dicotyledonous plants and monocotyledonous plants.

The sequences of the cloned enzyme genes, synthetic polynucleotides, and modified enzyme genes can be verified using, e.g., the chain termination method for sequencing double-stranded templates as described in Wallace et al., Gene 16:21-26 (1981).

### Expression and Purification of the Enzymes

Following sequence verification, the wild-type or mutant enzyme of the present invention can be produced using routine techniques in the field of recombinant genetics, relying on the polynucleotide sequences encoding the polypeptide disclosed herein.

### Expression Systems

To obtain high level expression of a nucleic acid encoding a wild-type or a mutant enzyme of the present invention, one typically subclones a polynucleotide encoding the enzyme into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the wild-type or mutant enzyme are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the enzyme in host cells. A typical expression cassette thus contains a promoter operably linked to the nucleic acid sequence encoding the wild-type or mutant enzyme and signals required for efficient polyadenylation of the transcript, ribosome binding sites, and translation termination. The nucleic acid sequence encoding the enzyme is typically linked to a cleavable signal peptide sequence to promote secretion of the enzyme by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMT010/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the mutant enzyme under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

As discussed above, a person skilled in the art will recognize that various conservative substitutions can be made to any wild-type or mutant enzyme or its coding sequence while still retaining the synthetic activity of the enzyme. Moreover, modifications of a polynucleotide coding sequence may also be made to accommodate preferred codon usage in a particular expression host without altering the resulting amino acid sequence.

### Transfection Methods

Standard transfection methods are used to produce bacterial, mammalian, yeast or insect cell lines that express large quantities of the wild-type or mutant enzyme, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the wild-type or mutant enzyme.

### Detection of the Expression of Recombinant Enzymes

After the expression vector is introduced into appropriate host cells, the transfected cells are cultured under conditions favoring expression of the wild-type or mutant enzyme. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding an enzyme in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a wild-type or mutant enzyme of the present invention, such as a polypeptide having the amino acid sequence of SEQ ID NO:3, 4, or 5, (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the recombinant polypeptide or an antigenic portion thereof. The methods of raising polyclonal and monoclonal antibodies are well established and their descriptions can be found in the literature, see, e.g., Harlow and Lane, supra; Kohler and Milstein, Eur. J. Immunol., 6: 511-519 (1976). More detailed descriptions of preparing antibody against the mutant enzyme of the present invention and conducting immunological assays detecting the mutant enzyme are provided in a later section.

In addition, functional assays may also be performed for the detection of a recombinant enzyme in transfected cells. Assays for detecting hydrolytic or synthetic activity of the recombinant enzyme are generally described in a later section.

### Purification of Recombinant Enzymes

Once the expression of a recombinant enzyme in transfected host cells is confirmed, the host cells are then cultured in an appropriate scale for the purpose of purifying the recombinant enzyme.

Purification of Recombinant Polypeptides from Bacteria

When the enzymes of the present invention are produced recombinantly by transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify recombinant polypeptides, e.g., a mutant enzyme, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### Standard Protein Separation Techniques for Purification

When a recombinant polypeptide, e.g., the mutant enzyme of the present invention, is expressed in host cells in a soluble form, its purification can follow the standard protein purification procedure described below.

### Solubility Fractionation

Often as an initial step, and if the protein mixture is complex, an initial salt fractionation can separate many of the unwanted host cell proteins (or proteins derived from the cell culture media) from the recombinant protein of interest, e.g., a mutant enzyme of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a mutant enzyme. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### Column Chromatography

The proteins of interest (such as the mutant enzyme of the present invention) can also be separated from other proteins on the basis of their size, net surface charge, hydrophobicity, or affinity for ligands. In addition, antibodies raised against enzyme can be conjugated to column matrices and the enzyme immunopurified. All of these methods are well known in the art.

It will be apparent to one of skill that chromatographic techniques can be performed at any scale and using equipment from many different manufacturers (e.g., Pharmacia Biotech).

### Enzyme Assays

### Production of Antibodies Against Enzymes and Immunoassays for Detection of Enzyme Expression

To confirm the production of a recombinant enzyme, immunological assays may be useful to detect in a sample the expression of the enzyme. Immunological assays are also useful for quantifying the expression level of the recombinant enzyme.

### Production of Antibodies Against Enzyme

Methods for producing polyclonal and monoclonal antibodies that react specifically with an immunogen of interest are known to those of skill in the art (see, e.g., Coligan, Current Protocols in Immunology Wiley/Greene, N.Y., 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256: 495-497, 1975). Such techniques include antibody preparation by selection of antibodies from libraries of recombinant antibodies in phage or similar vectors (see, Huse et al., Science 246: 1275-1281, 1989; and Ward et al., Nature 341: 544-546, 1989).

In order to produce antisera containing antibodies with desired specificity, the polypeptide of interest (e.g., a mutant enzyme of the present invention) or an antigenic fragment thereof can be used to immunize suitable animals, e.g., mice, rabbits, or primates. A standard adjuvant, such as Freund's adjuvant, can be used in accordance with a standard immunization protocol. Alternatively, a synthetic antigenic peptide derived from that particular polypeptide can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. Further fractionation of the antisera to enrich antibodies specifically reactive to the antigen and purification of the antibodies can be performed subsequently, see, Harlow and Lane, supra, and the general descriptions of protein purification provided above.

Monoclonal antibodies are obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. The general principles and methods of recombinant polypeptide production discussed above are applicable for antibody production by recombinant methods.

When necessary, antibodies capable of specifically recognizing a mutant enzyme of the present invention can be tested for their cross-reactivity against the corresponding wild-type enzyme and thus distinguished from the antibodies against the wild-type enzyme. For instance, antisera obtained from an animal immunized with a mutant enzyme can be run through a column on which a corresponding wild-type enzyme is immobilized. The portion of the antisera that passes through the column recognizes only the mutant enzyme and not the corresponding wild-type enzyme. Similarly, monoclonal antibodies against a mutant enzyme can also be screened for their exclusivity in recognizing only the mutant but not the wild-type enzyme.

Polyclonal or monoclonal antibodies that specifically recognize only the mutant enzyme of the present invention but not the corresponding wild-type enzyme are useful for isolating the mutant enzyme from the wild-type enzyme, for example, by incubating a sample with a mutant enzyme-specific polyclonal or monoclonal antibody immobilized on a solid support.

### Immunoassays for Detecting Enzyme Expression

Once antibodies specific for an enzyme of the present invention are available, the amount of the polypeptide in a sample, e.g., a cell lysate, can be measured by a variety of immunoassay methods providing qualitative and quantitative results to a skilled artisan. For a review of immunological and immunoassay procedures in general see, e.g., Stites, supra; U.S. Pat. Nos. 4,366,241; 4,376,110; 4,517,288; and 4,837,168.

### Labeling in Immunoassays

Immunoassays often utilize a labeling agent to specifically bind to and label the binding complex formed by the antibody and the target protein. The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

### Immunoassay Formats

Immunoassays for detecting a target protein of interest (e.g., a recombinant enzyme) from samples may be either competitive or noncompetitive. Noncompetitive immunoassays are assays in which the amount of captured target protein is directly measured. In one preferred “sandwich” assay, for example, the antibody specific for the target protein can be bound directly to a solid substrate where the antibody is immobilized. It then captures the target protein in test samples. The antibody/target protein complex thus immobilized is then bound by a labeling agent, such as a second or third antibody bearing a label, as described above.

In competitive assays, the amount of target protein in a sample is measured indirectly by measuring the amount of an added (exogenous) target protein displaced (or competed away) from an antibody specific for the target protein by the target protein present in the sample. In a typical example of such an assay, the antibody is immobilized and the exogenous target protein is labeled. Since the amount of the exogenous target protein bound to the antibody is inversely proportional to the concentration of the target protein present in the sample, the target protein level in the sample can thus be determined based on the amount of exogenous target protein bound to the antibody and thus immobilized.

In some cases, western blot (immunoblot) analysis is used to detect and quantify the presence of a wild-type or mutant enzyme in the samples. The technique generally comprises separating sample proteins by gel electrophoresis on the basis of molecular weight, transferring the separated proteins to a suitable solid support (such as a nitrocellulose filter, a nylon filter, or a derivatized nylon filter) and incubating the samples with the antibodies that specifically bind the target protein. These antibodies may be directly labeled or alternatively may be subsequently detected using labeled antibodies (e.g., labeled sheep anti-mouse antibodies) that specifically bind to the antibodies against the enzyme.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

### Fusion Proteins

In other exemplary embodiments, the methods of the invention utilize fusion proteins that have more than one enzymatic activity that is involved in synthesis of a desired glycopeptide conjugate. The fusion polypeptides can be composed of, for example, a catalytically active domain of a glycosyltransferase that is joined to a catalytically active domain of an accessory enzyme. The accessory enzyme catalytic domain can, for example, catalyze a step in the formation of a nucleotide sugar that is a donor for the glycosyltransferase, or catalyze a reaction involved in a glycosyltransferase cycle. For example, a polynucleotide that encodes a glycosyltransferase can be joined, in-frame, to a polynucleotide that encodes an enzyme involved in nucleotide sugar synthesis. The resulting fusion protein can then catalyze not only the synthesis of the nucleotide sugar, but also the transfer of the sugar moiety to the acceptor molecule. The fusion protein can be two or more cycle enzymes linked into one expressible nucleotide sequence. In other embodiments the fusion protein includes the catalytically active domains of two or more glycosyltransferases. See, for example, 5,641,668. The modified glycopeptides of the present invention can be readily designed and manufactured utilizing various suitable fusion proteins (see, for example, PCT Patent Application PCT/CA98/01180, which was published as WO 99/31224 on Jun. 24, 1999.)

### Purification of Peptide- and Other-Conjugates

The products produced by the above processes can be used without purification. However, it is usually preferred to recover the product. Standard, well-known techniques for recovery of glycosylated saccharides such as thin or thick layer chromatography, column chromatography, ion exchange chromatography, or membrane filtration can be used. It is preferred to use membrane filtration, more preferably utilizing a reverse osmotic membrane, or one or more column chromatographic techniques for the recovery as is discussed hereinafter and in the literature cited herein. For instance, membrane filtration wherein the membranes have molecular weight cutoff of about 3000 to about 10,000 can be used to remove proteins such as glycosyl transferases. Nanofiltration or reverse osmosis can then be used to remove salts and/or purify the product saccharides (see, e.g., WO 98/15581). Nanofilter membranes are a class of reverse osmosis membranes that pass monovalent salts but retain polyvalent salts and uncharged solutes larger than about 100 to about 2,000 Daltons, depending upon the membrane used. Thus, in a typical application, saccharides prepared by the methods of the present invention will be retained in the membrane and contaminating salts will pass through.

If the modified glycoprotein is produced intracellularly, as a first step, the particulate debris, either host cells or lysed fragments, is removed, for example, by centrifugation or ultrafiltration; optionally, the protein may be concentrated with a commercially available protein concentration filter, followed by separating the polypeptide variant from other impurities by one or more steps selected from immunoaffinity chromatography, ion-exchange column fractionation (e.g., on diethylaminoethyl (DEAE) or matrices containing carboxymethyl or sulfopropyl groups), chromatography on Blue-Sepharose, CM Blue-Sepharose, MONO-Q, MONO-S, lentil lectin-Sepharose, WGA-Sepharose, Con A-Sepharose, Ether Toyopearl, Butyl Toyopearl, Phenyl Toyopearl, SP-Sepharose, or protein A Sepharose, SDS-PAGE chromatography, silica chromatography, chromatofocusing, reverse phase HPLC (e.g., silica gel with appended aliphatic groups), gel filtration using, e.g., Sephadex molecular sieve or size-exclusion chromatography, chromatography on columns that selectively bind the polypeptide, and ethanol or ammonium sulfate precipitation.

Modified glycopeptides produced in culture are usually isolated by initial extraction from cells, enzymes, etc., followed by one or more concentration, salting-out, aqueous ion-exchange, or size-exclusion chromatography steps, e.g., SP Sepharose. Additionally, the modified glycoprotein may be purified by affinity chromatography. HPLC may also be employed for one or more purification steps.

A protease inhibitor, e.g., methylsulfonylfluoride (PMSF) may be included in any of the foregoing steps to inhibit proteolysis and antibiotics may be included to prevent the growth of adventitious contaminants.

Within another embodiment, supernatants from systems which produce the modified glycopeptide of the invention are first concentrated using a commercially available protein concentration filter, for example, an Amicon or Millipore Pellicon ultrafiltration unit. Following the concentration step, the concentrate may be applied to a suitable purification matrix. For example, a suitable affinity matrix may comprise a ligand for the peptide, a lectin or antibody molecule bound to a suitable support. Alternatively, an anion-exchange resin may be employed, for example, a matrix or substrate having pendant DEAE groups. Suitable matrices include acrylamide, agarose, dextran, cellulose, or other types commonly employed in protein purification. Alternatively, a cation-exchange step may be employed. Suitable cation exchangers include various insoluble matrices comprising sulfopropyl or carboxymethyl groups. Sulfopropyl groups are particularly preferred.

Finally, one or more RP-HPLC steps employing hydrophobic RP-HPLC media, e.g., silica gel having pendant methyl or other aliphatic groups, may be employed to further purify a polypeptide variant composition. Some or all of the foregoing purification steps, in various combinations, can also be employed to provide a homogeneous modified glycoprotein.

The modified glycopeptide of the invention resulting from a large-scale fermentation may be purified by methods analogous to those disclosed by Urdal et al., J. Chromatog. 296: 171 (1984). This reference describes two sequential, RP-HPLC steps for purification of recombinant human IL-2 on a preparative HPLC column. Alternatively, techniques such as affinity chromatography may be utilized to purify the modified glycoprotein.

In another aspect, the invention provides a pharmaceutical composition. The pharmaceutical composition includes a pharmaceutically acceptable diluent and a covalent conjugate between a substrate (peptide, glycolipid, aglycone, etc.) and a modified sugar of the invention.

An exemplary conjugate is formed between a non-naturally-occurring, water-soluble polymer, therapeutic moiety or biomolecule and a glycosylated or non-glycosylated peptide. The polymer, therapeutic moiety or biomolecule is conjugated to the peptide via an intact glycosyl linking group interposed between and covalently linked to both the peptide and the polymer, therapeutic moiety or biomolecule.

Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable matrices, such as microspheres (e.g., polylactate polyglycolate), may also be employed as carriers for the pharmaceutical compositions of this invention. Suitable biodegradable microspheres are disclosed, for example, in U.S. Pat. Nos. 4,897,268 and 5,075,109.

Commonly, the pharmaceutical compositions are administered subcutaneously or parenterally, e.g., intravenously. Thus, the invention provides compositions for parenteral administration which comprise the compound dissolved or suspended in an acceptable carrier, preferably an aqueous carrier, e.g., water, buffered water, saline, PBS and the like. The compositions may also contain detergents such as Tween 20 and Tween 80; stabilizers such as mannitol, sorbitol, sucrose, and trehalose; and preservatives such as EDTA and m-cresol. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9 and most preferably from 7 and 8.

In some embodiments the glycopeptides of the invention can be incorporated into liposomes formed from standard vesicle-forming lipids. A variety of methods are available for preparing liposomes, as described in, e.g., Szoka et al., Ann. Rev. Biophys. Bioeng. 9: 467 (1980), U.S. Pat. Nos. 4,235,871, 4,501,728 and 4,837,028. The targeting of liposomes using a variety of targeting agents (e.g., the sialyl galactosides of the invention) is well known in the art (see, e.g., U.S. Pat. Nos. 4,957,773 and 4,603,044).

Standard methods for coupling targeting agents to liposomes can be used. These methods generally involve incorporation into liposomes of lipid components, such as phosphatidylethanolamine, which can be activated for attachment of targeting agents, or derivatized lipophilic compounds, such as lipid-derivatized glycopeptides of the invention.

Targeting mechanisms generally require that the targeting agents be positioned on the surface of the liposome in such a manner that the target moieties are available for interaction with the target, for example, a cell surface receptor. The carbohydrates of the invention may be attached to a lipid molecule before the liposome is formed using methods known to those of skill in the art (e.g., alkylation or acylation of a hydroxyl group present on the carbohydrate with a long chain alkyl halide or with a fatty acid, respectively). Alternatively, the liposome may be fashioned in such a way that a connector portion is first incorporated into the membrane at the time of forming the membrane. The connector portion must have a lipophilic portion, which is firmly embedded and anchored in the membrane. It must also have a reactive portion, which is chemically available on the aqueous surface of the liposome. The reactive portion is selected so that it will be chemically suitable to form a stable chemical bond with the targeting agent or carbohydrate, which is added later. In some cases it is possible to attach the target agent to the connector molecule directly, but in most instances it is more suitable to use a third molecule to act as a chemical bridge, thus linking the connector molecule which is in the membrane with the target agent or carbohydrate which is extended, three dimensionally, off of the vesicle surface.

The compounds prepared by the methods of the invention may also find use as diagnostic reagents. For example, labeled compounds can be used to locate areas of inflammation or tumor metastasis in a patient suspected of having an inflammation. For this use, the compounds can be labeled with ¹²⁵I, ¹⁴C, or tritium.

The following examples are provided to illustrate the conjugates, and methods and of the present invention, but not to limit the claimed invention.

While this invention has been disclosed with reference to specific embodiments, it is apparent that other embodiments and variations of this invention may be devised by others skilled in the art without departing from the true spirit and scope of the invention.

All patents, patent applications, and other publications cited in this application are incorporated by reference in the entirety.

1.-17. (canceled)

18. A method of preparing a peptide conjugate, which method comprises contacting a peptide comprising a glycosyl residue with:
(i) an acylating agent comprising an activated acyl moiety; and
(ii) an enzyme for which said acylating agent is a substrate under conditions appropriate to acylate said glycosyl residue thereby preparing said peptide conjugate, wherein said peptide conjugate comprises the glycosyl moiety: 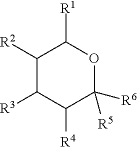
wherein
R¹, R², R³, R⁴, and R⁵ are members independently selected from H, OR^{7a}, N(R^{7a})₂, SR^{7a}, JC(O)R⁷, substituted or unsubstituted alkyl, substituted or unsubstituted heteroalkyl, substituted or unsubstituted aryl, substituted or unsubstituted heteroaryl, and substituted or unsubstituted heterocycloalkyl,
wherein
R⁷ is a member selected from H, OR⁸, NR⁸R⁹, substituted or unsubstituted alkyl, substituted or unsubstituted heteroalkyl, substituted or unsubstituted aryl, substituted or unsubstituted heteroaryl, and substituted or unsubstituted heterocycloalkyl,
wherein
R⁸ and R⁹ are members independently selected from H, substituted or unsubstituted alkyl, substituted or unsubstituted heteroalkyl, substituted or unsubstituted aryl, substituted or unsubstituted heteroaryl, and substituted or unsubstituted heterocycloalkyl,
R^{7a} is a member selected from H, substituted or unsubstituted alkyl, substituted or unsubstituted heteroalkyl, substituted or unsubstituted aryl, substituted or unsubstituted heteroaryl, and substituted or unsubstituted heterocycloalkyl,
J is a member selected from a bond, O, S and NH,
at least one of R¹, R², R³, R⁴, and R⁵ comprises a modifying group linked through an acyl group; and
R⁶ is a member selected from an amino acid residue of said peptide, a carbohydrate covalently bound to an amino acid residue of said peptide, and combinations thereof.

19. The method according to claim 18, wherein said glycosyl residue is acylated by said acylating agent at a site that is a member selected from an OH, NH₂, and SH.

20. The method according to claim 18, wherein said enzyme is a member selected from a lipase, a protease, an esterase, an acylase, and an acyltransferase.

21. The method according to claim 18, wherein said enzyme has an amino acid sequence that is a wild-type sequence for said enzyme.

22. The method according to claim 18, wherein said enzyme is a mutated enzyme wherein said mutated enzyme has a mutated amino acid sequence.

23. The method according to claim 22, wherein said mutated enzyme has an acylation activity that is enhanced relative to a corresponding wild-type enzyme.

24. The method according to claim 22, wherein said mutated amino acid sequence comprises a mutation wherein an amino acid residue implicated in hydrolysis of a member selected from an amide and an ester is replaced by an amino acid residue that is not implicated in said hydrolysis.

25. The method according to claim 18 wherein
R¹ is a member selected from: 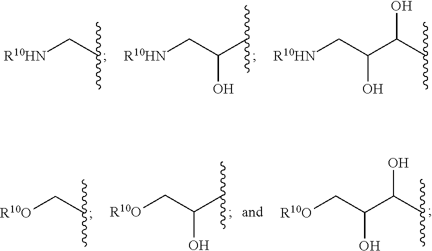
and R¹⁰ comprises a modifying group.

26. The method according to claim 25, wherein said glycosyl moiety is a sialic acid.

27. The method according to claim 18, wherein said modifying group is a member selected from poly(ether), poly(sialic acid), and poly(amino acid).

28. The method according to claim 25, wherein R¹⁰ has the formula: 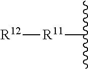
wherein
said linker is R¹¹, and R¹¹ is a member selected from substituted or unsubstituted alkyl and substituted or unsubstituted heteroalkyl;
said modifying group is R¹²; and [symbol]
represents a connection to the remainder of the conjugate through a member selected from O and N.

29. The method according to claim 28, wherein R¹¹ is an acyl moiety, such that said acyl moiety, taken together with the atoms to which it is covalently attached, comprises a moiety selected from an ester, amide, and urethane.

30. The method according to claim 18, wherein the glycosyl moiety has the structure: 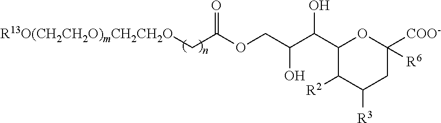
in which
m is an integer from 1 to 2500;
n is an integer from 0 to 40, and
R¹³ is a member selected from H and substituted or unsubstituted alkyl.

31. The method according to claim 18, wherein the glycosyl moiety has the structure: 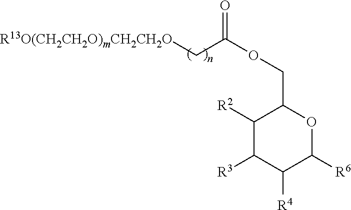
in which
m is an integer from 1 to 2500;
n is an integer from 0 to 40;
and R¹³ is a member selected from H and substituted or unsubstituted alkyl.

32. The method according to claim 31, wherein a member selected from R³ and R⁴ is N-acetyl.

33. The method according to claim 31, wherein R⁶ is a member selected from Gal, GalNAc, Man, GlcNAc and Glu.
